SEQUENCE LISTING (97 sequences)

SEQ ID NO: 1 - RNA, 135 nt, Artificial Sequence
  Synthetic Construct
ggguuuccgauccgccaucgugggugaguguaugcucuguggccgcgcucuggcuaguggcgcuacgcgucgcucucacgggugucgucggaucuaauccgucucuuuucgaaugcagguggagccgccgccacg

SEQ ID NO: 2 - RNA, 130 nt, Artificial Sequence
  Synthetic Construct
ggggauccgccaucgugggugaguguaugcucuguggccgcgcucuggcuaguggcgcuacgcgucgcucucacgggugucgucggaucuaauccgucucuuuucgaaugcagguggagccgccgccacg

SEQ ID NO: 3 - RNA, 130 nt, Artificial Sequence
  Synthetic Construct
ggggauccgccaucgugggugaguguuagcucuguggccgcgcucuggcuaguggcgcuacgcgucgcucucacgggugucgucggaucuaauccgucucuuuucgauagcagguggagccgccgccacg

SEQ ID NO: 4 - RNA, 47 nt, Artificial Sequence
  Synthetic Construct
gggaucuaauccgucucuuuucgauagcagguggagccgccgccacg

SEQ ID NO: 5 - RNA, 50 nt, Artificial Sequence
  Synthetic Construct
gggaucuaauccgucucuuuucgauagcagguggagccgccgccacgaug

SEQ ID NO: 6 - RNA, 289 nt, Mus musculus
gggaggaaagccucucuuaaucgcaucggcuguauaagaaagccuuuugaggcauuuuuuuuaguugagcacaucauuucgaggccauucugagguaaaccgagaaaagagcguaaagaaaccgagcgaacgagcaaaucuggcacugcguuagacagccgcgauuccgcugcagcgcgcaggcacguguguggccgccuaaggggcggguccuucggccaggagaccccgucggccacgcucggaucuuccuuuccgauccgccaucguggguggagccgccgccacg

SEQ ID NO: 7 - RNA, 281 nt, Artificial Sequence
  Synthetic Construct
gggaggaaagaaucgcaucggcuguauaagaaagccuuuugaggcauuuuuuuuaguugagcacaucauuucgaggccauucugagguaaaccgagaaaagagcguaaagaaaccgagcgaacgagcaaaucuggcacugcguuagacagccgcgauuccgcugcagcgcgcaggcacguguguggccgccuaaggggcggguccuucggccaggagaccccgucggccacgcucggaucuuccuuuccgauccgccaucguggguggagccgccgccacg

SEQ ID NO: 8 - RNA, 327 nt, Homo sapiens
gggccccucgaccuccuuuuaaaaauucucuuagccacguugauuguacgggaaaagccuuuuuaaaacaucuuuuacguugcuuaaaccuacaguuucgaaagcauuccgaaggcuaaagugagaaauaagcccaggcuagggagaggagaaacgaaguucacguccuagucuggcaccggguuggauugucgcugggacggcagucaggcauuugguguggucgccuaagggguggguccuucggcgggagcuccgggaaaccccgugggccugcgcggcguucuuccuuuucgauccgccaucugcgguggagccgccaccaaa

SEQ ID NO: 9 - RNA, 298 nt, Artificial Sequence
  Synthetic Construct
gggagccacguugauuguacgggaaaagccuuuuuaaaacaucuuuuacguugcuuaaaccuacaguuucgaaagcauuccgaaggcuaaagugagaaauaagcccaggcuagggagaggagaaacgaaguucacguccuagucuggcaccggguuggauugucgcugggacggcagucaggcauuugguguggucgccuaagggguggguccuucggcgggagcuccgggaaaccccgugggccugcgcggcguucuuccuuuucgauccgccaucugcgguggagccgccaccaaa

SEQ ID NO: 10 - RNA, 148 nt, Homo sapiens
gggcguucuuccuuuucgauccgccaucugcggugggugucugcacuucggcugcucucggguuagcacccuauggugccuucucuugugaucccugaccuaaccugucucuuccuuuuccucaaccucagguggagccgccaccaaa

SEQ ID NO: 11 - RNA, 137 nt, Artificial Sequence
  Synthetic Construct
gggcgcgauccgccaucugcggugggugucugcacuucggcugcucucggguuagcacccuauggugccuucucuugugaucccugaccuaaccugucucuuccuuuuccucaaccucagguggagccgccaccaaa

SEQ ID NO: 12 - RNA, 10 nt, Artificial Sequence
  Synthetic Construct
gggagccacc

SEQ ID NO: 13 - RNA, 20 nt, Artificial Sequence
  Synthetic Construct
ggggacagaaaacagccacc

SEQ ID NO: 14 - RNA, 30 nt, Artificial Sequence
  Synthetic Construct
gggaaagaaacaggacagaaaacagccacc

SEQ ID NO: 15 - RNA, 40 nt, Artificial Sequence
  Synthetic Construct
gggaacacauacaaaagaaacaggacagaaaacagccacc

SEQ ID NO: 16 - RNA, 50 nt, Artificial Sequence
  Synthetic Construct
gggaacgacaagaaacacauacaaaagaaacaggacagaaaacagccacc

SEQ ID NO: 17 - RNA, 60 nt, Artificial Sequence
  Synthetic Construct
gggcauaaacauaaacgacaagaaacacauacaaaagaaacaggacagaaaacagccacc

SEQ ID NO: 18 - RNA, 70 nt, Artificial Sequence
  Synthetic Construct
gggaagagauaaacauaaacauaaacgacaagaaacacauacaaaagaaacaggacagaaaacagccacc

SEQ ID NO: 19 - RNA, 100 nt, Artificial Sequence
  Synthetic Construct
gggaacaacagaggagaagagggaacaggacacaagagauaaacauaaacauaaacgacaagaaacacauacaaaagaaacaggacagaaaacagccacc

SEQ ID NO: 20 - RNA, 50 nt, Artificial Sequence
  Synthetic Construct
gggaaagaaaaagauaaggagaaaaauaaagagaggaagaaaaagccacc

SEQ ID NO: 21 - RNA, 50 nt, Artificial Sequence
  Synthetic Construct
gggaaaaguagaaagaaagaaagaagagaaaauaaagacaaagagccacc

SEQ ID NO: 22 - RNA, 70 nt, Artificial Sequence
  Synthetic Construct
gcuuucacuauuucauucauuucauucacacauuacacuuacaucacauccacauuacauuucugccacc

SEQ ID NO: 23 - RNA, 70 nt, Artificial Sequence
  Synthetic Construct
gcuuucacuauuucauucauuucauucucucauuacucuuacuucucuuccucauuacauuucugccacc

SEQ ID NO: 24 - RNA, 34 nt, Mus musculus
uuguguaugcguuaauaaaaagaaggaacucgua

SEQ ID NO: 25 - RNA, 178 nt, Homo sapiens
cuguaugaguuaauaaaagacaugaacuaacauuuauuguuggguuuuauugcaguaaaaagaaugguuuuuaagcaccaaauugauggucacaccauuuccuuuuaguagugcuacugcuaucgcugugugaauguugccucuggggauuaugugacccagugguucuguauaccug

SEQ ID NO: 26 - RNA, 276 nt, Homo sapiens
cuguaugaguuaauaaaagacaugaacuaacauuuauuguuggguuuuauugcaguaaaaagaaugguuuuuaagcaccaaauugauggucacaccauuuccuuuuaguagugcuacugcuaucgcugugugaauguugccucuggggauuaugugacccagugguucuguauaccugccaggugccaaccacuuguaaaggucuugauauuuucaauucuuagacuaccuauacuuuggcagaaguuauauuuaauguaaguugucuaaauauaa

SEQ ID NO: 27 - RNA, 2666 nt, Artificial Sequence
  Synthetic Construct
gauccgccaucgugggugaguguuagcucuguggccgcgcucuggcuaguggcgcuacgcgucgcucucacgggugucgucggaucuaauccgucucuuuucgauagcagguggagccgccgccacgauggaagggaagugguugcuguguauguuacuggugcuuggaacugcuauuguugaggcucaugauggacaugaugaugaugugauugauauugaggaugaccuugacgaugucauugaagagguagaagacucaaaaccagauaccacugcuccuccuucaucucccaagguuacuuacaaagcuccaguuccaacaggggaaguauauuuugcugauucuuuugacagaggaacucugucaggguggauuuuauccaaagccaagaaagacgauaccgaugaugaaauugccaaauaugauggaaagugggagguagaggaaaugaaggagucaaagcuuccaggugauaaaggacuuguguugaugucucgggccaagcaucaugccaucucugcuaaacugaacaagcccuuccuguuugacaccaagccucucauuguucaguaugagguuaauuuccaaaauggaauagaaugugguggugccuaugugaaacugcuuucuaaaacaccagaacucaaccuggaucaguuccaugacaagaccccuuauacgauuauguuugguccagauaaauguggagaggacuauaaacugcacuucaucuuccgacacaaaaaccccaaaacggguaucuaugaagaaaaacaugcuaagaggccagaugcagaucugaagaccuauuuuacugauaagaaaacacaucuuuacacacuaaucuugaauccagauaauaguuuugaaauacugguugaccaaucuguggugaauaguggaaaucugcucaaugacaugacuccuccuguaaauccuucacgugaaauugaggacccagaagaccggaagcccgaggauugggaugaaagaccaaaaaucccagauccagaagcugucaagccagaugacugggaugaagaugccccugcuaagauuccagaugaagaggccacaaaacccgaaggcugguuagaugaugagccugaguacguaccugauccagacgcagagaaaccugaggauugggaugaagacauggauggagaaugggaggcuccucagauugccaacccuagaugugagucagcuccuggauguggugucuggcagcgaccugugauugacaaccccaauuauaaaggcaaauggaagccuccuaugauugacaaucccaguuaccagggaaucuggaaacccaggaaaauaccaaauccagauuucuuugaagaucuggaaccuuucagaaugacuccuuuuagugcuauugguuuggagcugugguccaugaccucugacauuuuuuuugacaacuuuaucauuugugcugaucgaagaauaguugaugauugggccaaugauggauggggccugaagaaagcugcugauggggcugcugagccaggcguuguggggcagaugaacgaggcagcugaagagcgcccguggcuguggguagucuauauucuaacuguagcccuuccuguguuccugguuauccucuucugcuguucuggaaagaaacagaccagugguauggaguauaagaaaacugaugcaccucaaccggaugugaaggaagaggaagaagagaaggaagaggaaaaggacaagggagaugaggaggaggaaggagaagagaaacuugaagagaaacagaaaagugaugcugaagaagaugguggcacugucagucaagaggaggaagacagaaaaccuaaagcagaggaggaugaaauuuugaacagaucaccaagaaacagaaagccacgaagagagcucgaggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaagucuagaugauuguguaugcguuaauaaaaagaaggaacucgua

SEQ ID NO: 28 - RNA, 2657 nt, Artificial Sequence
  Synthetic Construct
gauccgccaucgugggugaguguuagcucuguggccgcgcucuggcuaguggcgcuacgcgucgcucucacgggugucgucggaucuaauccgucucuuuucgauagcagguggagccgccgccacgauggaagggaagugguugcuguguauguuacuggugcuuggaacugcuauuguugaggcucaugauggacaugaugaugaugugauugauauugaggaugaccuugacgaugucauugaagagguagaagacucaaaaccagauaccacugcuccuccuucaucucccaagguuacuuacaaagcuccaguuccaacaggggaaguauauuuugcugauucuuuugacagaggaacucugucaggguggauuuuauccaaagccaagaaagacgauaccgaugaugaaauugccaaauaugauggaaagugggagguagaggaaaugaaggagucaaagcuuccaggugauaaaggacuuguguugaugucucgggccaagcaucaugccaucucugcuaaacugaacaagcccuuccuguuugacaccaagccucucauuguucaguaugagguuaauuuccaaaauggaauagaaugugguggugccuaugugaaacugcuuucuaaaacaccagaacucaaccuggaucaguuccaugacaagaccccuuauacgauuauguuugguccagauaaauguggagaggacuauaaacugcacuucaucuuccgacacaaaaaccccaaaacggguaucuaugaagaaaaacaugcuaagaggccagaugcagaucugaagaccuauuuuacugauaagaaaacacaucuuuacacacuaaucuugaauccagauaauaguuuugaaauacugguugaccaaucuguggugaauaguggaaaucugcucaaugacaugacuccuccuguaaauccuucacgugaaauugaggacccagaagaccggaagcccgaggauugggaugaaagaccaaaaaucccagauccagaagcugucaagccagaugacugggaugaagaugccccugcuaagauuccagaugaagaggccacaaaacccgaaggcugguuagaugaugagccugaguacguaccugauccagacgcagagaaaccugaggauugggaugaagacauggauggagaaugggaggcuccucagauugccaacccuagaugugagucagcuccuggauguggugucuggcagcgaccugugauugacaaccccaauuauaaaggcaaauggaagccuccuaugauugacaaucccaguuaccagggaaucuggaaacccaggaaaauaccaaauccagauuucuuugaagaucuggaaccuuucagaaugacuccuuuuagugcuauugguuuggagcugugguccaugaccucugacauuuuuuuugacaacuuuaucauuugugcugaucgaagaauaguugaugauugggccaaugauggauggggccugaagaaagcugcugauggggcugcugagccaggcguuguggggcagaugaacgaggcagcugaagagcgcccguggcuguggguagucuauauucuaacuguagcccuuccuguguuccugguuauccucuucugcuguucuggaaagaaacagaccagugguauggaguauaagaaaacugaugcaccucaaccggaugugaaggaagaggaagaagagaaggaagaggaaaaggacaagggagaugaggaggaggaaggagaagagaaacuugaagagaaacagaaaagugaugcugaagaagaugguggcacugucagucaagaggaggaagacagaaaaccuaaagcagaggaggaugaaauuuugaacagaucaccaagaaacagaaagccacgaagagagcucgaggugagcaagggcgaggaggauaacauggccaucaucaaggaguucaugcgcuucaaggugcacauggagggcuccgugaacggccacgaguucgagaucgagggcgagggcgagggccgccccuacgagggcacccagaccgccaagcugaaggugaccaaggguggcccccugcccuucgccugggacauccuguccccucaguucauguacggcuccaaggccuacgugaagcaccccgccgacauccccgacuacuugaagcuguccuuccccgagggcuucaagugggagcgcgugaugaacuucgaggacggcggcguggugaccgugacccaggacuccucccugcaggacggcgaguucaucuacaaggugaagcugcgcggcaccaacuuccccuccgacggccccguaaugcagaagaagaccaugggcugggaggccuccuccgagcggauguaccccgaggacggcgcccugaagggcgagaucaagcagaggcugaagcugaaggacggcggccacuacgacgcugaggucaagaccaccuacaaggccaagaagcccgugcagcugcccggcgccuacaacgucaacaucaaguuggacaucaccucccacaacgaggacuacaccaucguggaacaguacgaacgcgccgagggccgccacuccaccggcggcauggacgagcuguacaagucuagaugauuguguaugcguuaauaaaaagaaggaacucgua

SEQ ID NO: 29 - RNA, 989 nt, Artificial Sequence
  Synthetic Construct
gauccgccaucgugggugaguguuagcucuguggccgcgcucuggcuaguggcgcuacgcgucgcucucacgggugucgucggaucuaauccgucucuuuucgauagcagguggagccgccgccacgauggugggacggaacagcgccaucgcugcaggagugugcggugcccucuucauaggguacugcaucuacuuugaccgcaaaaggaggagugaccccaaccucgaggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaagucuagaugauuguguaugcguuaauaaaaagaaggaacucgua

SEQ ID NO: 30 - RNA, 980 nt, Artificial Sequence
  Synthetic Construct
gauccgccaucgugggugaguguuagcucuguggccgcgcucuggcuaguggcgcuacgcgucgcucucacgggugucgucggaucuaauccgucucuuuucgauagcagguggagccgccgccacgauggugggacggaacagcgccaucgcugcaggagugugcggugcccucuucauaggguacugcaucuacuuugaccgcaaaaggaggagugaccccaaccucgaggugagcaagggcgaggaggauaacauggccaucaucaaggaguucaugcgcuucaaggugcacauggagggcuccgugaacggccacgaguucgagaucgagggcgagggcgagggccgccccuacgagggcacccagaccgccaagcugaaggugaccaaggguggcccccugcccuucgccugggacauccuguccccucaguucauguacggcuccaaggccuacgugaagcaccccgccgacauccccgacuacuugaagcuguccuuccccgagggcuucaagugggagcgcgugaugaacuucgaggacggcggcguggugaccgugacccaggacuccucccugcaggacggcgaguucaucuacaaggugaagcugcgcggcaccaacuuccccuccgacggccccguaaugcagaagaagaccaugggcugggaggccuccuccgagcggauguaccccgaggacggcgcccugaagggcgagaucaagcagaggcugaagcugaaggacggcggccacuacgacgcugaggucaagaccaccuacaaggccaagaagcccgugcagcugcccggcgccuacaacgucaacaucaaguuggacaucaccucccacaacgaggacuacaccaucguggaacaguacgaacgcgccgagggccgccacuccaccggcggcauggacgagcuguacaagucuagaugauuguguaugcguuaauaaaaagaaggaacucgua

SEQ ID NO: 31 - RNA, 1895 nt, Artificial Sequence
  Synthetic Construct
gauccgccaucgugggugaguguuagcucuguggccgcgcucuggcuaguggcgcuacgcgucgcucucacgggugucgucggaucuaauccgucucuuuucgauagcagguggagccgccgccacgauguggugguccuugauccuucuuucuugccugcuggcacugaccagugcccaugacaagccuuccuuccacccgcugucggaugaccugauuaacuauaucaacaaacagaauacaacauggcaggcuggacgcaacuucuacaauguugacauaagcuaucugaagaagcuguguggcacuguccuggguggacccaaacugccaggaaggguugcguucggugaggacauagaucuaccugaaaccuuugaugcacgggaacaaugguccaacugcccgaccauuggacagauuagagaccagggcuccugcggcucuuguugggcauuuggggcaguggaagccauuucugaccgaaccugcauucacaccaauggccgagucaacguggaggugucugcugaagaccugcuuacuugcugugguauccaguguggggacggcuguaaugguggcuaucccucuggagcauggagcuucuggacaaaaaaaggccugguuucagguggagucuacaauucucauguaggcugcuuaccauacaccaucccucccugcgagcaccaugucaauggcucccgucccccaugcacuggagaaggagauacucccaggugcaacaagagcugugaagcuggcuacuccccauccuacaaagaggauaagcacuuuggguacacuuccuacagcgugucuaacagugugaaggagaucauggcagaaaucuacaaaaauggcccaguggagggugccuucacuguguuuucugacuucuugacuuacaaaucaggaguauacaagcaugaagccggugauaugauggguggccacgccauccgcauccugggcuggggaguagagaauggaguucccuacuggcuggcagccaacucuuggaaccuugacuggggugauaauggcuucuuuaaaauccucagaggagaaaaccacuguggcauugaaucagaaauuguggcuggaaucccacgcacugaccaguacuggggaagauucgugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaagugauuguguaugcguuaauaaaaagaaggaacucgua

SEQ ID NO: 32 - RNA, 1886 nt, Artificial Sequence
  Synthetic Construct
gauccgccaucgugggugaguguuagcucuguggccgcgcucuggcuaguggcgcuacgcgucgcucucacgggugucgucggaucuaauccgucucuuuucgauagcagguggagccgccgccacgauguggugguccuugauccuucuuucuugccugcuggcacugaccagugcccaugacaagccuuccuuccacccgcugucggaugaccugauuaacuauaucaacaaacagaauacaacauggcaggcuggacgcaacuucuacaauguugacauaagcuaucugaagaagcuguguggcacuguccuggguggacccaaacugccaggaaggguugcguucggugaggacauagaucuaccugaaaccuuugaugcacgggaacaaugguccaacugcccgaccauuggacagauuagagaccagggcuccugcggcucuuguugggcauuuggggcaguggaagccauuucugaccgaaccugcauucacaccaauggccgagucaacguggaggugucugcugaagaccugcuuacuugcugugguauccaguguggggacggcuguaaugguggcuaucccucuggagcauggagcuucuggacaaaaaaaggccugguuucagguggagucuacaauucucauguaggcugcuuaccauacaccaucccucccugcgagcaccaugucaauggcucccgucccccaugcacuggagaaggagauacucccaggugcaacaagagcugugaagcuggcuacuccccauccuacaaagaggauaagcacuuuggguacacuuccuacagcgugucuaacagugugaaggagaucauggcagaaaucuacaaaaauggcccaguggagggugccuucacuguguuuucugacuucuugacuuacaaaucaggaguauacaagcaugaagccggugauaugauggguggccacgccauccgcauccugggcuggggaguagagaauggaguucccuacuggcuggcagccaacucuuggaaccuugacuggggugauaauggcuucuuuaaaauccucagaggagaaaaccacuguggcauugaaucagaaauuguggcuggaaucccacgcacugaccaguacuggggaagauucgugagcaagggcgaggaggauaacauggccaucaucaaggaguucaugcgcuucaaggugcacauggagggcuccgugaacggccacgaguucgagaucgagggcgagggcgagggccgccccuacgagggcacccagaccgccaagcugaaggugaccaaggguggcccccugcccuucgccugggacauccuguccccucaguucauguacggcuccaaggccuacgugaagcaccccgccgacauccccgacuacuugaagcuguccuuccccgagggcuucaagugggagcgcgugaugaacuucgaggacggcggcguggugaccgugacccaggacuccucccugcaggacggcgaguucaucuacaaggugaagcugcgcggcaccaacuuccccuccgacggccccguaaugcagaagaagaccaugggcugggaggccuccuccgagcggauguaccccgaggacggcgcccugaagggcgagaucaagcagaggcugaagcugaaggacggcggccacuacgacgcugaggucaagaccaccuacaaggccaagaagcccgugcagcugcccggcgccuacaacgucaacaucaaguuggacaucaccucccacaacgaggacuacaccaucguggaacaguacgaacgcgccgagggccgccacuccaccggcggcauggacgagcuguacaagugauuguguaugcguuaauaaaaagaaggaacucgua

SEQ ID NO: 33 - RNA, 985 nt, Artificial Sequence
  Synthetic Construct
gauccgccaucgugggugaguguuagcucuguggccgcgcucuggcuaguggcgcuacgcgucgcucucacgggugucgucggaucuaauccgucucuuuucgauagcagguggagccgccgccacgauggccccaaagaagaagcggaaggucgguauccacggagucccagcagccgugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaagaagcguccugcugcuacuaagaaagcuggucaagcuaagaaaaagaaauaagcggccgcuuguguaugcguuaauaaaaagaaggaacucgua

SEQ ID NO: 34 - RNA, 979 nt, Artificial Sequence
  Synthetic Construct
ggggauccgccaucgugggugaguguuagcucuguggccgcgcucuggcuaguggcgcuacgcgucgcucucacgggugucgucggaucuaauccgucucuuuucgauagcagguggagccgccgccacgauggccccaaagaagaagcggaaggucgguauccacggagucccagcagccgugagcaagggcgaggaggauaacauggccaucaucaaggaguucaugcgcuucaaggugcacauggagggcuccgugaacggccacgaguucgagaucgagggcgagggcgagggccgccccuacgagggcacccagaccgccaagcugaaggugaccaaggguggcccccugcccuucgccugggacauccuguccccucaguucauguacggcuccaaggccuacgugaagcaccccgccgacauccccgacuacuugaagcuguccuuccccgagggcuucaagugggagcgcgugaugaacuucgaggacggcggcguggugaccgugacccaggacuccucccugcaggacggcgaguucaucuacaaggugaagcugcgcggcaccaacuuccccuccgacggccccguaaugcagaagaagaccaugggcugggaggccuccuccgagcggauguaccccgaggacggcgcccugaagggcgagaucaagcagaggcugaagcugaaggacggcggccacuacgacgcugaggucaagaccaccuacaaggccaagaagcccgugcagcugcccggcgccuacaacgucaacaucaaguuggacaucaccucccacaacgaggacuacaccaucguggaacaguacgaacgcgccgagggccgccacuccaccggcggcauggacgagcuguacaagaagcguccugcugcuacuaagaaagcuggucaagcuaagaaaaagaaauaagcggccgcuuguguaugcguuaauaaaaagaaggaacucgua

SEQ ID NO: 35 - RNA, 3530 nt, Artificial Sequence
  Synthetic Construct
gauccgccaucgugggugaguguuagcucuguggccgcgcucuggcuaguggcgcuacgcgucgcucucacgggugucgucggaucuaauccgucucuuuucgauagcagguggagccgccgccacgauggugggacggaacagcgccaucgcugcaggagugugcggugcccucuucauaggguacugcaucuacuuugaccgcaaaaggaggagugaccccaaccucgaggugagcaagggcgaggaggauaacauggccaucaucaaggaguucaugcgcuucaaggugcacauggagggcuccgugaacggccacgaguucgagaucgagggcgagggcgagggccgccccuacgagggcacccagaccgccaagcugaaggugaccaaggguggcccccugcccuucgccugggacauccuguccccucaguucauguacggcuccaaggccuacgugaagcaccccgccgacauccccgacuacuugaagcuguccuuccccgagggcuucaagugggagcgcgugaugaacuucgaggacggcggcguggugaccgugacccaggacuccucccugcaggacggcgaguucaucuacaaggugaagcugcgcggcaccaacuuccccuccgacggccccguaaugcagaagaagaccaugggcugggaggccuccuccgagcggauguaccccgaggacggcgcccugaagggcgagaucaagcagaggcugaagcugaaggacggcggccacuacgacgcugaggucaagaccaccuacaaggccaagaagcccgugcagcugcccggcgccuacaacgucaacaucaaguuggacaucaccucccacaacgaggacuacaccaucguggaacaguacgaacgcgccgagggccgccacuccaccggcggcauggacgagcuguacaagggauccggcgcaacaaacuucucucugcugaaacaagccggagaugucgaagagaauccuggaccgauggaagggaagugguugcuguguauguuacuggugcuuggaacugcuauuguugaggcucaugauggacaugaugaugaugugauugauauugaggaugaccuugacgaugucauugaagagguagaagacucaaaaccagauaccacugcuccuccuucaucucccaagguuacuuacaaagcuccaguuccaacaggggaaguauauuuugcugauucuuuugacagaggaacucugucaggguggauuuuauccaaagccaagaaagacgauaccgaugaugaaauugccaaauaugauggaaagugggagguagaggaaaugaaggagucaaagcuuccaggugauaaaggacuuguguugaugucucgggccaagcaucaugccaucucugcuaaacugaacaagcccuuccuguuugacaccaagccucucauuguucaguaugagguuaauuuccaaaauggaauagaaugugguggugccuaugugaaacugcuuucuaaaacaccagaacucaaccuggaucaguuccaugacaagaccccuuauacgauuauguuugguccagauaaauguggagaggacuauaaacugcacuucaucuuccgacacaaaaaccccaaaacggguaucuaugaagaaaaacaugcuaagaggccagaugcagaucugaagaccuauuuuacugauaagaaaacacaucuuuacacacuaaucuugaauccagauaauaguuuugaaauacugguugaccaaucuguggugaauaguggaaaucugcucaaugacaugacuccuccuguaaauccuucacgugaaauugaggacccagaagaccggaagcccgaggauugggaugaaagaccaaaaaucccagauccagaagcugucaagccagaugacugggaugaagaugccccugcuaagauuccagaugaagaggccacaaaacccgaaggcugguuagaugaugagccugaguacguaccugauccagacgcagagaaaccugaggauugggaugaagacauggauggagaaugggaggcuccucagauugccaacccuagaugugagucagcuccuggauguggugucuggcagcgaccugugauugacaaccccaauuauaaaggcaaauggaagccuccuaugauugacaaucccaguuaccagggaaucuggaaacccaggaaaauaccaaauccagauuucuuugaagaucuggaaccuuucagaaugacuccuuuuagugcuauugguuuggagcugugguccaugaccucugacauuuuuuuugacaacuuuaucauuugugcugaucgaagaauaguugaugauugggccaaugauggauggggccugaagaaagcugcugauggggcugcugagccaggcguuguggggcagaugaacgaggcagcugaagagcgcccguggcuguggguagucuauauucuaacuguagcccuuccuguguuccugguuauccucuucugcuguucuggaaagaaacagaccagugguauggaguauaagaaaacugaugcaccucaaccggaugugaaggaagaggaagaagagaaggaagaggaaaaggacaagggagaugaggaggaggaaggagaagagaaacuugaagagaaacagaaaagugaugcugaagaagaugguggcacugucagucaagaggaggaagacagaaaaccuaaagcagaggaggaugaaauuuugaacagaucaccaagaaacagaaagccacgaagagaggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaagugauuguguaugcguuaauaaaaagaaggaacucgua

SEQ ID NO: 36 - RNA, 2771 nt, Artificial Sequence
  Synthetic Construct
gauccgccaucgugggugaguguuagcucuguggccgcgcucuggcuaguggcgcuacgcgucgcucucacgggugucgucggaucuaauccgucucuuuucgauagcagguggagccgccgccacgauggugggacggaacagcgccaucgcugcaggagugugcggugcccucuucauaggguacugcaucuacuuugaccgcaaaaggaggagugaccccaaccucgaggugagcaagggcgaggaggauaacauggccaucaucaaggaguucaugcgcuucaaggugcacauggagggcuccgugaacggccacgaguucgagaucgagggcgagggcgagggccgccccuacgagggcacccagaccgccaagcugaaggugaccaaggguggcccccugcccuucgccugggacauccuguccccucaguucauguacggcuccaaggccuacgugaagcaccccgccgacauccccgacuacuugaagcuguccuuccccgagggcuucaagugggagcgcgugaugaacuucgaggacggcggcguggugaccgugacccaggacuccucccugcaggacggcgaguucaucuacaaggugaagcugcgcggcaccaacuuccccuccgacggccccguaaugcagaagaagaccaugggcugggaggccuccuccgagcggauguaccccgaggacggcgcccugaagggcgagaucaagcagaggcugaagcugaaggacggcggccacuacgacgcugaggucaagaccaccuacaaggccaagaagcccgugcagcugcccggcgccuacaacgucaacaucaaguuggacaucaccucccacaacgaggacuacaccaucguggaacaguacgaacgcgccgagggccgccacuccaccggcggcauggacgagcuguacaagggauccggcgcaacaaacuucucucugcugaaacaagccggagaugucgaagagaauccuggaccgauguggugguccuugauccuucuuucuugccugcuggcacugaccagugcccaugacaagccuuccuuccacccgcugucggaugaccugauuaacuauaucaacaaacagaauacaacauggcaggcuggacgcaacuucuacaauguugacauaagcuaucugaagaagcuguguggcacuguccuggguggacccaaacugccaggaaggguugcguucggugaggacauagaucuaccugaaaccuuugaugcacgggaacaaugguccaacugcccgaccauuggacagauuagagaccagggcuccugcggcucuuguugggcauuuggggcaguggaagccauuucugaccgaaccugcauucacaccaauggccgagucaacguggaggugucugcugaagaccugcuuacuugcugugguauccaguguggggacggcuguaaugguggcuaucccucuggagcauggagcuucuggacaaaaaaaggccugguuucagguggagucuacaauucucauguaggcugcuuaccauacaccaucccucccugcgagcaccaugucaauggcucccgucccccaugcacuggagaaggagauacucccaggugcaacaagagcugugaagcuggcuacuccccauccuacaaagaggauaagcacuuuggguacacuuccuacagcgugucuaacagugugaaggagaucauggcagaaaucuacaaaaauggcccaguggagggugccuucacuguguuuucugacuucuugacuuacaaaucaggaguauacaagcaugaagccggugauaugauggguggccacgccauccgcauccugggcuggggaguagagaauggaguucccuacuggcuggcagccaacucuuggaaccuugacuggggugauaauggcuucuuuaaaauccucagaggagaaaaccacuguggcauugaaucagaaauuguggcuggaaucccacgcacugaccaguacuggggaagauucgugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaagugauuguguaugcguuaauaaaaagaaggaacucgua

SEQ ID NO: 37 - RNA, 1861 nt, Artificial Sequence
  Synthetic Construct
gauccgccaucgugggugaguguuagcucuguggccgcgcucuggcuaguggcgcuacgcgucgcucucacgggugucgucggaucuaauccgucucuuuucgauagcagguggagccgccgccacgauggugggacggaacagcgccaucgcugcaggagugugcggugcccucuucauaggguacugcaucuacuuugaccgcaaaaggaggagugaccccaaccucgaggugagcaagggcgaggaggauaacauggccaucaucaaggaguucaugcgcuucaaggugcacauggagggcuccgugaacggccacgaguucgagaucgagggcgagggcgagggccgccccuacgagggcacccagaccgccaagcugaaggugaccaaggguggcccccugcccuucgccugggacauccuguccccucaguucauguacggcuccaaggccuacgugaagcaccccgccgacauccccgacuacuugaagcuguccuuccccgagggcuucaagugggagcgcgugaugaacuucgaggacggcggcguggugaccgugacccaggacuccucccugcaggacggcgaguucaucuacaaggugaagcugcgcggcaccaacuuccccuccgacggccccguaaugcagaagaagaccaugggcugggaggccuccuccgagcggauguaccccgaggacggcgcccugaagggcgagaucaagcagaggcugaagcugaaggacggcggccacuacgacgcugaggucaagaccaccuacaaggccaagaagcccgugcagcugcccggcgccuacaacgucaacaucaaguuggacaucaccucccacaacgaggacuacaccaucguggaacaguacgaacgcgccgagggccgccacuccaccggcggcauggacgagcuguacaagggauccggcgcaacaaacuucucucugcugaaacaagccggagaugucgaagagaauccuggaccgauggccccaaagaagaagcggaaggucgguauccacggagucccagcagccgugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaagaagcguccugcugcuacuaagaaagcuggucaagcuaagaaaaagaaauaagcggccgcuuguguaugcguuaauaaaaagaaggaacucgua

SEQ ID NO: 38 - RNA, 3524 nt, Artificial Sequence
  Synthetic Construct
gauccgccaucgugggugaguguuagcucuguggccgcgcucuggcuaguggcgcuacgcgucgcucucacgggugucgucggaucuaauccgucucuuuucgauagcagguggagccgccgccacgauggugggacggaacagcgccaucgcugcaggagugugcggugcccucuucauaggguacugcaucuacuuugaccgcaaaaggaggagugaccccaaccucgaggugagcaagggcgaggaggauaacauggccaucaucaaggaguucaugcgcuucaaggugcacauggagggcuccgugaacggccacgaguucgagaucgagggcgagggcgagggccgccccuacgagggcacccagaccgccaagcugaaggugaccaaggguggcccccugcccuucgccugggacauccuguccccucaguucauguacggcuccaaggccuacgugaagcaccccgccgacauccccgacuacuugaagcuguccuuccccgagggcuucaagugggagcgcgugaugaacuucgaggacggcggcguggugaccgugacccaggacuccucccugcaggacggcgaguucaucuacaaggugaagcugcgcggcaccaacuuccccuccgacggccccguaaugcagaagaagaccaugggcugggaggccuccuccgagcggauguaccccgaggacggcgcccugaagggcgagaucaagcagaggcugaagcugaaggacggcggccacuacgacgcugaggucaagaccaccuacaaggccaagaagcccgugcagcugcccggcgccuacaacgucaacaucaaguuggacaucaccucccacaacgaggacuacaccaucguggaacaguacgaacgcgccgagggccgccacuccaccggcggcauggacgagcuguacaagggagguggaggcagcggaggcgggggcaguggaggaggggguuccggugguggugguaguauggaagggaagugguugcuguguauguuacuggugcuuggaacugcuauuguugaggcucaugauggacaugaugaugaugugauugauauugaggaugaccuugacgaugucauugaagagguagaagacucaaaaccagauaccacugcuccuccuucaucucccaagguuacuuacaaagcuccaguuccaacaggggaaguauauuuugcugauucuuuugacagaggaacucugucaggguggauuuuauccaaagccaagaaagacgauaccgaugaugaaauugccaaauaugauggaaagugggagguagaggaaaugaaggagucaaagcuuccaggugauaaaggacuuguguugaugucucgggccaagcaucaugccaucucugcuaaacugaacaagcccuuccuguuugacaccaagccucucauuguucaguaugagguuaauuuccaaaauggaauagaaugugguggugccuaugugaaacugcuuucuaaaacaccagaacucaaccuggaucaguuccaugacaagaccccuuauacgauuauguuugguccagauaaauguggagaggacuauaaacugcacuucaucuuccgacacaaaaaccccaaaacggguaucuaugaagaaaaacaugcuaagaggccagaugcagaucugaagaccuauuuuacugauaagaaaacacaucuuuacacacuaaucuugaauccagauaauaguuuugaaauacugguugaccaaucuguggugaauaguggaaaucugcucaaugacaugacuccuccuguaaauccuucacgugaaauugaggacccagaagaccggaagcccgaggauugggaugaaagaccaaaaaucccagauccagaagcugucaagccagaugacugggaugaagaugccccugcuaagauuccagaugaagaggccacaaaacccgaaggcugguuagaugaugagccugaguacguaccugauccagacgcagagaaaccugaggauugggaugaagacauggauggagaaugggaggcuccucagauugccaacccuagaugugagucagcuccuggauguggugucuggcagcgaccugugauugacaaccccaauuauaaaggcaaauggaagccuccuaugauugacaaucccaguuaccagggaaucuggaaacccaggaaaauaccaaauccagauuucuuugaagaucuggaaccuuucagaaugacuccuuuuagugcuauugguuuggagcugugguccaugaccucugacauuuuuuuugacaacuuuaucauuugugcugaucgaagaauaguugaugauugggccaaugauggauggggccugaagaaagcugcugauggggcugcugagccaggcguuguggggcagaugaacgaggcagcugaagagcgcccguggcuguggguagucuauauucuaacuguagcccuuccuguguuccugguuauccucuucugcuguucuggaaagaaacagaccagugguauggaguauaagaaaacugaugcaccucaaccggaugugaaggaagaggaagaagagaaggaagaggaaaaggacaagggagaugaggaggaggaaggagaagagaaacuugaagagaaacagaaaagugaugcugaagaagaugguggcacugucagucaagaggaggaagacagaaaaccuaaagcagaggaggaugaaauuuugaacagaucaccaagaaacagaaagccacgaagagaggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaagugauuguguaugcguuaauaaaaagaaggaacucgua

SEQ ID NO: 39 - RNA, 2765 nt, Artificial Sequence
  Synthetic Construct
gauccgccaucgugggugaguguuagcucuguggccgcgcucuggcuaguggcgcuacgcgucgcucucacgggugucgucggaucuaauccgucucuuuucgauagcagguggagccgccgccacgauggugggacggaacagcgccaucgcugcaggagugugcggugcccucuucauaggguacugcaucuacuuugaccgcaaaaggaggagugaccccaaccucgaggugagcaagggcgaggaggauaacauggccaucaucaaggaguucaugcgcuucaaggugcacauggagggcuccgugaacggccacgaguucgagaucgagggcgagggcgagggccgccccuacgagggcacccagaccgccaagcugaaggugaccaaggguggcccccugcccuucgccugggacauccuguccccucaguucauguacggcuccaaggccuacgugaagcaccccgccgacauccccgacuacuugaagcuguccuuccccgagggcuucaagugggagcgcgugaugaacuucgaggacggcggcguggugaccgugacccaggacuccucccugcaggacggcgaguucaucuacaaggugaagcugcgcggcaccaacuuccccuccgacggccccguaaugcagaagaagaccaugggcugggaggccuccuccgagcggauguaccccgaggacggcgcccugaagggcgagaucaagcagaggcugaagcugaaggacggcggccacuacgacgcugaggucaagaccaccuacaaggccaagaagcccgugcagcugcccggcgccuacaacgucaacaucaaguuggacaucaccucccacaacgaggacuacaccaucguggaacaguacgaacgcgccgagggccgccacuccaccggcggcauggacgagcuguacaagggagguggaggcagcggaggcgggggcaguggaggaggggguuccggugguggugguaguauguggugguccuugauccuucuuucuugccugcuggcacugaccagugcccaugacaagccuuccuuccacccgcugucggaugaccugauuaacuauaucaacaaacagaauacaacauggcaggcuggacgcaacuucuacaauguugacauaagcuaucugaagaagcuguguggcacuguccuggguggacccaaacugccaggaaggguugcguucggugaggacauagaucuaccugaaaccuuugaugcacgggaacaaugguccaacugcccgaccauuggacagauuagagaccagggcuccugcggcucuuguugggcauuuggggcaguggaagccauuucugaccgaaccugcauucacaccaauggccgagucaacguggaggugucugcugaagaccugcuuacuugcugugguauccaguguggggacggcuguaaugguggcuaucccucuggagcauggagcuucuggacaaaaaaaggccugguuucagguggagucuacaauucucauguaggcugcuuaccauacaccaucccucccugcgagcaccaugucaauggcucccgucccccaugcacuggagaaggagauacucccaggugcaacaagagcugugaagcuggcuacuccccauccuacaaagaggauaagcacuuuggguacacuuccuacagcgugucuaacagugugaaggagaucauggcagaaaucuacaaaaauggcccaguggagggugccuucacuguguuuucugacuucuugacuuacaaaucaggaguauacaagcaugaagccggugauaugauggguggccacgccauccgcauccugggcuggggaguagagaauggaguucccuacuggcuggcagccaacucuuggaaccuugacuggggugauaauggcuucuuuaaaauccucagaggagaaaaccacuguggcauugaaucagaaauuguggcuggaaucccacgcacugaccaguacuggggaagauucgugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaagugauuguguaugcguuaauaaaaagaaggaacucgua

SEQ ID NO: 40 - RNA, 1855 nt, Artificial Sequence
  Synthetic Construct
gauccgccaucgugggugaguguuagcucuguggccgcgcucuggcuaguggcgcuacgcgucgcucucacgggugucgucggaucuaauccgucucuuuucgauagcagguggagccgccgccacgauggugggacggaacagcgccaucgcugcaggagugugcggugcccucuucauaggguacugcaucuacuuugaccgcaaaaggaggagugaccccaaccucgaggugagcaagggcgaggaggauaacauggccaucaucaaggaguucaugcgcuucaaggugcacauggagggcuccgugaacggccacgaguucgagaucgagggcgagggcgagggccgccccuacgagggcacccagaccgccaagcugaaggugaccaaggguggcccccugcccuucgccugggacauccuguccccucaguucauguacggcuccaaggccuacgugaagcaccccgccgacauccccgacuacuugaagcuguccuuccccgagggcuucaagugggagcgcgugaugaacuucgaggacggcggcguggugaccgugacccaggacuccucccugcaggacggcgaguucaucuacaaggugaagcugcgcggcaccaacuuccccuccgacggccccguaaugcagaagaagaccaugggcugggaggccuccuccgagcggauguaccccgaggacggcgcccugaagggcgagaucaagcagaggcugaagcugaaggacggcggccacuacgacgcugaggucaagaccaccuacaaggccaagaagcccgugcagcugcccggcgccuacaacgucaacaucaaguuggacaucaccucccacaacgaggacuacaccaucguggaacaguacgaacgcgccgagggccgccacuccaccggcggcauggacgagcuguacaagggagguggaggcagcggaggcgggggcaguggaggaggggguuccggugguggugguaguauggccccaaagaagaagcggaaggucgguauccacggagucccagcagccgugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaagaagcguccugcugcuacuaagaaagcuggucaagcuaagaaaaagaaauaagcggccgcuuguguaugcguuaauaaaaagaaggaacucgua

SEQ ID NO: 41 - DNA, 135 bp, Mus musculus
gggtttccgatccgccatcgtgggtgagtgtatgctctgtggccgcgctctggctagtggcgctacgcgtcgctctcacgggtgtcgtcggatctaatccgtctcttttcgaatgcaggtggagccgccgccacg

SEQ ID NO: 42 - DNA, 130 bp, Artificial Sequence
  Synthetic Construct
ggggatccgccatcgtgggtgagtgtatgctctgtggccgcgctctggctagtggcgctacgcgtcgctctcacgggtgtcgtcggatctaatccgtctcttttcgaatgcaggtggagccgccgccacg

SEQ ID NO: 43 - DNA, 130 bp, Artificial Sequence
  Synthetic Construct
ggggatccgccatcgtgggtgagtgttagctctgtggccgcgctctggctagtggcgctacgcgtcgctctcacgggtgtcgtcggatctaatccgtctcttttcgatagcaggtggagccgccgccacg

SEQ ID NO: 44 - DNA, 47 bp, Artificial Sequence
  Synthetic Construct
gggatctaatccgtctcttttcgatagcaggtggagccgccgccacg

SEQ ID NO: 45 - DNA, 50 bp, Artificial Sequence
  Synthetic Construct
gggatctaatccgtctcttttcgatagcaggtggagccgccgccacgatg

SEQ ID NO: 46 - DNA, 289 bp, Mus musculus
gggaggaaagcctctcttaatcgcatcggctgtataagaaagccttttgaggcattttttttagttgagcacatcatttcgaggccattctgaggtaaaccgagaaaagagcgtaaagaaaccgagcgaacgagcaaatctggcactgcgttagacagccgcgattccgctgcagcgcgcaggcacgtgtgtggccgcctaaggggcgggtccttcggccaggagaccccgtcggccacgctcggatcttcctttccgatccgccatcgtgggtggagccgccgccacg

SEQ ID NO: 47 - DNA, 281 bp, Artificial Sequence
  Synthetic Construct
gggaggaaagaatcgcatcggctgtataagaaagccttttgaggcattttttttagttgagcacatcatttcgaggccattctgaggtaaaccgagaaaagagcgtaaagaaaccgagcgaacgagcaaatctggcactgcgttagacagccgcgattccgctgcagcgcgcaggcacgtgtgtggccgcctaaggggcgggtccttcggccaggagaccccgtcggccacgctcggatcttcctttccgatccgccatcgtgggtggagccgccgccacg

SEQ ID NO: 48 - DNA, 327 bp, Artificial Sequence
  Synthetic Construct
gggcccctcgacctccttttaaaaattctcttagccacgttgattgtacgggaaaagcctttttaaaacatcttttacgttgcttaaacctacagtttcgaaagcattccgaaggctaaagtgagaaataagcccaggctagggagaggagaaacgaagttcacgtcctagtctggcaccgggttggattgtcgctgggacggcagtcaggcatttggtgtggtcgcctaaggggtgggtccttcggcgggagctccgggaaaccccgtgggcctgcgcggcgttcttccttttcgatccgccatctgcggtggagccgccaccaaa

SEQ ID NO: 49 - DNA, 298 bp, Artificial Sequence
  Synthetic Construct
gggagccacgttgattgtacgggaaaagcctttttaaaacatcttttacgttgcttaaacctacagtttcgaaagcattccgaaggctaaagtgagaaataagcccaggctagggagaggagaaacgaagttcacgtcctagtctggcaccgggttggattgtcgctgggacggcagtcaggcatttggtgtggtcgcctaaggggtgggtccttcggcgggagctccgggaaaccccgtgggcctgcgcggcgttcttccttttcgatccgccatctgcggtggagccgccaccaaa

SEQ ID NO: 50 - DNA, 148 bp, Homo sapiens
gggcgttcttccttttcgatccgccatctgcggtgggtgtctgcacttcggctgctctcgggttagcaccctatggtgccttctcttgtgatccctgacctaacctgtctcttccttttcctcaacctcaggtggagccgccaccaaa

SEQ ID NO: 51 - DNA, 137 bp, Artificial Sequence
  Synthetic Construct
gggcgcgatccgccatctgcggtgggtgtctgcacttcggctgctctcgggttagcaccctatggtgccttctcttgtgatccctgacctaacctgtctcttccttttcctcaacctcaggtggagccgccaccaaa

SEQ ID NO: 52 - DNA, 10 bp, Artificial Sequence
  Synthetic Construct
gggagccacc

SEQ ID NO: 53 - DNA, 20 bp, Artificial Sequence
  Synthetic Construct
ggggacagaaaacagccacc

SEQ ID NO: 54 - DNA, 30 bp, Artificial Sequence
  Synthetic Construct
gggaaagaaacaggacagaaaacagccacc

SEQ ID NO: 55 - DNA, 40 bp, Artificial Sequence
  Synthetic Construct
gggaacacatacaaaagaaacaggacagaaaacagccacc

SEQ ID NO: 56 - DNA, 50 bp, Artificial Sequence
  Synthetic Construct
gggaacgacaagaaacacatacaaaagaaacaggacagaaaacagccacc

SEQ ID NO: 57 - DNA, 60 bp, Artificial Sequence
  Synthetic Construct
gggcataaacataaacgacaagaaacacatacaaaagaaacaggacagaaaacagccacc

SEQ ID NO: 58 - DNA, 70 bp, Artificial Sequence
  Synthetic Construct
gggaagagataaacataaacataaacgacaagaaacacatacaaaagaaacaggacagaaaacagccacc

SEQ ID NO: 59 - DNA, 100 bp, Artificial Sequence
  Synthetic Construct
gggaacaacagaggagaagagggaacaggacacaagagataaacataaacataaacgacaagaaacacatacaaaagaaacaggacagaaaacagccacc

SEQ ID NO: 60 - DNA, 50 bp, Artificial Sequence
  Synthetic Construct
gggaaagaaaaagataaggagaaaaataaagagaggaagaaaaagccacc

SEQ ID NO: 61 - DNA, 50 bp, Artificial Sequence
  Synthetic Construct
gggaaaagtagaaagaaagaaagaagagaaaataaagacaaagagccacc

SEQ ID NO: 62 - DNA, 70 bp, Artificial Sequence
  Synthetic Construct
gctttcactatttcattcatttcattcacacattacacttacatcacatccacattacatttctgccacc

SEQ ID NO: 63 - DNA, 70 bp, Artificial Sequence
  Synthetic Construct
gctttcactatttcattcatttcattctctcattactcttacttctcttcctcattacatttctgccacc

SEQ ID NO: 64 - DNA, 34 bp, Mus musculus
ttgtgtatgcgttaataaaaagaaggaactcgta

SEQ ID NO: 65 - DNA, 178 bp, Homo sapiens
ctgtatgagttaataaaagacatgaactaacatttattgttgggttttattgcagtaaaaagaatggtttttaagcaccaaattgatggtcacaccatttccttttagtagtgctactgctatcgctgtgtgaatgttgcctctggggattatgtgacccagtggttctgtatacctg

SEQ ID NO: 66 - DNA, 276 bp, Homo sapiens
ctgtatgagttaataaaagacatgaactaacatttattgttgggttttattgcagtaaaaagaatggtttttaagcaccaaattgatggtcacaccatttccttttagtagtgctactgctatcgctgtgtgaatgttgcctctggggattatgtgacccagtggttctgtatacctgccaggtgccaaccacttgtaaaggtcttgatattttcaattcttagactacctatactttggcagaagttatatttaatgtaagttgtctaaatataa

SEQ ID NO: 67 - DNA, 2666 bp, Artificial Sequence
  Synthetic Construct
gatccgccatcgtgggtgagtgttagctctgtggccgcgctctggctagtggcgctacgcgtcgctctcacgggtgtcgtcggatctaatccgtctcttttcgatagcaggtggagccgccgccacgatggaagggaagtggttgctgtgtatgttactggtgcttggaactgctattgttgaggctcatgatggacatgatgatgatgtgattgatattgaggatgaccttgacgatgtcattgaagaggtagaagactcaaaaccagataccactgctcctccttcatctcccaaggttacttacaaagctccagttccaacaggggaagtatattttgctgattcttttgacagaggaactctgtcagggtggattttatccaaagccaagaaagacgataccgatgatgaaattgccaaatatgatggaaagtgggaggtagaggaaatgaaggagtcaaagcttccaggtgataaaggacttgtgttgatgtctcgggccaagcatcatgccatctctgctaaactgaacaagcccttcctgtttgacaccaagcctctcattgttcagtatgaggttaatttccaaaatggaatagaatgtggtggtgcctatgtgaaactgctttctaaaacaccagaactcaacctggatcagttccatgacaagaccccttatacgattatgtttggtccagataaatgtggagaggactataaactgcacttcatcttccgacacaaaaaccccaaaacgggtatctatgaagaaaaacatgctaagaggccagatgcagatctgaagacctattttactgataagaaaacacatctttacacactaatcttgaatccagataatagttttgaaatactggttgaccaatctgtggtgaatagtggaaatctgctcaatgacatgactcctcctgtaaatccttcacgtgaaattgaggacccagaagaccggaagcccgaggattgggatgaaagaccaaaaatcccagatccagaagctgtcaagccagatgactgggatgaagatgcccctgctaagattccagatgaagaggccacaaaacccgaaggctggttagatgatgagcctgagtacgtacctgatccagacgcagagaaacctgaggattgggatgaagacatggatggagaatgggaggctcctcagattgccaaccctagatgtgagtcagctcctggatgtggtgtctggcagcgacctgtgattgacaaccccaattataaaggcaaatggaagcctcctatgattgacaatcccagttaccagggaatctggaaacccaggaaaataccaaatccagatttctttgaagatctggaacctttcagaatgactccttttagtgctattggtttggagctgtggtccatgacctctgacattttttttgacaactttatcatttgtgctgatcgaagaatagttgatgattgggccaatgatggatggggcctgaagaaagctgctgatggggctgctgagccaggcgttgtggggcagatgaacgaggcagctgaagagcgcccgtggctgtgggtagtctatattctaactgtagcccttcctgtgttcctggttatcctcttctgctgttctggaaagaaacagaccagtggtatggagtataagaaaactgatgcacctcaaccggatgtgaaggaagaggaagaagagaaggaagaggaaaaggacaagggagatgaggaggaggaaggagaagagaaacttgaagagaaacagaaaagtgatgctgaagaagatggtggcactgtcagtcaagaggaggaagacagaaaacctaaagcagaggaggatgaaattttgaacagatcaccaagaaacagaaagccacgaagagagctcgaggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtctagatgattgtgtatgcgttaataaaaagaaggaactcgta

SEQ ID NO: 68 - DNA, 2657 bp, Artificial Sequence
  Synthetic Construct
gatccgccatcgtgggtgagtgttagctctgtggccgcgctctggctagtggcgctacgcgtcgctctcacgggtgtcgtcggatctaatccgtctcttttcgatagcaggtggagccgccgccacgatggaagggaagtggttgctgtgtatgttactggtgcttggaactgctattgttgaggctcatgatggacatgatgatgatgtgattgatattgaggatgaccttgacgatgtcattgaagaggtagaagactcaaaaccagataccactgctcctccttcatctcccaaggttacttacaaagctccagttccaacaggggaagtatattttgctgattcttttgacagaggaactctgtcagggtggattttatccaaagccaagaaagacgataccgatgatgaaattgccaaatatgatggaaagtgggaggtagaggaaatgaaggagtcaaagcttccaggtgataaaggacttgtgttgatgtctcgggccaagcatcatgccatctctgctaaactgaacaagcccttcctgtttgacaccaagcctctcattgttcagtatgaggttaatttccaaaatggaatagaatgtggtggtgcctatgtgaaactgctttctaaaacaccagaactcaacctggatcagttccatgacaagaccccttatacgattatgtttggtccagataaatgtggagaggactataaactgcacttcatcttccgacacaaaaaccccaaaacgggtatctatgaagaaaaacatgctaagaggccagatgcagatctgaagacctattttactgataagaaaacacatctttacacactaatcttgaatccagataatagttttgaaatactggttgaccaatctgtggtgaatagtggaaatctgctcaatgacatgactcctcctgtaaatccttcacgtgaaattgaggacccagaagaccggaagcccgaggattgggatgaaagaccaaaaatcccagatccagaagctgtcaagccagatgactgggatgaagatgcccctgctaagattccagatgaagaggccacaaaacccgaaggctggttagatgatgagcctgagtacgtacctgatccagacgcagagaaacctgaggattgggatgaagacatggatggagaatgggaggctcctcagattgccaaccctagatgtgagtcagctcctggatgtggtgtctggcagcgacctgtgattgacaaccccaattataaaggcaaatggaagcctcctatgattgacaatcccagttaccagggaatctggaaacccaggaaaataccaaatccagatttctttgaagatctggaacctttcagaatgactccttttagtgctattggtttggagctgtggtccatgacctctgacattttttttgacaactttatcatttgtgctgatcgaagaatagttgatgattgggccaatgatggatggggcctgaagaaagctgctgatggggctgctgagccaggcgttgtggggcagatgaacgaggcagctgaagagcgcccgtggctgtgggtagtctatattctaactgtagcccttcctgtgttcctggttatcctcttctgctgttctggaaagaaacagaccagtggtatggagtataagaaaactgatgcacctcaaccggatgtgaaggaagaggaagaagagaaggaagaggaaaaggacaagggagatgaggaggaggaaggagaagagaaacttgaagagaaacagaaaagtgatgctgaagaagatggtggcactgtcagtcaagaggaggaagacagaaaacctaaagcagaggaggatgaaattttgaacagatcaccaagaaacagaaagccacgaagagagctcgaggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtctagatgattgtgtatgcgttaataaaaagaaggaactcgta

SEQ ID NO: 69 - DNA, 989 bp, Artificial Sequence
  Synthetic Construct
gatccgccatcgtgggtgagtgttagctctgtggccgcgctctggctagtggcgctacgcgtcgctctcacgggtgtcgtcggatctaatccgtctcttttcgatagcaggtggagccgccgccacgatggtgggacggaacagcgccatcgctgcaggagtgtgcggtgccctcttcatagggtactgcatctactttgaccgcaaaaggaggagtgaccccaacctcgaggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtctagatgattgtgtatgcgttaataaaaagaaggaactcgta

SEQ ID NO: 70 - DNA, 980 bp, Artificial Sequence
  Synthetic Construct
gatccgccatcgtgggtgagtgttagctctgtggccgcgctctggctagtggcgctacgcgtcgctctcacgggtgtcgtcggatctaatccgtctcttttcgatagcaggtggagccgccgccacgatggtgggacggaacagcgccatcgctgcaggagtgtgcggtgccctcttcatagggtactgcatctactttgaccgcaaaaggaggagtgaccccaacctcgaggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtctagatgattgtgtatgcgttaataaaaagaaggaactcgta

SEQ ID NO: 71 - DNA, 1895 bp, Artificial Sequence
  Synthetic Construct
gatccgccatcgtgggtgagtgttagctctgtggccgcgctctggctagtggcgctacgcgtcgctctcacgggtgtcgtcggatctaatccgtctcttttcgatagcaggtggagccgccgccacgatgtggtggtccttgatccttctttcttgcctgctggcactgaccagtgcccatgacaagccttccttccacccgctgtcggatgacctgattaactatatcaacaaacagaatacaacatggcaggctggacgcaacttctacaatgttgacataagctatctgaagaagctgtgtggcactgtcctgggtggacccaaactgccaggaagggttgcgttcggtgaggacatagatctacctgaaacctttgatgcacgggaacaatggtccaactgcccgaccattggacagattagagaccagggctcctgcggctcttgttgggcatttggggcagtggaagccatttctgaccgaacctgcattcacaccaatggccgagtcaacgtggaggtgtctgctgaagacctgcttacttgctgtggtatccagtgtggggacggctgtaatggtggctatccctctggagcatggagcttctggacaaaaaaaggcctggtttcaggtggagtctacaattctcatgtaggctgcttaccatacaccatccctccctgcgagcaccatgtcaatggctcccgtcccccatgcactggagaaggagatactcccaggtgcaacaagagctgtgaagctggctactccccatcctacaaagaggataagcactttgggtacacttcctacagcgtgtctaacagtgtgaaggagatcatggcagaaatctacaaaaatggcccagtggagggtgccttcactgtgttttctgacttcttgacttacaaatcaggagtatacaagcatgaagccggtgatatgatgggtggccacgccatccgcatcctgggctggggagtagagaatggagttccctactggctggcagccaactcttggaaccttgactggggtgataatggcttctttaaaatcctcagaggagaaaaccactgtggcattgaatcagaaattgtggctggaatcccacgcactgaccagtactggggaagattcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgattgtgtatgcgttaataaaaagaaggaactcgta

SEQ ID NO: 72 - DNA, 1886 bp, Artificial Sequence
  Synthetic Construct
gatccgccatcgtgggtgagtgttagctctgtggccgcgctctggctagtggcgctacgcgtcgctctcacgggtgtcgtcggatctaatccgtctcttttcgatagcaggtggagccgccgccacgatgtggtggtccttgatccttctttcttgcctgctggcactgaccagtgcccatgacaagccttccttccacccgctgtcggatgacctgattaactatatcaacaaacagaatacaacatggcaggctggacgcaacttctacaatgttgacataagctatctgaagaagctgtgtggcactgtcctgggtggacccaaactgccaggaagggttgcgttcggtgaggacatagatctacctgaaacctttgatgcacgggaacaatggtccaactgcccgaccattggacagattagagaccagggctcctgcggctcttgttgggcatttggggcagtggaagccatttctgaccgaacctgcattcacaccaatggccgagtcaacgtggaggtgtctgctgaagacctgcttacttgctgtggtatccagtgtggggacggctgtaatggtggctatccctctggagcatggagcttctggacaaaaaaaggcctggtttcaggtggagtctacaattctcatgtaggctgcttaccatacaccatccctccctgcgagcaccatgtcaatggctcccgtcccccatgcactggagaaggagatactcccaggtgcaacaagagctgtgaagctggctactccccatcctacaaagaggataagcactttgggtacacttcctacagcgtgtctaacagtgtgaaggagatcatggcagaaatctacaaaaatggcccagtggagggtgccttcactgtgttttctgacttcttgacttacaaatcaggagtatacaagcatgaagccggtgatatgatgggtggccacgccatccgcatcctgggctggggagtagagaatggagttccctactggctggcagccaactcttggaaccttgactggggtgataatggcttctttaaaatcctcagaggagaaaaccactgtggcattgaatcagaaattgtggctggaatcccacgcactgaccagtactggggaagattcgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgattgtgtatgcgttaataaaaagaaggaactcgta

SEQ ID NO: 73 - DNA, 985 bp, Artificial Sequence
  Synthetic Construct
gatccgccatcgtgggtgagtgttagctctgtggccgcgctctggctagtggcgctacgcgtcgctctcacgggtgtcgtcggatctaatccgtctcttttcgatagcaggtggagccgccgccacgatggccccaaagaagaagcggaaggtcggtatccacggagtcccagcagccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcgtcctgctgctactaagaaagctggtcaagctaagaaaaagaaataagcggccgcttgtgtatgcgttaataaaaagaaggaactcgta

SEQ ID NO: 74 - DNA, 979 bp, Artificial Sequence
  Synthetic Construct
ggggatccgccatcgtgggtgagtgttagctctgtggccgcgctctggctagtggcgctacgcgtcgctctcacgggtgtcgtcggatctaatccgtctcttttcgatagcaggtggagccgccgccacgatggccccaaagaagaagcggaaggtcggtatccacggagtcccagcagccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagaagcgtcctgctgctactaagaaagctggtcaagctaagaaaaagaaataagcggccgcttgtgtatgcgttaataaaaagaaggaactcgta

SEQ ID NO: 75 - DNA, 3530 bp, Artificial Sequence
  Synthetic Construct
gatccgccatcgtgggtgagtgttagctctgtggccgcgctctggctagtggcgctacgcgtcgctctcacgggtgtcgtcggatctaatccgtctcttttcgatagcaggtggagccgccgccacgatggtgggacggaacagcgccatcgctgcaggagtgtgcggtgccctcttcatagggtactgcatctactttgaccgcaaaaggaggagtgaccccaacctcgaggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggatccggcgcaacaaacttctctctgctgaaacaagccggagatgtcgaagagaatcctggaccgatggaagggaagtggttgctgtgtatgttactggtgcttggaactgctattgttgaggctcatgatggacatgatgatgatgtgattgatattgaggatgaccttgacgatgtcattgaagaggtagaagactcaaaaccagataccactgctcctccttcatctcccaaggttacttacaaagctccagttccaacaggggaagtatattttgctgattcttttgacagaggaactctgtcagggtggattttatccaaagccaagaaagacgataccgatgatgaaattgccaaatatgatggaaagtgggaggtagaggaaatgaaggagtcaaagcttccaggtgataaaggacttgtgttgatgtctcgggccaagcatcatgccatctctgctaaactgaacaagcccttcctgtttgacaccaagcctctcattgttcagtatgaggttaatttccaaaatggaatagaatgtggtggtgcctatgtgaaactgctttctaaaacaccagaactcaacctggatcagttccatgacaagaccccttatacgattatgtttggtccagataaatgtggagaggactataaactgcacttcatcttccgacacaaaaaccccaaaacgggtatctatgaagaaaaacatgctaagaggccagatgcagatctgaagacctattttactgataagaaaacacatctttacacactaatcttgaatccagataatagttttgaaatactggttgaccaatctgtggtgaatagtggaaatctgctcaatgacatgactcctcctgtaaatccttcacgtgaaattgaggacccagaagaccggaagcccgaggattgggatgaaagaccaaaaatcccagatccagaagctgtcaagccagatgactgggatgaagatgcccctgctaagattccagatgaagaggccacaaaacccgaaggctggttagatgatgagcctgagtacgtacctgatccagacgcagagaaacctgaggattgggatgaagacatggatggagaatgggaggctcctcagattgccaaccctagatgtgagtcagctcctggatgtggtgtctggcagcgacctgtgattgacaaccccaattataaaggcaaatggaagcctcctatgattgacaatcccagttaccagggaatctggaaacccaggaaaataccaaatccagatttctttgaagatctggaacctttcagaatgactccttttagtgctattggtttggagctgtggtccatgacctctgacattttttttgacaactttatcatttgtgctgatcgaagaatagttgatgattgggccaatgatggatggggcctgaagaaagctgctgatggggctgctgagccaggcgttgtggggcagatgaacgaggcagctgaagagcgcccgtggctgtgggtagtctatattctaactgtagcccttcctgtgttcctggttatcctcttctgctgttctggaaagaaacagaccagtggtatggagtataagaaaactgatgcacctcaaccggatgtgaaggaagaggaagaagagaaggaagaggaaaaggacaagggagatgaggaggaggaaggagaagagaaacttgaagagaaacagaaaagtgatgctgaagaagatggtggcactgtcagtcaagaggaggaagacagaaaacctaaagcagaggaggatgaaattttgaacagatcaccaagaaacagaaagccacgaagagaggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgattgtgtatgcgttaataaaaagaaggaactcgta

SEQ ID NO: 76 - DNA, 2771 bp, Artificial Sequence
  Synthetic Construct
gatccgccatcgtgggtgagtgttagctctgtggccgcgctctggctagtggcgctacgcgtcgctctcacgggtgtcgtcggatctaatccgtctcttttcgatagcaggtggagccgccgccacgatggtgggacggaacagcgccatcgctgcaggagtgtgcggtgccctcttcatagggtactgcatctactttgaccgcaaaaggaggagtgaccccaacctcgaggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggatccggcgcaacaaacttctctctgctgaaacaagccggagatgtcgaagagaatcctggaccgatgtggtggtccttgatccttctttcttgcctgctggcactgaccagtgcccatgacaagccttccttccacccgctgtcggatgacctgattaactatatcaacaaacagaatacaacatggcaggctggacgcaacttctacaatgttgacataagctatctgaagaagctgtgtggcactgtcctgggtggacccaaactgccaggaagggttgcgttcggtgaggacatagatctacctgaaacctttgatgcacgggaacaatggtccaactgcccgaccattggacagattagagaccagggctcctgcggctcttgttgggcatttggggcagtggaagccatttctgaccgaacctgcattcacaccaatggccgagtcaacgtggaggtgtctgctgaagacctgcttacttgctgtggtatccagtgtggggacggctgtaatggtggctatccctctggagcatggagcttctggacaaaaaaaggcctggtttcaggtggagtctacaattctcatgtaggctgcttaccatacaccatccctccctgcgagcaccatgtcaatggctcccgtcccccatgcactggagaaggagatactcccaggtgcaacaagagctgtgaagctggctactccccatcctacaaagaggataagcactttgggtacacttcctacagcgtgtctaacagtgtgaaggagatcatggcagaaatctacaaaaatggcccagtggagggtgccttcactgtgttttctgacttcttgacttacaaatcaggagtatacaagcatgaagccggtgatatgatgggtggccacgccatccgcatcctgggctggggagtagagaatggagttccctactggctggcagccaactcttggaaccttgactggggtgataatggcttctttaaaatcctcagaggagaaaaccactgtggcattgaatcagaaattgtggctggaatcccacgcactgaccagtactggggaagattcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgattgtgtatgcgttaataaaaagaaggaactcgta

SEQ ID NO: 77 - DNA, 1861 bp, Artificial Sequence
  Synthetic Construct
gatccgccatcgtgggtgagtgttagctctgtggccgcgctctggctagtggcgctacgcgtcgctctcacgggtgtcgtcggatctaatccgtctcttttcgatagcaggtggagccgccgccacgatggtgggacggaacagcgccatcgctgcaggagtgtgcggtgccctcttcatagggtactgcatctactttgaccgcaaaaggaggagtgaccccaacctcgaggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggatccggcgcaacaaacttctctctgctgaaacaagccggagatgtcgaagagaatcctggaccgatggccccaaagaagaagcggaaggtcggtatccacggagtcccagcagccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcgtcctgctgctactaagaaagctggtcaagctaagaaaaagaaataagcggccgcttgtgtatgcgttaataaaaagaaggaactcgta

SEQ ID NO: 78 - DNA, 3524 bp, Artificial Sequence
  Synthetic Construct
gatccgccatcgtgggtgagtgttagctctgtggccgcgctctggctagtggcgctacgcgtcgctctcacgggtgtcgtcggatctaatccgtctcttttcgatagcaggtggagccgccgccacgatggtgggacggaacagcgccatcgctgcaggagtgtgcggtgccctcttcatagggtactgcatctactttgaccgcaaaaggaggagtgaccccaacctcgaggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggaggtggaggcagcggaggcgggggcagtggaggagggggttccggtggtggtggtagtatggaagggaagtggttgctgtgtatgttactggtgcttggaactgctattgttgaggctcatgatggacatgatgatgatgtgattgatattgaggatgaccttgacgatgtcattgaagaggtagaagactcaaaaccagataccactgctcctccttcatctcccaaggttacttacaaagctccagttccaacaggggaagtatattttgctgattcttttgacagaggaactctgtcagggtggattttatccaaagccaagaaagacgataccgatgatgaaattgccaaatatgatggaaagtgggaggtagaggaaatgaaggagtcaaagcttccaggtgataaaggacttgtgttgatgtctcgggccaagcatcatgccatctctgctaaactgaacaagcccttcctgtttgacaccaagcctctcattgttcagtatgaggttaatttccaaaatggaatagaatgtggtggtgcctatgtgaaactgctttctaaaacaccagaactcaacctggatcagttccatgacaagaccccttatacgattatgtttggtccagataaatgtggagaggactataaactgcacttcatcttccgacacaaaaaccccaaaacgggtatctatgaagaaaaacatgctaagaggccagatgcagatctgaagacctattttactgataagaaaacacatctttacacactaatcttgaatccagataatagttttgaaatactggttgaccaatctgtggtgaatagtggaaatctgctcaatgacatgactcctcctgtaaatccttcacgtgaaattgaggacccagaagaccggaagcccgaggattgggatgaaagaccaaaaatcccagatccagaagctgtcaagccagatgactgggatgaagatgcccctgctaagattccagatgaagaggccacaaaacccgaaggctggttagatgatgagcctgagtacgtacctgatccagacgcagagaaacctgaggattgggatgaagacatggatggagaatgggaggctcctcagattgccaaccctagatgtgagtcagctcctggatgtggtgtctggcagcgacctgtgattgacaaccccaattataaaggcaaatggaagcctcctatgattgacaatcccagttaccagggaatctggaaacccaggaaaataccaaatccagatttctttgaagatctggaacctttcagaatgactccttttagtgctattggtttggagctgtggtccatgacctctgacattttttttgacaactttatcatttgtgctgatcgaagaatagttgatgattgggccaatgatggatggggcctgaagaaagctgctgatggggctgctgagccaggcgttgtggggcagatgaacgaggcagctgaagagcgcccgtggctgtgggtagtctatattctaactgtagcccttcctgtgttcctggttatcctcttctgctgttctggaaagaaacagaccagtggtatggagtataagaaaactgatgcacctcaaccggatgtgaaggaagaggaagaagagaaggaagaggaaaaggacaagggagatgaggaggaggaaggagaagagaaacttgaagagaaacagaaaagtgatgctgaagaagatggtggcactgtcagtcaagaggaggaagacagaaaacctaaagcagaggaggatgaaattttgaacagatcaccaagaaacagaaagccacgaagagaggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgattgtgtatgcgttaataaaaagaaggaactcgta

SEQ ID NO: 79 - DNA, 2765 bp, Artificial Sequence
  Synthetic Construct
gatccgccatcgtgggtgagtgttagctctgtggccgcgctctggctagtggcgctacgcgtcgctctcacgggtgtcgtcggatctaatccgtctcttttcgatagcaggtggagccgccgccacgatggtgggacggaacagcgccatcgctgcaggagtgtgcggtgccctcttcatagggtactgcatctactttgaccgcaaaaggaggagtgaccccaacctcgaggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggaggtggaggcagcggaggcgggggcagtggaggagggggttccggtggtggtggtagtatgtggtggtccttgatccttctttcttgcctgctggcactgaccagtgcccatgacaagccttccttccacccgctgtcggatgacctgattaactatatcaacaaacagaatacaacatggcaggctggacgcaacttctacaatgttgacataagctatctgaagaagctgtgtggcactgtcctgggtggacccaaactgccaggaagggttgcgttcggtgaggacatagatctacctgaaacctttgatgcacgggaacaatggtccaactgcccgaccattggacagattagagaccagggctcctgcggctcttgttgggcatttggggcagtggaagccatttctgaccgaacctgcattcacaccaatggccgagtcaacgtggaggtgtctgctgaagacctgcttacttgctgtggtatccagtgtggggacggctgtaatggtggctatccctctggagcatggagcttctggacaaaaaaaggcctggtttcaggtggagtctacaattctcatgtaggctgcttaccatacaccatccctccctgcgagcaccatgtcaatggctcccgtcccccatgcactggagaaggagatactcccaggtgcaacaagagctgtgaagctggctactccccatcctacaaagaggataagcactttgggtacacttcctacagcgtgtctaacagtgtgaaggagatcatggcagaaatctacaaaaatggcccagtggagggtgccttcactgtgttttctgacttcttgacttacaaatcaggagtatacaagcatgaagccggtgatatgatgggtggccacgccatccgcatcctgggctggggagtagagaatggagttccctactggctggcagccaactcttggaaccttgactggggtgataatggcttctttaaaatcctcagaggagaaaaccactgtggcattgaatcagaaattgtggctggaatcccacgcactgaccagtactggggaagattcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgattgtgtatgcgttaataaaaagaaggaactcgta

SEQ ID NO: 80 - DNA, 1855 bp, Artificial Sequence
  Synthetic Construct
gatccgccatcgtgggtgagtgttagctctgtggccgcgctctggctagtggcgctacgcgtcgctctcacgggtgtcgtcggatctaatccgtctcttttcgatagcaggtggagccgccgccacgatggtgggacggaacagcgccatcgctgcaggagtgtgcggtgccctcttcatagggtactgcatctactttgaccgcaaaaggaggagtgaccccaacctcgaggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggaggtggaggcagcggaggcgggggcagtggaggagggggttccggtggtggtggtagtatggccccaaagaagaagcggaaggtcggtatccacggagtcccagcagccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcgtcctgctgctactaagaaagctggtcaagctaagaaaaagaaataagcggccgcttgtgtatgcgttaataaaaagaaggaactcgta

SEQ ID NO: 81 - RNA, 90 nt, Artificial Sequence
  Synthetic Construct
ggggagaagagggaacaggacacaagagauaaacauaaacauaaacgacaagaaacacauacaaaagaaacaggacagaaaacagccacc

SEQ ID NO: 82 - RNA, 70 nt, Artificial Sequence
  Synthetic Construct
ggaaacacaauaacauaaucauacuacacaacuaacacauacaucacauacacaucacauaacagccacc

SEQ ID NO: 83 - RNA, 70 nt, Artificial Sequence
  Synthetic Construct
ggcuacacacucucacucucaucacucacuacucacucucucaucacucucacaucacaucacugccacc

SEQ ID NO: 84 - RNA, 70 nt, Artificial Sequence
  Synthetic Construct
ggcaaaaaucaaaaucaaucaucaucacaacaucaacaaucaaucaucaacacaucaucaagacaccacc

SEQ ID NO: 85 - RNA, 70 nt, Artificial Sequence
  Synthetic Construct
ggaagagaucaaaagcaacaaaucaaacagagaaacaauuagaacaagaaacagaagacaacaagccacc

SEQ ID NO: 86 - RNA, 70 nt, Artificial Sequence
  Synthetic Construct
ggcaucacacucucacucucaucucaacacuccuccucauuccaaucucucacacaucccauuagccacc

SEQ ID NO: 87 - RNA, 122 nt, Artificial Sequence
  Synthetic Construct
uuguguaugcguuaauaaaaagaaggaacucguaaaaacucaauguauuucugaggaagcguggugcauaaugccacgcagcgucugcauaacuuuuauuauuucuuuuauuaaucaacaaa

SEQ ID NO: 88 - RNA, 88 nt, Artificial Sequence
  Synthetic Construct
aaaacucaauguauuucugaggaagcguggugcauaaugccacgcagcgucugcauaacuuuuauuauuucuuuuauuaaucaacaaa

SEQ ID NO: 89 - RNA, 92 nt, Artificial Sequence
  Synthetic Construct
uuguguaugcguuaauaaaaagaaggaacucguauaugucuguuuuuguaucuuuaugcuguauuuuaacacuuuguauuacuuagguuauu

SEQ ID NO: 90 - RNA, 58 nt, Artificial Sequence
  Synthetic Construct
uaugucuguuuuuguaucuuuaugcuguauuuuaacacuuuguauuacuuagguuauu

SEQ ID NO: 91 - RNA, 74 nt, Artificial Sequence
  Synthetic Construct
uuguguaugcguuaauaaaaagaaggaacucguaaacuccaggacuguauuugugacuaauuguauaacagguu

SEQ ID NO: 92 - RNA, 40 nt, Artificial Sequence
  Synthetic Construct
aacuccaggacuguauuugugacuaauuguauaacagguu

SEQ ID NO: 93 - RNA, 4179 nt, Artificial Sequence
  Synthetic Construct
ggcaaaaaucaaaaucaaucaucaucacaacaucaacaaucaaucaucaacacaucaucaagacaccaccauggggguuaaggugcucuucgcgcucaucuguauugcuguggcggaagcaguuaaucuuacaaccagaacucaauuacccccugcauacacuaauucuuucacacgugguguuuauuacccugacaaaguuuucagauccucaguuuuacauucaacucaggacuuguucuuaccuuucuuuuccaauguuacuugguuccaugcuauacaugucucugggaccaaugguacuaagagguuugauaacccuguccuaccauuuaaugaugguguuuauuuugcuuccacugagaagucuaacauaauaagaggcuggauuuuugguacuacuuuagauucgaagacccagucccuacuuauuguuaauaacgcuacuaauguuguuauuaaagucugugaauuucaauuuuguaaugauccauuuuuggguguuuauuaccacaaaaacaacaaaaguuggauggaaagugaguucagaguuuauucuagugcgaauaauugcacuuuugaauaugucucucagccuuuucuuauggaccuugaaggaaaacaggguaauuucaaaaaucuuagggaauuuguguuuaagaauauugaugguuauuuuaaaauauauucuaagcacacgccuauuaauuuagugcgugaucucccucaggguuuuucggcuuuagaaccauugguagauuugccaauagguauuaacaucacuagguuucaaacuuuacuugcuuuacauagaaguuauuugacuccuggugauucuucuucagguuggacagcuggugcugcagcuuauuauguggguuaucuucaaccuaggacuuuucuauuaaaauauaaugaaaauggaaccauuacagaugcuguagacugugcacuugacccucucucagaaacaaaguguacguugaaauccuucacuguagaaaaaggaaucuaucaaacuucuaacuuuagaguccaaccaacagaaucuauuguuagauuuccuaauauuacaaacuugugcccuuuuggugaaguuuuuaacgccaccagauuugcaucuguuuaugcuuggaacaggaagagaaucagcaacuguguugcugauuauucuguccuauauaauuccgcaucauuuuccacuuuuaaguguuauggagugucuccuacuaaauuaaaugaucucugcuuuacuaaugucuaugcagauucauuuguaauuagaggugaugaagucagacaaaucgcuccagggcaaacuggaaagauugcugauuauaauuauaaauuaccagaugauuuuacaggcugcguuauagcuuggaauucuaacaaucuugauucuaagguuggugguaauuauaauuaccuguauagauuguuuaggaagucuaaucucaaaccuuuugagagagauauuucaacugaaaucuaucaggccgguagcacaccuuguaaugguguugaagguuuuaauuguuacuuuccuuuacaaucauaugguuuccaacccacuaaugguguugguuaccaaccauacagaguaguaguacuuucuuuugaacuucuacaugcaccagcaacuguuuguggaccuaaaaagucuacuaauuugguuaaaaacaaaugugucaauuucaacuucaaugguuuaacaggcacagguguucuuacugagucuaacaaaaaguuucugccuuuccaacaauuuggcagagacauugcugacacuacugaugcuguccgugauccacagacacuugagauucuugacauuacaccauguucuuuugguggugucaguguuauaacaccaggaacaaauacuucuaaccagguugcuguucuuuaucaggauguuaacugcacagaagucccuguugcuauucaugcagaucaacuuacuccuacuuggcguguuuauucuacagguucuaauguuuuucaaacacgugcaggcuguuuaauaggggcugaacaugucaacaacucauaugagugugacauacccauuggugcagguauaugcgcuaguuaucagacucagacuaauucuccucggcgggcacguaguguagcuagucaauccaucauugccuacacuaugucacuuggugcagaaaauucaguugcuuacucuaauaacucuauugccauacccacaaauuuuacuauuaguguuaccacagaaauucuaccagugucuaugaccaagacaucaguagauuguacaauguacauuuguggugauucaacugaaugcagcaaucuuuuguugcaauauggcaguuuuuguacacaauuaaaccgugcuuuaacuggaauagcuguugaacaagacaaaaacacccaagaaguuuuugcacaagucaaacaaauuuacaaaacaccaccaauuaaagauuuuggugguuuuaauuuuucacaaauauuaccagauccaucaaaaccaagcaagaggucauuuauugaagaucuacuuuucaacaaagugacacuugcagaugcuggcuucaucaaacaauauggugauugccuuggugauauugcugcuagagaccucauuugugcacaaaaguuuaacggccuuacuguuuugccaccuuugcucacagaugaaaugauugcucaauacacuucugcacuguuagcggguacaaucacuucugguuggaccuuuggugcaggugcugcauuacaaauaccauuugcuaugcaaauggcuuauagguuuaaugguauuggaguuacacagaauguucucuaugagaaccaaaaauugauugccaaccaauuuaauagugcuauuggcaaaauucaagacucacuuucuuccacagcaagugcacuuggaaaacuucaagauguggucaaccaaaaugcacaagcuuuaaacacgcuuguuaaacaacuuagcuccaauuuuggugcaauuucaaguguuuuaaaugauauccuuucacgucuugacaaaguugaggcugaagugcaaauugauagguugaucacaggcagacuucaaaguuugcagacauaugugacucaacaauuaauuagagcugcagaaaucagagcuucugcuaaucuugcugcuacuaaaaugucagaguguguacuuggacaaucaaaaagaguugauuuuuguggaaagggcuaucaucuuauguccuucccucagucagcaccucaugguguagucuucuugcaugugacuuaugucccugcacaagaaaagaacuucacaacugcuccugccauuugucaugauggaaaagcacacuuuccucgugaaggugucuuuguuucaaauggcacacacugguuuguaacacaaaggaauuuuuaugaaccacaaaucauuacuacagacaacacauuugugucugguaacugugauguuguaauaggaauugucaacaacacaguuuaugauccuuugcaaccugaauuagacucauucaaggaggaguuagauaaauauuuuaagaaucauacaucaccagauguugauuuaggugacaucucuggcauuaaugcuucaguuguaaacauucaaaaagaaauugaccgccucaaugagguugccaagaauuuaaaugaaucucucaucgaucuccaagaacuuggaaaguaugagcaguauauaaaauggccaugguacauuuggcuagguuuuauagcuggcuugauugccauaguaauggugacaauuaugcuuugcuguaugaccaguugcuguaguugucucaagggcuguuguucuuguggauccugcugcaaauuugaugaagacgacucugagccagugcucaaaggagucaaauuacauuacacaggcggcggagguucugauuacaaggacgaugaugauaaauaauuguguaugcguuaauaaaaagaaggaacucguaaaaacucaauguauuucugaggaagcguggugcauaaugccacgcagcgucugcauaacuuuuauuauuucuuuuauuaaucaacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 94 - RNA, 987 nt, Artificial Sequence
  Synthetic Construct
ggcaaaaaucaaaaucaaucaucaucacaacaucaacaaucaaucaucaacacaucaucaagacaccaccauggggguuaaggugcucuucgcgcucaucuguauugcuguggcggaagcaaauauuacaaacuugugcccuuuuggugaaguuuuuaacgccaccagauuugcaucuguuuaugcuuggaacaggaagagaaucagcaacuguguugcugauuauucuguccuauauaauuccgcaucauuuuccacuuuuaaguguuauggagugucuccuacuaaauuaaaugaucucugcuuuacuaaugucuaugcagauucauuuguaauuagaggugaugaagucagacaaaucgcuccagggcaaacuggaaagauugcugauuauaauuauaaauuaccagaugauuuuacaggcugcguuauagcuuggaauucuaacaaucuugauucuaagguuggugguaauuauaauuaccuguauagauuguuuaggaagucuaaucucaaaccuuuugagagagauauuucaacugaaaucuaucaggccgguagcacaccuuguaaugguguugaagguuuuaauuguuacuuuccuuuacaaucauaugguuuccaacccacuaaugguguugguuaccaaccauacagaguaguaguacuuucuuuugaacuucuacaugcaccagcaacuguuggcggcggagguucugauuacaaggacgaugaugauaaauaauuguguaugcguuaauaaaaagaaggaacucguaaaaacucaauguauuucugaggaagcguggugcauaaugccacgcagcgucugcauaacuuuuauuauuucuuuuauuaaucaacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 95 - RNA, 636 nt, Artificial Sequence
  Synthetic Construct
ggcaaaaaucaaaaucaaucaucaucacaacaucaacaaucaaucaucaacacaucaucaagacaccaccauggggguuaaggugcucuucgcgcucaucuguauugcuguggcggaagcauacucauucguuucggaagagacagguacguuaauaguuaauagcguacuucuuuuucuugcuuucgugguauucuugcuaguuacacuagccauccuuacugcgcuucgauugugugcguacugcugcaauauuguuaacgugagucuuguaaaaccuucuuuuuacguuuacucucguguuaaaaaucugaauucuucuagaguuccugaucuucuggucggcggaggagggucauacaccgacauagagaugaaucggcuuggcaaauaauuguguaugcguuaauaaaaagaaggaacucguaaaaacucaauguauuucugaggaagcguggugcauaaugccacgcagcgucugcauaacuuuuauuauuucuuuuauuaaucaacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 96 - RNA, 1077 nt, Artificial Sequence
  Synthetic Construct
ggcaaaaaucaaaaucaaucaucaucacaacaucaacaaucaaucaucaacacaucaucaagacaccaccauggggguuaaggugcucuucgcgcucaucuguauugcuguggcggaagcagcagauuccaacgguacuauuaccguugaagagcuuaaaaagcuccuugaacaauggaaccuaguaauagguuuccuauuccuuacauggauuugucuucuacaauuugccuaugccaacaggaauagguuuuuguauauaauuaaguuaauuuuccucuggcuguuauggccaguaacuuuagcuuguuuugugcuugcugcuguuuacagaauaaauuggaucaccgguggaauugcuaucgcaauggcuugucuuguaggcuugauguggcucagcuacuucauugcuucuuucagacuguuugcgcguacgcguuccauguggucauucaauccagaaacuaacauucuucucaacgugccacuccauggcacuauucugaccagaccgcuucuagaaagugaacucguaaucggagcugugauccuucguggacaucuucguauugcuggacaccaucuaggacgcugugacaucaaggaccugccuaaagaaaucacuguugcuacaucacgaacgcuuucuuauuacaaauugggagcuucgcagcguguagcaggugacucagguuuugcugcauacagucgcuacaggauuggcaacuauaaauuaaacacagaccauuccaguagcagugacaauauugcuuugcuuguacagggcggaggagggucauacaccgacauagagaugaaucggcuuggcaaauaauuguguaugcguuaauaaaaagaaggaacucguaaaaacucaauguauuucugaggaagcguggugcauaaugccacgcagcgucugcauaacuuuuauuauuucuuuuauuaaucaacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 97 - RNA, 1668 nt, Artificial Sequence
  Synthetic Construct
ggcaaaaaucaaaaucaaucaucaucacaacaucaacaaucaaucaucaacacaucaucaagacaccaccauggggguuaaggugcucuucgcgcucaucuguauugcuguggcggaagcaucugauaauggaccccaaaaucagcgaaaugcaccccgcauuacguuugguggacccucagauucaacuggcaguaaccagaauggagaacgcaguggggcgcgaucaaaacaacgucggccccaagguuuacccaauaauacugcgucuugguucaccgcucucacucaacauggcaaggaagaccuuaaauucccucgaggacaaggcguuccaauuaacaccaauagcaguccagaugaccaaauuggcuacuaccgaagagcuaccagacgaauucgugguggugacgguaaaaugaaagaucucaguccaagaugguauuucuacuaccuaggaacugggccagaagcuggacuucccuauggugcuaacaaagacggcaucauauggguugcaacugagggagccuugaauacaccaaaagaucacauuggcacccgcaauccugcuaacaaugcugcaaucgugcuacaacuuccucaaggaacaacauugccaaaaggcuucuacgcagaagggagcagaggcggcagucaagccucuucucguuccucaucacguagucgcaacaguucaagaaauucaacuccaggcagcaguaggggaacuucuccugcuagaauggcuggcaauggcggugaugcugcucuugcuuugcugcugcuugacagauugaaccagcuugagagcaaaaugucugguaaaggccaacaacaacaaggccaaacugucacuaagaaaucugcugcugaggcuucuaagaagccucggcaaaaacguacugccacuaaagcauacaauguaacacaagcuuucggcagacgugguccagaacaaacccaaggaaauuuuggggaccaggaacuaaucagacaaggaacugauuacaaacauuggccgcaaauugcacaauuugcccccagcgcuucagcguucuucggaaugucgcgcauuggcauggaagucacaccuucgggaacgugguugaccuacacaggugccaucaaauuggaugacaaagauccaaauuucaaagaucaagucauuuugcugaauaagcauauugacgcauacaaaacauucccaccaacagagccuaaaaaggacaaaaagaagaaggcugaugaaacucaagccuuaccgcagagacagaagaaacagcaaacugugacucuucuuccugcugcagauuuggaugauuucuccaaacaauugcaacaauccaugagcagugcugacucaacucaggccggcggaggagggucauacaccgacauagagaugaaucggcuuggcaaauaauuguguaugcguuaauaaaaagaaggaacucguaaaaacucaauguauuucugaggaagcguggugcauaaugccacgcagcgucugcauaacuuuuauuauuucuuuuauuaaucaacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2022/0162618 A1
(43) Pub. Date: May 26, 2022

(54) ENGINEERED MRNA SEQUENCES AND USES THEREOF

(71) Applicant: Ohio State Innovation Foundation, Columbus, OH (US)

(72) Inventors: Yizhou DONG, Dublin, OH (US); Chunxi ZENG, Dublin, OH (US); Weiyu ZHAO, Columbus, OH (US)

(21) Appl. No.: 17/442,471

(22) PCT Filed: Mar. 25, 2020
(86) PCT No.: PCT/US20/24674

§ 371 Date: Sep. 23, 2021

Related U.S. Application Data
(60) Provisional application No. 62/823,215, filed on Mar. 25, 2019.

(51) Int. Cl. C12N 15/67; C07K 14/005; A61K 39/215

(52) U.S. Cl. CPC C12N 15/67; C07K 14/005; A61K 2039/53; C12N 2770/20034; A61K 39/215

(57) ABSTRACT

The present disclosure relates to a series of engineered mRNA sequences and methods of use for improving protein expression.

### CROSS-REFERENCE TO RELATED APPLICATIONS

This application claims the benefit of U.S. Provisional Application No. 62/823,215, filed Mar. 25, 2019, which is expressly incorporated herein by reference.

### STATEMENT REGARDING FEDERALLY SPONSORED RESEARCH

This invention was made with government support under Grant No. R35GM119679 awarded by the National Institutes of Health. The government has certain rights in the invention.

### FIELD

The present disclosure relates to a series of engineered mRNA sequences and methods of use for improving protein expression.

### BACKGROUND

Messenger RNAs (mRNAs) are important mediators and regulators of gene expression from DNA to protein. Proteins in all living organisms are produced intracellularly using mRNAs as blueprints in a process called translation. The intracellular process of making proteins from mRNAs is subjected to meticulous regulation in order to balance biological functions of various proteins.

Messenger RNA is a long polynucleotide chain which consists of several major segments from 5′ to 3′, namely, Cap, 5′ untranslated region (5′ UTR), coding region, 3′ untranslated region (3′ UTR) and tail. The cap at 5′ terminus is involved in recruitment of translation initiation complex including ribosome. Coding region dictates what protein will be produced upon translation. The 5′ UTR and 3′ UTR are critical elements that regulate expression level of the encoded protein from this mRNA. Their mechanisms of action rely heavily upon the interaction between their unique nucleotide sequences and corresponding RNA binding proteins (RBPs) that recognize these sequences. Half-life and expression efficacy of mRNA are commonly modulated by various RBPs that bind to 5′ and 3′ UTRs. Most mRNAs in mammalian cells contain polyadenosine (polyA) tails at their 3′ termini. PolyA tail contributes to stability of mRNA chain by conveying resistance to mRNA 3′-to-5′ decay pathway, therefore prolonging mRNA half-life. PolyA tail is also found to circle back to mRNA 5′ terminus and plays a role in translation initiation.

Many diseases arise from errors of cellular protein synthesis, resulting insufficient functional proteins or mutated detrimental ones. Traditional protein therapies manufacture desired proteins in other organisms and directly deliver them into cells to supplement or correct missing cellular functions. However, many delivered proteins are insufficient at low dose and immunogenic at high dose due to their exogenous nature.

An emerging field of mRNA therapeutics synthesizes protein-coding mRNAs in labs, through a process called in vitro transcription, and delivers mRNA into cells. The desired proteins encoded by the mRNAs can be produced by the intracellular protein synthesis machinery. However, the protein expression levels of the delivered mRNAs vary dramatically. What is needed are methods for improving the expression efficacy and half-life of delivered mRNAs.

### SUMMARY

Disclosed herein are a series of engineered mRNAs and methods for improving protein expression.

In some aspects, disclosed herein is an engineered mRNA comprising: a first nucleic acid sequence comprising an RPS27A 5′ untranslated region (5′UTR) sequence or an engineered 5′ untranslated region (5′UTR) sequence; a second nucleic acid sequence comprising a heterologous nucleic acid sequence; and a third nucleic acid sequence comprising an RPS27A 3′ untranslated region (3′UTR) sequence.

In some embodiments, the RPS27A 5′UTR sequence is selected from the group comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

In some embodiments, the heterologous nucleic acid sequence encodes a target protein. In some embodiments, the target protein is any protein of interest (POI).

In some embodiments, the target protein is an immunotherapeutic protein. In some embodiments, the target protein is a co-stimulatory molecule. In some embodiments, the target protein is a genome editing enzyme or a nuclease. In some embodiments, the target protein is for protein replacement therapy.

In some embodiments, the target protein comprises a fluorescent protein. In some embodiments, the target protein is fused to a fluorescent protein. In one embodiment, the fluorescent protein is mCherry (mCh). In some embodiments, the fluorescent protein is GFP or YFP.

In some embodiments, the target protein comprises a viral protein. In some embodiments, the viral protein is a COVID-19 protein.

In some embodiments, the RPS27A 3′UTR sequence is selected from the group comprising SEQ ID NO: 24, SEQ ID NO: 25, SEQ ID NO: 26, SEQ ID NO: 87, SEQ ID NO: 89, or SEQ ID NO: 91.

In some embodiments, the engineered mRNA of any preceding aspect comprises an RNA sequence selected from the group comprising SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, or SEQ ID NO: 40.

In some embodiments, the engineered mRNA of any preceding aspect comprises an RNA sequence selected from the group comprising SEQ ID NO: 93, SEQ ID NO: 94, SEQ ID NO: 95, SEQ ID NO: 96, or SEQ ID NO: 97.

In some embodiments, the mRNA comprises at least one chemically modified nucleotide. In some embodiments, the at least one chemically modified nucleotide is a chemically modified nucleobase. In some embodiments, the chemically modified nucleobase is pseudouridine.

In some aspects, disclosed herein is a vector comprising the engineered mRNA of any preceding aspect. In some embodiments, a cell comprises the vector of any preceding aspect.

In some aspects, disclosed herein is a method of increasing protein expression, comprising the steps: introducing into a cell an engineered mRNA, comprising: a first nucleic acid sequence comprising an RPS27A 5′UTR sequence; a second nucleic acid sequence comprising a heterologous nucleic acid sequence; and a third nucleic acid sequence comprising an RPS27A 3′UTR sequence.

In some embodiments, the RPS27A 5′UTR sequence is selected from the group comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

In some embodiments, the heterologous nucleic acid sequence encodes a target protein. In some embodiments, the target protein is any protein of interest (POI).

In some embodiments, the target protein comprises a fluorescent protein. In some embodiments, the target protein is fused to a fluorescent protein. In one embodiment, the fluorescent protein is mCherry (mCh). In some embodiments, the fluorescent protein is GFP or YFP.

In some embodiments, the target protein comprises a viral protein. In some embodiments, the viral protein is a COVID-19 protein.

In some embodiments, the RPS27A 3′UTR sequence is selected from the group comprising SEQ ID NO: 24, SEQ ID NO: 25, SEQ ID NO: 26, SEQ ID NO: 87, SEQ ID NO: 89, or SEQ ID NO: 91.

In some embodiments, the engineered mRNA comprises an RNA sequence selected from the group comprising SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, or SEQ ID NO: 40.

In some embodiments, the engineered mRNA comprises an RNA sequence selected from the group comprising SEQ ID NO: 93, SEQ ID NO: 94, SEQ ID NO: 95, SEQ ID NO: 96, or SEQ ID NO: 97.

In some aspects, disclosed herein is an engineered mRNA comprising: a first nucleic acid sequence comprising an engineered 5′ untranslated region (5′UTR) sequence; a second nucleic acid sequence comprising a heterologous nucleic acid sequence; and a third nucleic acid sequence comprising an RPS27A 3′ untranslated region (3′UTR) sequence.

In some embodiments, the engineered 5′UTR sequence is selected from the group comprising SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 81, SEQ ID NO: 82, SEQ ID NO: 83, SEQ ID NO: 84, SEQ ID NO: 85, or SEQ ID NO: 86.

### BRIEF DESCRIPTION OF THE DRAWINGS

The accompanying figures, which are incorporated in and constitute a part of this specification, illustrate several aspects described below.

FIGS. 1A-1B show in vitro expression of luciferase mRNAs with or without modified 5′UTR and 3′UTR from mouse ribosomal protein S27a gene in A549 (FIG. 1A) and Hep3B (FIG. 1B) cells. AG+G, AG+G w/o 3UTR and CYBA are control luciferase mRNAs with identical coding sequences as other engineered mRNAs.

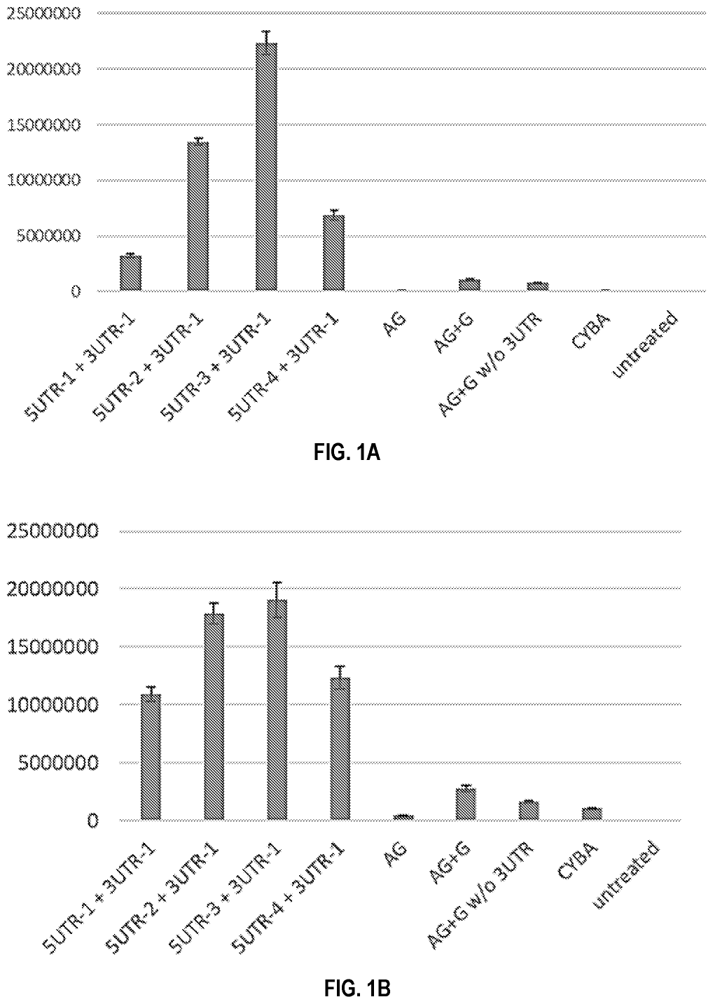

FIGS. 2A-2C show in vitro expression of eGFP mRNAs with or without modified 5′UTR and 3′UTR from mouse ribosomal protein S27a gene in A549 (FIG. 2A), Hep3B cells (FIG. 2B), and 293T cells (FIG. 2C).

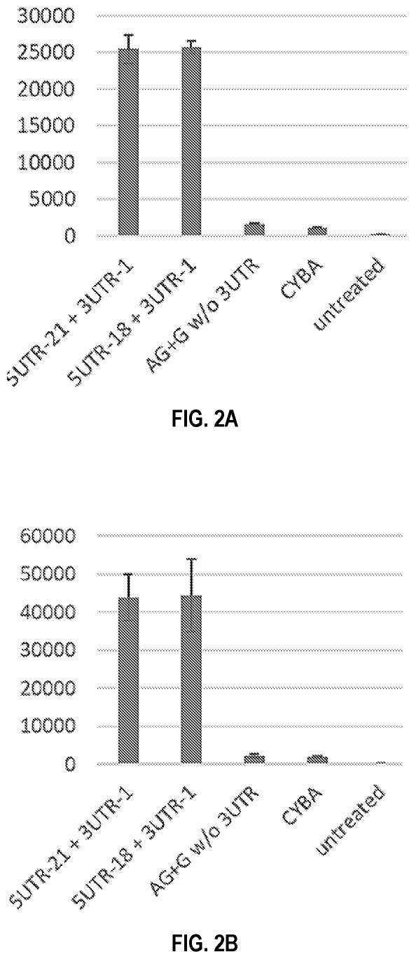

FIG. 3 shows in vitro expression of luciferase mRNA engineered with 5UTR-18 and 3UTR-1 with or without pseudouridine modification in A549 cells.

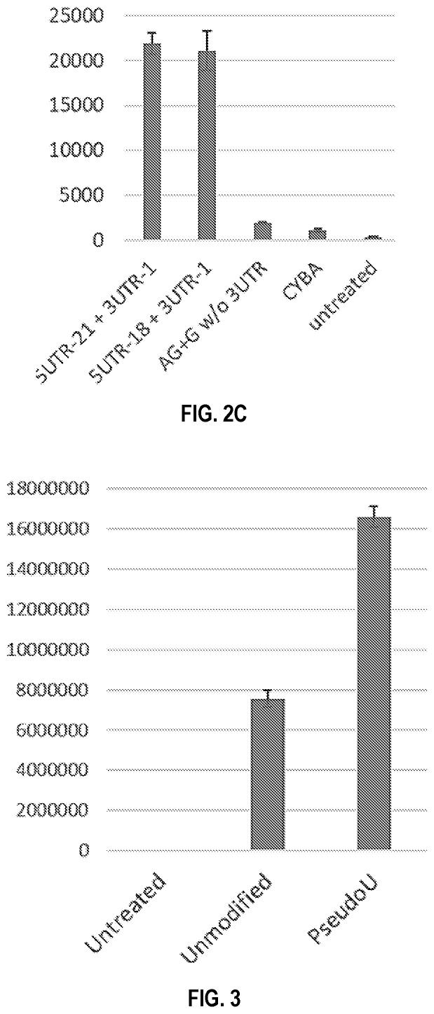

FIGS. 4A-4B show in vitro expression of pseudouridine modified luciferase mRNAs engineered with 5UTR-22+3UTR-1 and engineered with 5UTR-23+3UTR-1 in Hep3B (FIG. 4A) and A549 cells (FIG. 4B).

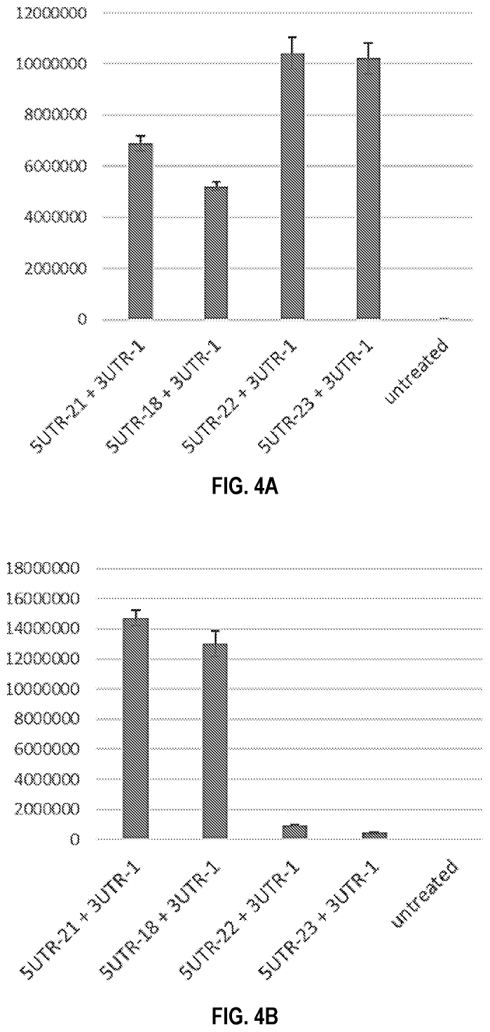

FIG. 5 shows live imaging of organelle targeting by eGFP/mCherry mRNA with 5′ UTR and 3′ UTR sequence disclosed herein or by commercially available imaging probes using live Hep3B cells.

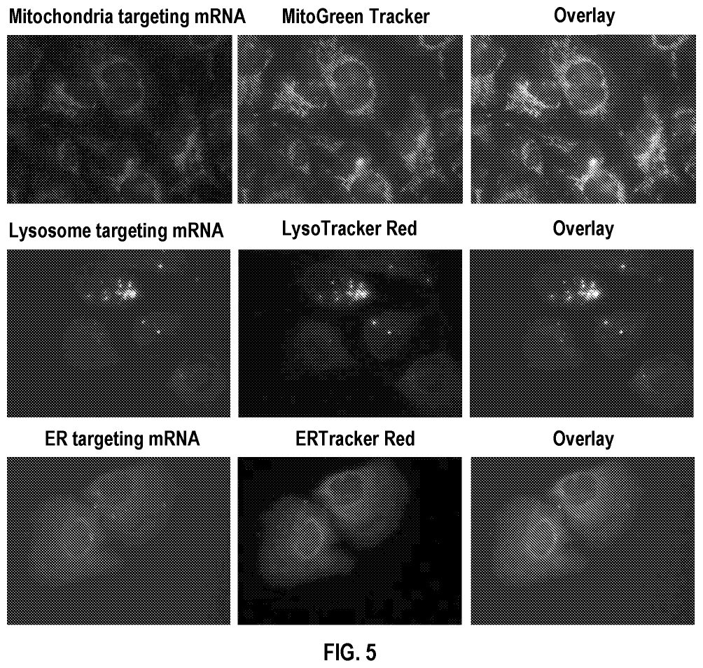

FIGS. 6A-6B show firefly luciferase mRNAs with 5′ UTR consisting of 10 nt (5UTR-12), 30 nt (5UTR-14), 50 nt (5UTR-16), 70 nt (5UTR-18), or 90 nt (5UTR-24) were tested for expression in mammalian cells. The results are shown for Hep3B cells (FIG. 6A) and 293T cells (FIG. 6B), respectively.

FIGS. 7A-7B show that the microRNA target sites located in 5′ UTR were removed to enhance mRNA expression. The results are shown for Hep3B cells (FIG. 7A) and 293T cells (FIG. 7B), respectively.

FIGS. 8A-8B show that additional functional RNA motifs were appended to the 3′ end of 3UTR-1 to enhance mRNA expression. The results are shown for Hep3B cells (FIG. 8A) and 293T cells (FIG. 8B), respectively.

### DETAILED DESCRIPTION

Disclosed herein are a series of engineered mRNAs comprising modified portions of the RPS27A 5′UTR and the RPS27A 3′UTR and methods for improving protein expression. Also disclosed herein are a series of engineered mRNAs comprising engineered (non-naturally occurring) 5′UTR sequences and methods for improving protein expression.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The term “target protein” refers to a protein or a polypeptide expressed by a given engineered mRNA. Target proteins may be naturally-occurring or man-made molecules. Also, they can be employed in their unaltered state or as aggregates with other species.

The term “complementary” refers to the topological compatibility or matching together of interacting surfaces of a probe molecule and its target. Thus, the target and its probe can be described as complementary, and furthermore, the contact surface characteristics are complementary to each other.

The term “hybridization” refers to a process of establishing a non-covalent, sequence-specific interaction between two or more complementary strands of nucleic acids into a single hybrid, which in the case of two strands is referred to as a duplex.

The term “anneal” refers to the process by which a single-stranded nucleic acid sequence pairs by hydrogen bonds to a complementary sequence, forming a double-stranded nucleic acid sequence, including the reformation (renaturation) of complementary strands that were separated by heat (thermally denatured).

The term “melting” refers to the denaturation of a double-stranded nucleic acid sequence due to high temperatures, resulting in the separation of the double strand into two single strands by breaking the hydrogen bonds between the strands.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc.

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom.

The term “expression cassette” or “vector” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc.

“Increase” can refer to any change that results in a higher level of gene expression, protein expression, amount of a symptom, disease, composition, condition, or activity. A substance is also understood to increase the level of the gene, the protein, the composition, or the amount of the condition when the level of the gene, the protein, the composition, or the amount of the condition is more/higher relative to the output of the level of the gene, the protein, the composition, or the amount of the condition without the substance. Also, for example, an increase can be a change in the symptoms of a disorder such that the symptoms are less than previously observed. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

“Decrease” can refer to any change that results in a lower level of gene expression, protein expression, amount of a symptom, disease, composition, condition, or activity. A substance is also understood to decrease the level of the gene, the protein, the composition, or the amount of the condition when the level of the gene, the protein, the composition, or the amount of the condition is less/lower relative to the output of the level of the gene, the protein, the composition, or the amount of the condition without the substance. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The phrase “codon optimized” as it refers to genes or coding regions of nucleic acid molecules for the transformation of various hosts, refers to the alteration of codons in the gene or coding regions of polynucleic acid molecules to reflect the typical codon usage of a selected organism without altering the polypeptide encoded by the DNA. Such optimization includes replacing at least one, or more than one, or a significant number, of codons with one or more codons that are more frequently used in the genes of that selected organism.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

As used throughout, by a “subject” (or a “host”) is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human.

The term “about” as used herein when referring to a measurable value such as an amount, a percentage, and the like, is meant to encompass variations of 20%, ±10%, +5%, or +1% from the measurable value.

A nucleic acid sequence is “heterologous” to a second nucleic acid sequence if it originates from a foreign species, or, if from the same species, is modified by human action from its original form. For example, a promoter operably linked to a heterologous coding sequence refers to a coding sequence from a species different from that from which the promoter was derived, or, if from the same species, a coding sequence which is different from naturally occurring allelic variants.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially. Prophylactic treatments are administered to a subject prior to onset, during early onset, or after an established development of cancer. Prophylactic administration can occur for several days to years prior to the manifestation of symptoms of an infection.

As used herein, the term “vaccine” refers to a formulation which contains the engineered mRNAs of the present invention, which is in a form that is capable of being administered to a subject and which induces a protective immune response sufficient to induce immunity to prevent and/or ameliorate an infection and/or to reduce at least one symptom of an infection and/or to enhance the efficacy of another dose of vaccines. Typically, the vaccine comprises a conventional saline or buffered aqueous solution medium in which the composition of the present invention is suspended or dissolved. In this form, the composition of the present invention can be used conveniently to prevent, ameliorate, or otherwise treat an infection. Upon introduction into a host, the vaccine is able to provoke an immune response including, but not limited to, the production of antibodies and/or cytokines and/or the activation of CD8+ T cells, antigen presenting cells, CD4+ T cells, dendritic cells and/or other cellular responses.

As used herein the term “adjuvant” refers to a compound that, when used in combination with a specific immunogen in a formulation, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses.

A “co-stimulatory molecule” refers to the cognate binding partner on an immune cell (e.g. T cell) that specifically binds with a co-stimulatory ligand, thereby mediating a co-stimulatory response by the T cell, such as, but not limited to, proliferation.

### Compositions and Methods

Disclosed herein are a series of engineered mRNAs and methods for improving protein expression. In some aspects, disclosed herein is an engineered mRNA comprising: a first nucleic acid sequence comprising an RPS27A 5′ untranslated region (5′UTR) sequence or an engineered 5′ untranslated region (5′UTR) sequence; a second nucleic acid sequence comprising a heterologous nucleic acid sequence; and a third nucleic acid sequence comprising an RPS27A (3′ untranslated region) 3′UTR sequence.

In some embodiments, the RPS27A 5′UTR sequence is selected from the group comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11. In some embodiments, the RPS27A 5′UTR sequence is SEQ ID NO: 1. In some embodiments, the RPS27A 5′UTR sequence is SEQ ID NO: 2. In some embodiments, the RPS27A 5′UTR sequence is SEQ ID NO: 3. In some embodiments, the RPS27A 5′UTR sequence is SEQ ID NO: 4. In some embodiments, the RPS27A 5′UTR sequence is SEQ ID NO: 5. In some embodiments, the RPS27A 5′UTR sequence is SEQ ID NO: 6. In some embodiments, the RPS27A 5′UTR sequence is SEQ ID NO: 7. In some embodiments, the RPS27A 5′UTR sequence is SEQ ID NO: 8. In some embodiments, the RPS27A 5′UTR sequence is SEQ ID NO: 9. In some embodiments, the RPS27A 5′UTR sequence is SEQ ID NO: 10. In some embodiments, the RPS27A 5′UTR sequence is SEQ ID NO: 11.

In some embodiments, the RPS27A 5′UTR sequence is selected from the group comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or a fragment or functionally active variant thereof.

In some embodiments, the RPS27A 5′UTR sequence is selected from the group comprising a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

In some embodiments, the RPS27A 3′UTR sequence is selected from the group comprising SEQ ID NO: 24, SEQ ID NO: 25, SEQ ID NO: 26, SEQ ID NO: 87, SEQ ID NO: 89, or SEQ ID NO: 91, or a fragment or functionally active variant thereof. In some embodiments, the RPS27A 3′UTR sequence is SEQ ID NO: 24. In some embodiments, the RPS27A 3′UTR sequence is SEQ ID NO: 25. In some embodiments, the RPS27A 3′UTR sequence is SEQ ID NO: 26. In some embodiments, the RPS27A 3′UTR sequence is SEQ ID NO: 87. In some embodiments, the RPS27A 3′UTR sequence is SEQ ID NO: 89. In some embodiments, the RPS27A 3′UTR sequence is SEQ ID NO: 91. In some embodiments, the RPS27A 3′UTR sequence of any preceding aspect comprises a functional motif A, motif B, and/or motif C, wherein the functional motif A comprises SEQ ID NO: 88, wherein the functional motif B comprises SEQ ID NO: 90, and wherein the functional motif C comprises SEQ ID NO: 92.

In some embodiments, the RPS27A 3′UTR sequence is selected from the group comprising a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 24, SEQ ID NO: 25, SEQ ID NO: 26, SEQ ID NO: 87, SEQ ID NO: 89, or SEQ ID NO: 91.

In some embodiments, the heterologous nucleic acid sequence encodes a target protein. The heterologous nucleic acid sequence or target protein can be any nucleic acid sequence/protein of interest.

In some embodiments, the target protein is an immunotherapeutic protein. In some embodiments, the target protein is a co-stimulatory molecule. In some embodiments, the target protein is a genome editing enzyme or a nuclease. In some embodiments, the target protein is for protein replacement therapy.

In some embodiments, the co-stimulatory molecule is selected from ICOS, CD28, CD27, HVEM, LIGHT, CD40L, 4-1BB, OX40, DR3, GITR, CD30, SLAM, CD2, CD226, Galectin9, TIM1, LFA1, B7-H2, B7-1, B7-2, CD70, LIGHT, HVEM, CD40, 4-1BBL, OX40L, TL1A, GITRL, CD30L, SLAM, CD48, CD58, CD155, CD112, CD80, CD86, ICOSL, TIM3, TIM4, ICAM1, or LFA3.

In some embodiments, the co-stimulatory molecule is ICOS. In some embodiments, the co-stimulatory molecule is CD28. In some embodiments, the co-stimulatory molecule is CD27. In some embodiments, the co-stimulatory molecule is HVEM. In some embodiments, the co-stimulatory molecule is LIGHT. In some embodiments, the co-stimulatory molecule is CD40L. In some embodiments, the co-stimulatory molecule is 4-1BB. In some embodiments, the co-stimulatory molecule is OX40. In some embodiments, the co-stimulatory molecule is DR2. In some embodiments, the co-stimulatory molecule is GITR. In some embodiments, the co-stimulatory molecule is CD30. In some embodiments, the co-stimulatory molecule is SLAM. In some embodiments, the co-stimulatory molecule is CD2. In some embodiments, the co-stimulatory molecule is CD226. In some embodiments, the co-stimulatory molecule is Galectin9. In some embodiments, the co-stimulatory molecule is TIM1. In some embodiments, the co-stimulatory molecule is LFA1. In some embodiments, the co-stimulatory molecule is B7-H2. In some embodiments, the co-stimulatory molecule is B7-1. In some embodiments, the co-stimulatory molecule is B7-2. In some embodiments, the co-stimulatory molecule is CD70. In some embodiments, the co-stimulatory molecule is LIGHT. In some embodiments, the co-stimulatory molecule is HVEM. In some embodiments, the co-stimulatory molecule is 4-1BBL. In some embodiments, the co-stimulatory molecule is OX40L. In some embodiments, the co-stimulatory molecule is TL1A. In some embodiments, the co-stimulatory molecule is GITRL. In some embodiments, the co-stimulatory molecule is CD30L. In some embodiments, the co-stimulatory molecule is CD48. In some embodiments, the co-stimulatory molecule is SLAM. In some embodiments, the co-stimulatory molecule is CD58. In some embodiments, the co-stimulatory molecule is CD155. In some embodiments, the co-stimulatory molecule is CD112. In some embodiments, the co-stimulatory molecule is CD80. In some embodiments, the co-stimulatory molecule is CD86. In some embodiments, the co-stimulatory molecule is ICOSL. In some embodiments, the co-stimulatory molecule is TIM3. In some embodiments, the co-stimulatory molecule is TIM4. In some embodiments, the co-stimulatory molecule is ICAM1. In some embodiments, the co-stimulatory molecule is LFA3.

The sequences for the co-stimulatory molecules include, for example (for human sequences): ICOS (NCBI Reference Sequence: NM_012092.3), CD28 (NCBI Reference Sequence: NM_006139.4), CD27 (NCBI Reference Sequence: NM_001242.4), HVEM (NCBI Reference Sequence: NM_003820.3), LIGHT (NCBI Reference Sequence: NM_003807.4), CD40L (NCBI Reference Sequence: NM_000074.2), 4-1BB (NCBI Reference Sequence: NM_001561.5), OX40 (NCBI Reference Sequence: NM_003327.4), DR3 (NCBI Reference Sequence: NM_148965.1), GITR (NCBI Reference Sequence: NM_004195.3), CD30 (GenBank: M83554.1), SLAM (NCBI Reference Sequence: NM_003037.4), CD2 (NCBI Reference Sequence: NM_001328609.1), CD226 (NCBI Reference Sequence: NM_006566.3), Galectin-9 (GenBank: AB040130.2), TIM1 (GenBank: U02082.1), B7-H2 (NCBI Reference Sequence: NM_015259.5), B7-1 (NCBI Reference Sequence: NM_005191.4), B7-2 (NCBI Reference Sequence: NM_175862.5), CD70 (NCBI Reference Sequence: NM_001252.5), CD40 (NCBI Reference Sequence: NM_001250.5), 4-1BBL (NCBI Reference Sequence: NM_003811.4), OX40L (NCBI Reference Sequence: NM_003326.5), TL1A (NCBI Reference Sequence: NM_005118.4), GITRL (GenBank: AY358868.1), CD30L (NCBI Reference Sequence: NM_001244.3), SLAM (GenBank: U33017.1), CD48 (NCBI Reference Sequence: NM_001778.4), CD58 (NCBI Reference Sequence: NM_001779.3), CD155 (NCBI Reference Sequence: NM_006505.5), CD 112 (NCBI Reference Sequence: NM_001042724.2), TIM3 (GenBank: AF450242.1), TIM4 (NCBI Reference Sequence: NM_138379.3), ICAM1 (NCBI Reference Sequence: NM_000201.3).

Accordingly, in some embodiments, the co-stimulatory molecule comprises a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to ICOS (NCBI Reference Sequence: NM_012092.3), CD28 (NCBI Reference Sequence: NM_006139.4), CD27 (NCBI Reference Sequence: NM_001242.4), HVEM (NCBI Reference Sequence: NM_003820.3), LIGHT (NCBI Reference Sequence: NM_003807.4), CD40L (NCBI Reference Sequence: NM_000074.2), 4-1BB (NCBI Reference Sequence: NM_001561.5), OX40 (NCBI Reference Sequence: NM_003327.4), DR3 (NCBI Reference Sequence: NM_148965.1), GITR (NCBI Reference Sequence: NM_004195.3), CD30 (GenBank: M83554.1), SLAM (NCBI Reference Sequence: NM_003037.4), CD2 (NCBI Reference Sequence: NM_001328609.1), CD226 (NCBI Reference Sequence: NM_006566.3), Galectin-9 (GenBank: AB040130.2), TIM1 (GenBank: U02082.1), B7-H2 (NCBI Reference Sequence: NM_015259.5), B7-1 (NCBI Reference Sequence: NM_005191.4), B7-2 (NCBI Reference Sequence: NM_175862.5), CD70 (NCBI Reference Sequence: NM_001252.5), CD40 (NCBI Reference Sequence: NM_001250.5), 4-1BBL (NCBI Reference Sequence: NM_003811.4), OX40L (NCBI Reference Sequence: NM_003326.5), TL1A (NCBI Reference Sequence: NM_005118.4), GITRL (GenBank: AY358868.1), CD30L (NCBI Reference Sequence: NM_001244.3), SLAM (GenBank: U33017.1), CD48 (NCBI Reference Sequence: NM_001778.4), CD58 (NCBI Reference Sequence: NM_001779.3), CD155 (NCBI Reference Sequence: NM_006505.5), CD112 (NCBI Reference Sequence: NM_001042724.2), TIM3 (GenBank: AF450242.1), TIM4 (NCBI Reference Sequence: NM_138379.3), ICAM1 (NCBI Reference Sequence: NM_000201.3), or a variant or a fragment thereof.

In some embodiments, the genome editing enzyme is selected from a zinc finger nuclease (ZFN), a transcription activator-like effector-based nuclease (TALEN), or a clustered regularly interspaced short palindromic repeats (CRISPR) system nuclease. In some embodiments, the genome editing enzyme is Cpf1, or a variant or homolog thereof. In some embodiments, the genome editing enzyme is Cas9, or a variant or homolog thereof.

In some embodiments, the target protein comprises a fluorescent protein. In some embodiments, the target protein is fused to a fluorescent protein. In one embodiment, the fluorescent protein comprises mCherry (mCh). In some embodiments, the fluorescent protein comprises GFP. In some embodiments, the fluorescent protein comprises YFP.

In some embodiments, the target protein comprises a viral protein. In some embodiments, the viral protein is a coronavirus protein. Coronaviruses constitute the subfamily Orthocoronavirinae, in the family Coronaviridae, order Nidovirales, and realm Riboviria.

They are enveloped viruses with a positive-sense single-stranded RNA genome and a nucleocapsid of helical symmetry. The genome size of coronaviruses ranges from approximately 27 to 34 kilobases. The structure of coronavirus generally consists of the following: spike protein, hemagglutinin-esterease dimer (HE), a membrane glycoprotein (M), an envelope protein (E) a nucleoclapid protein (N) and RNA. The coronavirus family comprises genera including, for example, alphacoronavius (e.g., Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512), betacoronavirus (e.g., COVID-19, Betacoronavirus 1, Human coronavirus HKU1, Murine coronavirus, Pipistrellus bat coronavirus HKU5, Rousettus bat coronavirus HKU9, Severe acute respiratory syndrome-related coronavirus, Tylonycteris bat coronavirus HKU4, Middle East respiratory syndrome-related coronavirus (MERS), Human coronavirus OC43, Hedgehog coronavirus 1 (EriCoV)), gammacoronavirus (e.g., Beluga whale coronavirus SW1, Infectious bronchitis virus), and deltacoronavirus (e.g., Bulbul coronavirus HKU11, Porcine coronavirus HKU15). In some embodiments, the viral protein is a protein of Severe acute respiratory syndrome-related coronavirus. In some embodiments, the viral protein is a protein of MERS coronavirus.

In some embodiments, the viral protein is a COVID-19 protein, including, for example, COVID-19 spike protein, COVID-19 envelope protein, COVID-19 membrane protein, or COVID-19 nucleocapsid protein, or a fragment thereof. In some embodiments, the viral protein is a receptor binding domain of a COVID-19 spike protein.

In some embodiments, the target protein is Factor IX. Factor IX is a human protein that is produced as a zymogen, an inactive precursor (accession number: HGNC: 3551; Entrez Gene: 2158; Ensembl: ENSG00000101981; OMIM: 300746 UniProtKB: P00740). In some embodiments, the target protein is phenylalanine hydroxylase (Accession number: HGNC: 8582; Entrez Gene: 5053; Ensembl: ENSG00000171759; OMIM: 612349; UniProtKB: P00439). In some embodiments, the target protein is CFTR. Other target proteins can include, but are not limited to, enzymes, enzyme cofactors, hormones, blood clotting factors, cytokines, growth factors, etc. See for example, U.S. Pat. No. 10,071,114, which is herein incorporated by reference.

In some embodiments, the RPS27A 5′UTR sequence comprises SEQ ID NO: 2 and the RPS27A 3′UTR sequence comprises SEQ ID NO: 24. In some embodiments, the RPS27A 5′UTR sequence comprises SEQ ID NO: 3 and the RPS27A 3′UTR sequence comprises SEQ ID NO: 24. In some embodiments, the RPS27A 5′UTR sequence comprises SEQ ID NO: 84 and the RPS27A 3′UTR sequence comprises SEQ ID NO: 87.

In some embodiments, the engineered mRNA of any preceding aspect further comprises a 120A tail.

In some embodiments, the engineered mRNA of any preceding aspect comprises an RNA sequence selected from the group comprising SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, or SEQ ID NO: 40.

In some embodiments, the engineered mRNA of any preceding aspect comprises an RNA sequence selected from the group comprising SEQ ID NO: 93, SEQ ID NO: 94, SEQ ID NO: 95, SEQ ID NO: 96, or SEQ ID NO: 97.

In some embodiments, the RPS27A 5′UTR sequence is a fragment of the endogenous (wild-type) RPS27A gene sequence. In some embodiments, the RPS27A 5′UTR sequence is a modified version of the RPS27A gene sequence (for example, comprises nucleotide changes, insertions, deletions, etc.). In some embodiments, the RPS27A 3′UTR sequence is a fragment of the endogenous (wild-type) RPS27A gene sequence. In some embodiments, the RPS27A 3′UTR sequence is a modified version of the RPS27A gene sequence (for example, comprises nucleotide changes, insertions, deletions, etc.).

In some embodiments, the engineered mRNAs comprise a modified 5′ terminal oligopyrimidine tract (TOP) removed. In some embodiments, the engineered mRNAs comprise a modification of one or more upstream translation start codons.

In some embodiments, the engineered mRNAs comprise a sequence for endoplasmic reticulum (ER) targeting of the target protein. In some embodiments, the engineered mRNAs comprise a calnexin sequence (for example, as disclosed in SEQ ID NOs:27 and 28).

In some embodiments, the engineered mRNAs comprise a sequence for mitochondria targeting of the target protein. In some embodiments, the engineered mRNAs comprise a TOM20 sequence (for example, as disclosed in SEQ ID NOs:29 and 30).

In some embodiments, the engineered mRNAs comprise a sequence for lysosome targeting of the target protein. In some embodiments, the engineered mRNAs comprise a CatB sequence (for example, as disclosed in SEQ ID NOs:31 and 32).

In some embodiments, the engineered mRNAs comprise a sequence for targeting of the of the target protein to the nucleus. In some embodiments, the engineered mRNAs comprise a nuclear localization signal sequence (NLS) sequence (for example, as disclosed in SEQ ID NOs:33 and 40).

In some aspects, disclosed herein is an engineered mRNA comprising: a first nucleic acid sequence comprising an engineered 5′ untranslated region (5′UTR) sequence; a second nucleic acid sequence comprising a heterologous nucleic acid sequence; and a third nucleic acid sequence comprising an RPS27A 3′ untranslated region (3′UTR) sequence.

In some embodiments, the engineered 5′UTR sequence is selected from the group comprising SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 81, SEQ ID NO: 82, SEQ ID NO: 83, SEQ ID NO: 84, SEQ ID NO: 85, or SEQ ID NO: 86.

In some embodiments, the engineered 5′UTR sequence is SEQ ID NO: 12. In some embodiments, the engineered 5′UTR sequence is SEQ ID NO: 13. In some embodiments, the engineered 5′UTR sequence is SEQ ID NO: 14. In some embodiments, the engineered 5′UTR sequence is SEQ ID NO: 15. In some embodiments, the engineered 5′UTR sequence is SEQ ID NO: 16. In some embodiments, the engineered 5′UTR sequence is SEQ ID NO: 17. In some embodiments, the engineered 5′UTR sequence is SEQ ID NO: 18. In some embodiments, the engineered 5′UTR sequence is SEQ ID NO: 19. In some embodiments, the engineered 5′UTR sequence is SEQ ID NO: 20. In some embodiments, the engineered 5′UTR sequence is SEQ ID NO: 21. In some embodiments, the engineered 5′UTR sequence is SEQ ID NO: 22. In some embodiments, the engineered 5′UTR sequence is SEQ ID NO: 23.

In some embodiments, the engineered 5′UTR sequence is SEQ ID NO: 81. In some embodiments, the engineered 5′UTR sequence is SEQ ID NO: 82. In some embodiments, the engineered 5′UTR sequence is SEQ ID NO: 83. In some embodiments, the engineered 5′UTR sequence is SEQ ID NO: 84. In some embodiments, the engineered 5′UTR sequence is SEQ ID NO: 85. In some embodiments, the engineered 5′UTR sequence is SEQ ID NO: 86.

In some embodiments, the engineered 5′UTR sequence is selected from the group comprising SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 81, SEQ ID NO: 82, SEQ ID NO: 83, SEQ ID NO: 84, SEQ ID NO: 85, or SEQ ID NO: 86, or a fragment or functionally active variant thereof.

In some embodiments, the engineered 5′UTR sequence is selected from the group comprising a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 81, SEQ ID NO: 82, SEQ ID NO: 83, SEQ ID NO: 84, SEQ ID NO: 85, or SEQ ID NO: 86.

In some embodiments, the engineered 5′UTR sequence comprises SEQ ID NO: 18 and the RPS27A 3′UTR sequence comprises SEQ ID NO: 24. In some embodiments, the engineered 5′UTR sequence comprises SEQ ID NO: 21 and the RPS27A 3′UTR sequence comprises SEQ ID NO: 24. In some embodiments, the engineered 5′UTR sequence comprises SEQ ID NO: 22 and the RPS27A 3′UTR sequence comprises SEQ ID NO: 24. In some embodiments, the engineered 5′UTR sequence comprises SEQ ID NO: 23 and the RPS27A 3′UTR sequence comprises SEQ ID NO: 24. In some embodiments, the engineered 5′UTR sequence comprises SEQ ID NO: 84 and the RPS27A 3′UTR sequence comprises SEQ ID NO: 24. In some embodiments, the engineered 5′UTR sequence comprises SEQ ID NO: 84 and the RPS27A 3′UTR sequence comprises SEQ ID NO: 87. In some embodiments, the engineered 5′UTR sequence comprises SEQ ID NO: 82 and the RPS27A 3′UTR sequence comprises SEQ ID NO: 24. In some embodiments, the engineered 5′UTR sequence comprises SEQ ID NO: 83 and the RPS27A 3′UTR sequence comprises SEQ ID NO: 24. In some embodiments, the engineered 5′UTR sequence comprises SEQ ID NO: 84 and the RPS27A 3′UTR sequence comprises SEQ ID NO: 89. In some embodiments, the engineered 5′UTR sequence comprises SEQ ID NO: 84 and the RPS27A 3′UTR sequence comprises SEQ ID NO: 91.

In some embodiments, the expression of the target protein is increased greater than about 10%, greater than about 20%, greater than about 30%, greater than about 40%, greater than about 50%, greater than about 60%, greater than about 70%, greater than about 80%, greater than about 90%, greater than about 100%, and more) when operably linked to the RPS27A 5′UTR sequence and/or the RPS27A 3′UTR sequence, in comparison to a control (for example, compared to the target protein's endogenous 5′UTR and/or 3′UTR, or compared to additional 5′UTR and/or 3′UTR sequences known in the art).

In some embodiments, the expression of the target protein is increased greater than about 10%, greater than about 20%, greater than about 30%, greater than about 40%, greater than about 50%, greater than about 60%, greater than about 70%, greater than about 80%, greater than about 90%, greater than about 100%, and more) when operably linked to the engineered 5′UTR sequence and/or the RPS27A 3′UTR sequence, in comparison to a control (for example, compared to the target protein's endogenous 5′UTR and/or 3′UTR, or compared to additional 5′UTR and/or 3′UTR sequences known in the art).

In some aspects, disclosed herein is a vector comprising the engineered mRNA of any preceding aspect. In some embodiments, a cell comprises the vector of any preceding aspect. In some embodiments, the cell is from the group comprising a mouse, a rat, a human, or a non-human primate. In some embodiments, the cell is from a mouse. In some embodiments, the cell is from a rat. In some embodiments, the cell is from a human. In some embodiments, the cell is from a non-human primate.

In some aspects, disclosed herein is a method of increasing protein expression, comprising the steps: introducing into a cell an engineered mRNA, comprising: a first nucleic acid sequence comprising an RPS27A 5′UTR sequence; a second nucleic acid sequence comprising a heterologous nucleic acid sequence; and a third nucleic acid sequence comprising an RPS27A 3′UTR sequence.

In some embodiments, the RPS27A 5′UTR sequence is selected from the group comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11. In some embodiments, the RPS27A 5′UTR sequence is SEQ ID NO: 1. In some embodiments, the RPS27A 5′UTR sequence is SEQ ID NO: 2. In some embodiments, the RPS27A 5′UTR sequence is SEQ ID NO: 3. In some embodiments, the RPS27A 5′UTR sequence is SEQ ID NO: 4. In some embodiments, the RPS27A 5′UTR sequence is SEQ ID NO: 5. In some embodiments, the RPS27A 5′UTR sequence is SEQ ID NO: 6. In some embodiments, the RPS27A 5′UTR sequence is SEQ ID NO: 7. In some embodiments, the RPS27A 5′UTR sequence is SEQ ID NO: 8. In some embodiments, the RPS27A 5′UTR sequence is SEQ ID NO: 9. In some embodiments, the RPS27A 5′UTR sequence is SEQ ID NO: 10. In some embodiments, the RPS27A 5′UTR sequence is SEQ ID NO: 11.

In some aspects, disclosed herein is a method of increasing protein expression, comprising the steps: introducing into a cell an engineered mRNA, comprising: a first nucleic acid sequence comprising an engineered 5′UTR sequence; a second nucleic acid sequence comprising a heterologous nucleic acid sequence; and a third nucleic acid sequence comprising an RPS27A 3′UTR sequence.

In some embodiments, the engineered 5′UTR sequence is selected from the group comprising SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 23. In some embodiments, the engineered 5′UTR sequence is SEQ ID NO: 12. In some embodiments, the engineered 5′UTR sequence is SEQ ID NO: 13. In some embodiments, the engineered 5′UTR sequence is SEQ ID NO: 14. In some embodiments, the engineered 5′UTR sequence is SEQ ID NO: 15. In some embodiments, the engineered 5′UTR sequence is SEQ ID NO: 16. In some embodiments, the engineered 5′UTR sequence is SEQ ID NO: 17. In some embodiments, the engineered 5′UTR sequence is SEQ ID NO: 18. In some embodiments, the engineered 5′UTR sequence is SEQ ID NO: 19. In some embodiments, the engineered 5′UTR sequence is SEQ ID NO: 20. In some embodiments, the engineered 5′UTR sequence is SEQ ID NO: 21. In some embodiments, the engineered 5′UTR sequence is SEQ ID NO: 22. In some embodiments, the engineered 5′UTR sequence is SEQ ID NO: 23.

In some embodiments, the nucleic acid sequences disclosed herein are isolated. In some embodiments, the nucleic acid sequences disclosed herein are recombinant.

In some embodiments, the heterologous nucleic acid sequence encodes a target protein. The heterologous nucleic acid sequence or target protein can be any nucleic acid sequence/protein of interest.

In some embodiments, the target protein comprises a fluorescent protein. In some embodiments, the target protein is fused to a fluorescent protein. In one embodiment, the fluorescent protein comprises mCherry (mCh). In some embodiments, the fluorescent protein comprises GFP. In some embodiments, the fluorescent protein comprises YFP.

In some embodiments, the RPS27A 3′UTR sequence is selected from the group comprising SEQ ID NO: 24, SEQ ID NO: 25, or SEQ ID NO: 26. In some embodiments, the RPS27A 3′UTR sequence is SEQ ID NO: 24. In some embodiments, the RPS27A 3′UTR sequence is SEQ ID NO: 25. In some embodiments, the RPS27A 3′UTR sequence is SEQ ID NO: 26.

In some aspects, disclosed herein is an engineered mRNA comprising: a first nucleic acid sequence comprising an RPS27A 5′UTR sequence; and a second nucleic acid sequence comprising a heterologous nucleic acid sequence. In some aspects, disclosed herein is an engineered mRNA comprising: a first nucleic acid sequence comprising an engineered 5′UTR sequence; and a second nucleic acid sequence comprising a heterologous nucleic acid sequence. In some aspects, disclosed herein is an engineered mRNA comprising: a nucleic acid sequence comprising an RPS27A 3′UTR sequence; and a second nucleic acid sequence comprising a heterologous nucleic acid sequence. These engineered mRNAs can be used in any of the vectors, cells, or methods described herein.

In the embodiments herein, the RPS27A 5′UTR sequence is operably linked to the heterologous nucleic acid sequence. In the embodiments herein, the engineered 5′UTR sequence is operably linked to the heterologous nucleic acid sequence. In the embodiments herein, the RPS27A 3′UTR sequence is operably linked to the heterologous nucleic acid sequence.

In some embodiments, the nucleic acids (engineered mRNAs) disclosed herein comprise at least one chemically modified nucleotide. In some embodiments, the at least one chemically modified nucleotide comprises a chemically modified nucleobase, a chemically modified ribose, a chemically modified phosphodiester linkage, or a combination thereof.

In one embodiment, the at least one chemically modified nucleotide is a chemically modified nucleobase.

In one embodiment, the chemically modified nucleobase is selected from 5-formylcytidine (5fC), 5-methylcytidine (5meC), 5-methoxycytidine (5moC), 5-hydroxycytidine (5hoC), 5-hydroxymethylcytidine (5hmC), 5-formyluridine (5fU), 5-methyluridine (5-meU), 5-methoxyuridine (5moU), 5-carboxymethylesteruridine (5camU), pseudouridine (Ψ), N¹-methylpseudouridine (me¹Ψ), N⁶-methyladenosine (me⁶A), or thienoguanosine (^{th}G).

In some embodiments, the chemically modified nucleobase is 5-methoxyuridine (5moU). In some embodiments, the chemically modified nucleobase is pseudouridine (Ψ). In some embodiments, the chemically modified nucleobase is N¹-methylpseudouridine (me¹Ψ).

The structures of these modified nucleobases are shown below:

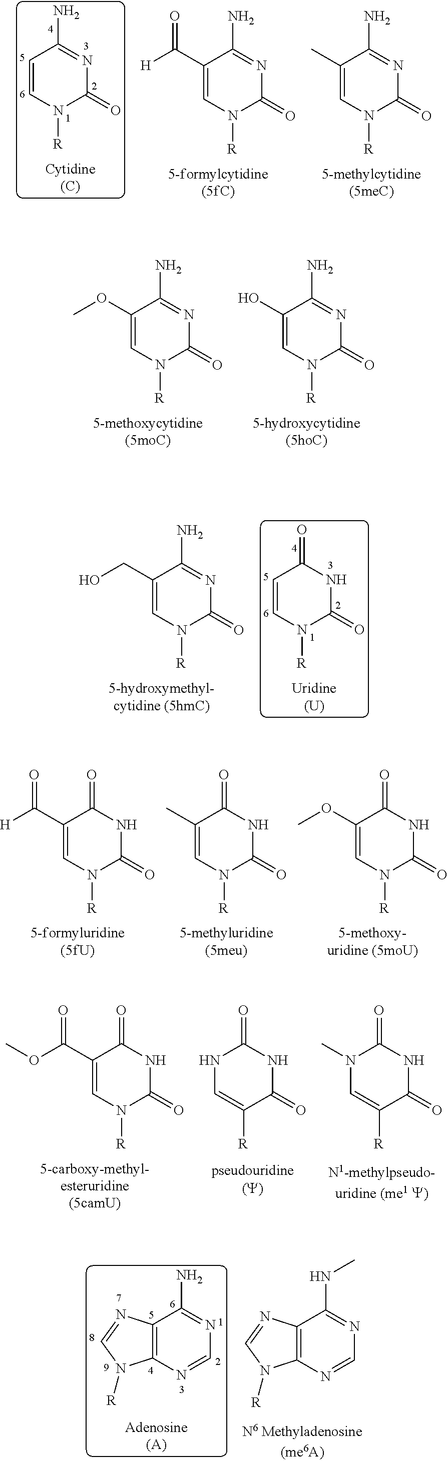

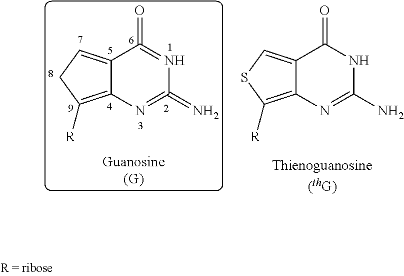

In one embodiment, the at least one chemically modified nucleotide is a chemically modified ribose.

In one embodiment, the chemically modified ribose is selected from 2′-O-methyl (2′-O-Me), 2′-Fluoro (2′-F), 2′-deoxy-2′-fluoro-beta-D-arabino-nucleic acid (2′F-ANA), 4′-S, 4′-SFANA, 2′-azido, UNA, 2′-O-methoxy-ethyl (2′-O-ME), 2′-O-Allyl, 2′-O-Ethylamine, 2′-O-Cyanoethyl, Locked nucleic acid (LAN), Methylene-cLAN, N-MeO-amino BNA, or N-MeO-aminooxy BNA. In one embodiment, the chemically modified ribose is 2′-G-methyl (2′-G-Me). In one embodiment, the chemically modified ribose is 2′-Fluoro (2′-F).

The structures of these modified riboses are shown below:

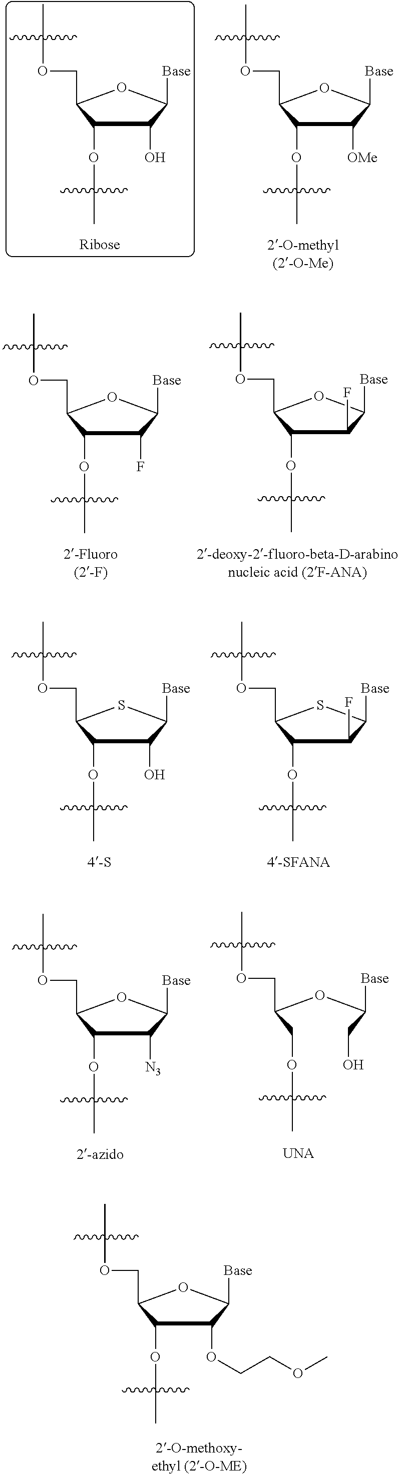

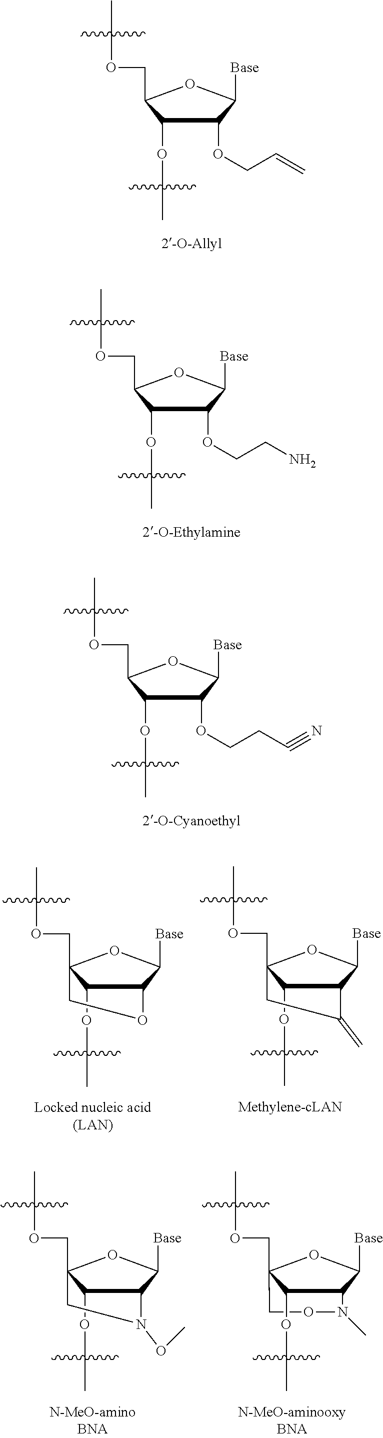

In one embodiment, the at least one chemically modified nucleotide is a chemically modified phosphodiester linkage.

In one embodiment, the chemically modified phosphodiester linkage is selected from phosphorothioate (PS), boranophosphate, phosphodithioate (PS2), 3′,5′-amide, N3′-phosphoramidate (NP), Phosphodiester (PO), or 2′,5′-phosphodiester (2′,5′-PO). In one embodiment, the chemically modified phosphodiester linkage is phosphorothioate.

The structures of these modified phosphodiester linkages are shown below:

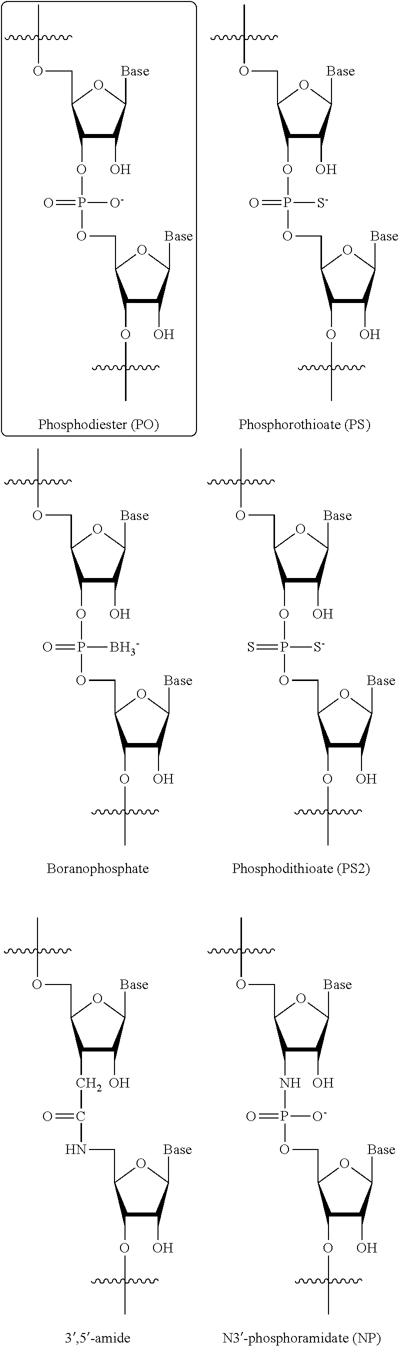

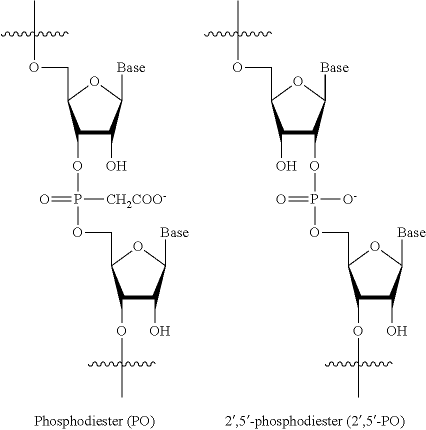

In some embodiments, the heterologous nucleic acid sequence is heterologous with respect to the 5′ UTR sequence. In some embodiments, the heterologous nucleic acid sequence is heterologous with respect to the 3′ UTR sequence. In some embodiments, the heterologous nucleic acid sequence is heterologous with respect to both the 5′ UTR sequence and the 3′ UTR sequence. In some aspects, disclosed herein is a vector comprising a nucleic acid encoding the engineered RNA of any preceding aspect. In some embodiments, the vector comprises the nucleic acid sequence selected from the group comprising SEQ ID NOs: 41 to 66.

In some aspects, disclosed herein is a cell comprising the engineered RNA or the vector of any preceding aspect.

In some aspects, disclosed herein in a method of increasing protein expression, comprising the steps:

introducing into a cell an engineered mRNA, comprising:

a first nucleic acid sequence comprising an RPS27A 5′UTR sequence or an engineered 5′ untranslated region (5′UTR) sequence;

a second nucleic acid sequence comprising a heterologous nucleic acid sequence; and

a third nucleic acid sequence comprising an RPS27A 3′UTR sequence.

In some aspects, disclosed herein is a vaccine for treating, preventing, reducing, and/or inhibiting a viral infection, said vaccine comprising an engineered mRNA comprising:

a first nucleic acid sequence comprising an RPS27A 5′ untranslated region (5′UTR) sequence or an engineered 5′ untranslated region (5′UTR) sequence;

a second nucleic acid sequence comprising a heterologous nucleic acid sequence; and

a third nucleic acid sequence comprising an RPS27A 3′ untranslated region (3′UTR) sequence, wherein the heterologous nucleic acid sequence encodes a viral protein.

In some embodiments, the viral protein is a COVID-19 protein, including, for example, COVID-19 spike protein, COVID-19 envelope protein, COVID-19 membrane protein, or COVID-19 nucleocapsid protein, or a fragment thereof. In some embodiments, the viral protein is a receptor binding domain of COVID-19 spike protein.

Accordingly, in some embodiments, the vaccine of any preceding aspect comprises an RNA sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 93, SEQ ID NO: 94, SEQ ID NO: 95, SEQ ID NO: 96, or SEQ ID NO: 97, or a functional fragment thereof. In some embodiments, the vaccine of any preceding aspect comprises an RNA sequence selected from the group comprising SEQ ID NO: 93, SEQ ID NO: 94, SEQ ID NO: 95, SEQ ID NO: 96, or SEQ ID NO: 97.

In some embodiments, the vaccine further comprises an adjuvant. In some embodiments, the vaccine further comprises a pharmaceutically acceptable carrier.

In some aspects, disclosed herein is a method of treating, preventing, reducing, and/or inhibiting a viral infection in a subject, comprising administering to the subject an effective amount of the vaccine of any preceding aspect.

### EXAMPLES

The following examples are set forth below to illustrate the compounds, systems, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1

Luciferase mRNAs with modified 5′ UTR and 3′ UTR from mouse ribosomal protein S27a gene outperformed those mRNAs with UTRs published in literature in A549 and Hep3B cells. AG, AG+G, AG+G w/o 3UTR and CYBA are control luciferase mRNAs with identical coding sequences as other engineered mRNAs. 5′ UTR and 3′ UTR of AG are from Human Alpha Globin gene (Gene symbol: HBA1). AG+G is modified AG with one extra G inserted at the end of 5′ UTR to create a complete Kozak sequence (GCCACC). AG+G w/o 3UTR had the same 5′ UTR as AG+G and 3′ UTR removed. CYBA had 5′UTR and 3′UTR from human cytochrome b-245 alpha polypeptide gene (Gene symbol: CYBA). All mRNAs were delivered by lipofectamine 3000.

### Example 2

The eGFP mRNAs with unnatural 5′ UTR further enhanced protein expression in A549, Hep3B and 293T cells (n=2). AG+G w/o 3UTR and CYBA are control luciferase mRNAs as described in Example 1. All mRNAs were delivered by lipofectamine 3000.

### Example 3

The luciferase mRNA with 5UTR-18 and 3UTR-1 showed increased protein expression with pseudouridine modification (pU) than unmodified mRNA in A549 cells (n=3). All mRNAs were delivered by lipofectamine 3000.

### Example 4

The pseudouridine modified luciferase mRNA with 5UTR-22+3UTR-1 and 5UTR-23+3UTR-1 showed selective gene expression in a liver tumor cell line (Hep3B) compared to that in a lung tumor cell line (A549). All mRNAs were delivered by lipofectamine 3000 (n=3).

### Example 5

The organelle targeting eGFP/mCherry mRNAs with 5′ UTR and 3′ UTR sequence disclosed here can be applied for organelle imaging in live Hep3B cells. The organelle imaging capability of these organelles targeting eGFP/mCherry mRNAs were verified by colocalization with commercially available organelle imaging probes. All mRNAs were delivered by lipofectamine 3000.

### Example 6

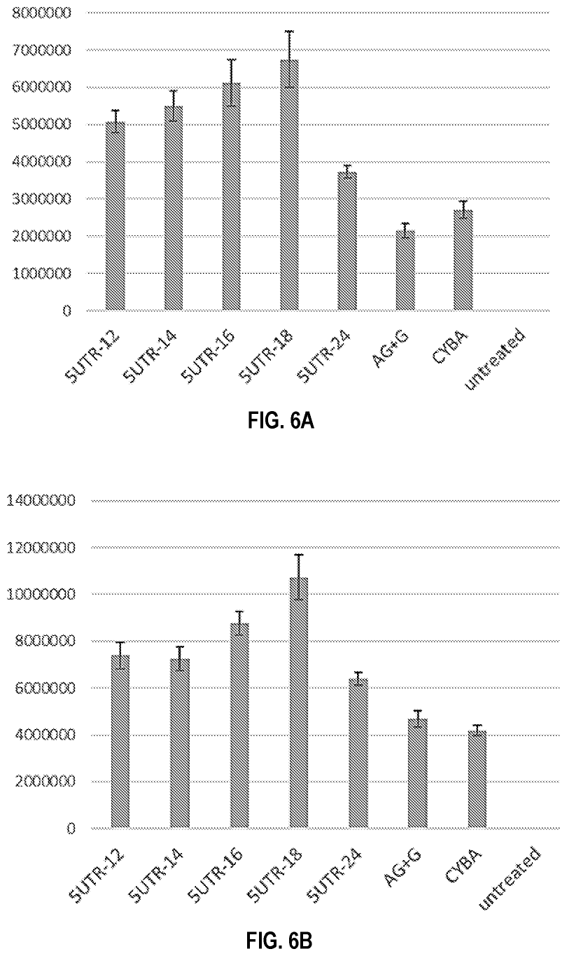

The results in FIG. 6A and FIG. 6B were obtained in Hep3B and 293T cells, respectively. All mRNAs utilized the same 3′ UTR: 3UTR1. All mRNAs were synthesized using pseudouridine to fully replace UTPs in in vitro transcription. The mRNA with 5′ UTR of 70 nt showed the highest expression. AG+G and CYBA are control luciferase mRNAs with previously published UTRs. 5′ UTR and 3′ UTR of AG+G are from Human Alpha Globin gene (Gene symbol: HBA1) with one extra G inserted at the end of 5′ UTR to create a complete Kozak sequence (GCCACC). CYBA had 5′UTR and 3′UTR from human cytochrome b-245 alpha polypeptide gene (Gene symbol: CYBA). All mRNAs were delivered by lipofectamine 3000.

### Example 7

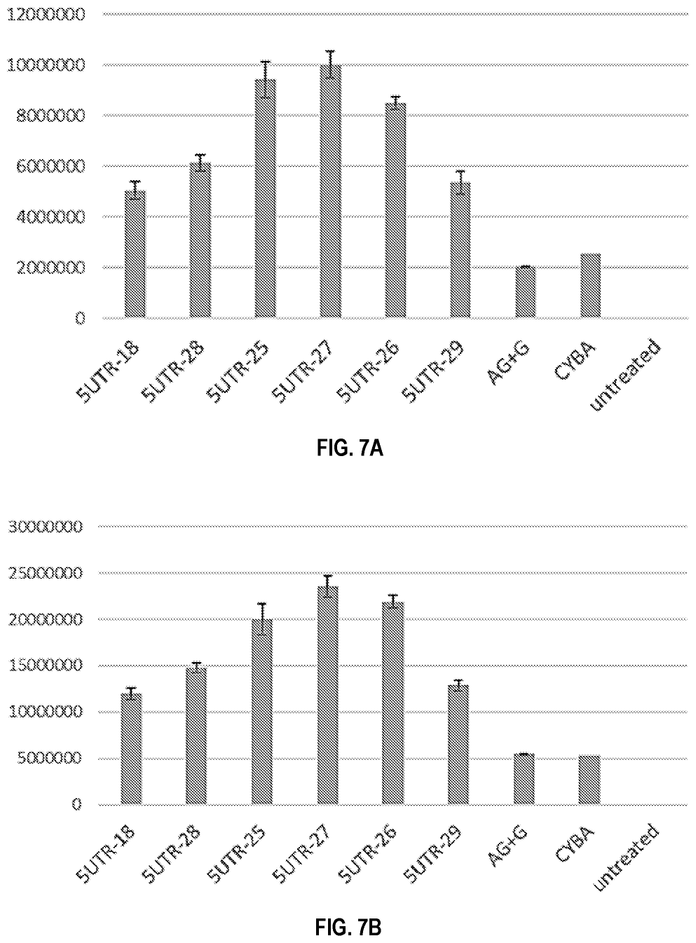

The results in FIG. 7A and FIG. 7B were obtained in Hep3B and 293T cells, respectively. All mRNAs utilized the same 3′ UTR: 3UTR-1. All mRNAs were synthesized using pseudouridine to fully replace UTPs in in vitro transcription. The removal of microRNA target sites in 5UTR-18 generated 5UTR-28. The removal of microRNA target sites in 5UTR-25 generated 5UTR-27. The removal of microRNA target sites in 5UTR-26 generated 5UTR-29. The mRNA with 5UTR-27 showed the highest expression. AG+G and CYBA are control luciferase mRNAs with previously published UTRs. 5′ UTR and 3′ UTR of AG+G are from Human Alpha Globin gene (Gene symbol: HBA1) with one extra G inserted at the end of 5′ UTR to create a complete Kozak sequence (GCCACC). CYBA had 5′UTR and 3′UTR from human cytochrome b-245 alpha polypeptide gene (Gene symbol: CYBA). All mRNAs were delivered by lipofectamine 3000.

### Example 8

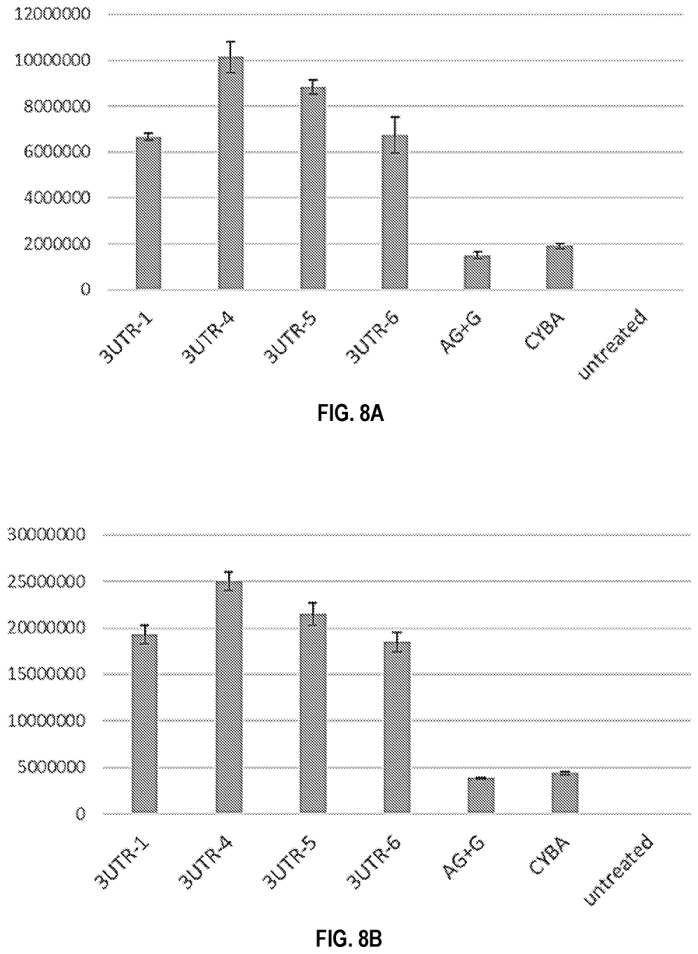

The results in FIG. 8A and FIG. 8B were obtained in Hep3B and 293T cells, respectively. All mRNAs utilized the same 5′ UTR: 5UTR-27. Addition of a functional motif A to 3UTR-1 generated 3UTR-4. Addition of a functional motif B to 3UTR-1 generated 3UTR-5. Addition of a functional motif C to 3UTR-1 generated 3UTR-6. The mRNA with 3UTR-4 showed the highest expression. All mRNAs were synthesized using pseudouridine to fully replace UTPs in in vitro transcription. AG+G and CYBA are control luciferase mRNAs with previously published UTRs. 5′ UTR and 3′ UTR of AG+G are from Human Alpha Globin gene (Gene symbol: HBA1) with one extra G inserted at the end of 5′ UTR to create a complete Kozak sequence (GCCACC). CYBA had 5′UTR and 3′UTR from human cytochrome b-245 alpha polypeptide gene (Gene symbol: CYBA). All mRNAs were delivered by lipofectamine 3000.

SEQUENCES
5UTR-1 (T44)
5′ UTR from transcript ENSMUST00000102844 of mouse ribosomal
protein S27a gene (Gene symbol: RPS27A)
(SEQ ID NO: 1)
GGGUUUCCGAUCCGCCAUCGUGGGUGAGUGUAUGCUCUGUGGCCGCGCUCUGG
CUAGUGGCGCUACGCGUCGCUCUCACGGGUGUCGUCGGAUCUAAUCCGUCUCU
UUUCGAAUGCAGGUGGAGCCGCCGCCACG
5UTR-2 (T44-top)
Modification of 5UTR-1 with 5′ terminal oligopyrimidine tract
(5′ TOP) removed
(SEQ ID NO: 2)
GGGGAUCCGCCAUCGUGGGUGAGUGUAUGCUCUGUGGCCGCGCUCUGGCUAGU
GGCGCUACGCGUCGCUCUCACGGGUGUCGUCGGAUCUAAUCCGUCUCUUUUCG
AAUGCAGGUGGAGCCGCCGCCACG
5UTR-3 (T44-top-uAUG)
Modification of 5UTR-2: two upstream translation start codons AUG
modified to UAG
(SEQ ID NO: 3)
GGGGAUCCGCCAUCGUGGGUGAGUGUUAGCUCUGUGGCCGCGCUCUGGCUAGU
GGCGCUACGCGUCGCUCUCACGGGUGUCGUCGGAUCUAAUCCGUCUCUUUUCG
AUAGCAGGUGGAGCCGCCGCCACG
5UTR-4 (Truncated-T44-top-uAUG)
Modification of 5UTR-3 with the first 83 nucleotides after GGG
truncated
(SEQ ID NO: 4)
GGGAUCUAAUCCGUCUCUUUUCGAUAGCAGGUGGAGCCGCCGCCACG
5UTR-5 (Truncated-T44-top-uAUG-2AUG)
Modification of 5UTR-4 with one additional AUG added before
the AUG in coding region, resulting two tandem AUG translation
start codons
(SEQ ID NO: 5)
GGGAUCUAAUCCGUCUCUUUUCGAUAGCAGGUGGAGCCGCCGCCACGAUG
5UTR-6 (T45)
5′ UTR from transcript ENSMUST00000102845 of mouse ribosomal protein
S27a gene (Gene symbol: RPS27A)
(SEQ ID NO: 6)
GGGAGGAAAGCCUCUCUUAAUCGCAUCGGCUGUAUAAGAAAGCCUUUUGAGG
CAUUUUUUUUAGUUGAGCACAUCAUUUCGAGGCCAUUCUGAGGUAAACCGAG
AAAAGAGCGUAAAGAAACCGAGCGAACGAGCAAAUCUGGCACUGCGUUAGAC
AGCCGCGAUUCCGCUGCAGCGCGCAGGCACGUGUGUGGCCGCCUAAGGGGCGG
GUCCUUCGGCCAGGAGACCCCGUCGGCCACGCUCGGAUCUUCCUUUCCGAUCC
GCCAUCGUGGGUGGAGCCGCCGCCACG
5UTR-7 (T45-top)
Modification of 5UTR-6 with 5′ terminal oligopyrimidine tract
(5′ TOP) removed
(SEQ ID NO: 7)
GGGAGGAAAGAAUCGCAUCGGCUGUAUAAGAAAGCCUUUUGAGGCAUUUUUU
UUAGUUGAGCACAUCAUUUCGAGGCCAUUCUGAGGUAAACCGAGAAAAGAGC
GUAAAGAAACCGAGCGAACGAGCAAAUCUGGCACUGCGUUAGACAGCCGCGAU
UCCGCUGCAGCGCGCAGGCACGUGUGUGGCCGCCUAAGGGGCGGGUCCUUCGG
CCAGGAGACCCCGUCGGCCACGCUCGGAUCUUCCUUUCCGAUCCGCCAUCGUG
GGUGGAGCCGCCGCCACG
5UTR-8 (T17)
5′UTR from transcript ENST00000272317 of human ribosomal protein
S27a gene (Gene symbol: RPS27A)
(SEQ ID NO: 8)
GGGCCCCUCGACCUCCUUUUAAAAAUUCUCUUAGCCACGUUGAUUGUACGGGA
AAAGCCUUUUUAAAACAUCUUUUACGUUGCUUAAACCUACAGUUUCGAAAGC
AUUCCGAAGGCUAAAGUGAGAAAUAAGCCCAGGCUAGGGAGAGGAGAAACGA
AGUUCACGUCCUAGUCUGGCACCGGGUUGGAUUGUCGCUGGGACGGCAGUCAG
GCAUUUGGUGUGGUCGCCUAAGGGGUGGGUCCUUCGGCGGGAGCUCCGGGAA
ACCCCGUGGGCCUGCGCGGCGUUCUUCCUUUUCGAUCCGCCAUCUGCGGUGGA
GCCGCCACCAAA
5UTR-9 (T17-TOP)
Modification of 5UTR-8 with 5′ terminal oligopyrimidine
tract (5′ TOP) removed
(SEQ ID NO: 9)
GGGAGCCACGUUGAUUGUACGGGAAAAGCCUUUUUAAAACAUCUUUUACGUU
GCUUAAACCUACAGUUUCGAAAGCAUUCCGAAGGCUAAAGUGAGAAAUAAGC
CCAGGCUAGGGAGAGGAGAAACGAAGUUCACGUCCUAGUCUGGCACCGGGUU
GGAUUGUCGCUGGGACGGCAGUCAGGCAUUUGGUGUGGUCGCCUAAGGGGUG
GGUCCUUCGGCGGGAGCUCCGGGAAACCCCGUGGGCCUGCGCGGCGUUCUUCC
UUUUCGAUCCGCCAUCUGCGGUGGAGCCGCCACCAAA
5UTR-10 (T35)
5′UTR from transcript ENST00000404735 of human ribosomal
protein S27a gene (Gene symbol: RPS27A)
(SEQ ID NO: 10)
GGGCGUUCUUCCUUUUCGAUCCGCCAUCUGCGGUGGGUGUCUGCACUUCGGCU
GCUCUCGGGUUAGCACCCUAUGGUGCCUUCUCUUGUGAUCCCUGACCUAACCU
GUCUCUUCCUUUUCCUCAACCUCAGGUGGAGCCGCCACCAAA
5UTR-11 (T35-TOP)
Modification of 5UTR-10 with 5′ terminal oligopyrimidine
tract (5′ TOP) removed
(SEQ ID NO: 11)
GGGCGCGAUCCGCCAUCUGCGGUGGGUGUCUGCACUUCGGCUGCUCUCGGGUU
AGCACCCUAUGGUGCCUUCUCUUGUGAUCCCUGACCUAACCUGUCUCUUCCUU
UUCCUCAACCUCAGGUGGAGCCGCCACCAAA
5UTR-12 (10nt)
10nt unnatural 5′ UTR with GGG, kozak sequence (GCCACC) and
minimal secondary structure
(SEQ ID NO: 12)
GGGAGCCACC
5UTR-13 (20nt)
20nt unnatural 5′ UTR with GGG, kozak sequence (GCCACC) and
minimal secondary structure
(SEQ ID NO: 13)
GGGGACAGAAAACAGCCACC
5UTR-14 (30nt)
30nt unnatural 5′ UTR with GGG, kozak sequence (GCCACC) and
minimal secondary structure
(SEQ ID NO: 14)
GGGAAAGAAACAGGACAGAAAACAGCCACC
5UTR-15 (40nt)
40nt unnatural 5′ UTR with GGG, kozak sequence (GCCACC) and
minimal secondary structure
(SEQ ID NO: 15)
GGGAACACAUACAAAAGAAACAGGACAGAAAACAGCCACC
5UTR-16 (50nt)
50nt unnatural 5′ UTR with GGG, kozak sequence (GCCACC) and
minimal secondary structure
(SEQ ID NO: 16)
GGGAACGACAAGAAACACAUACAAAAGAAACAGGACAGAAAACAGCCACC
5UTR-17 (60nt)
60nt unnatural 5′ UTR with GGG, kozak sequence (GCCACC) and
minimal secondary structure
(SEQ ID NO: 17)
GGGCAUAAACAUAAACGACAAGAAACACAUACAAAAGAAACAGGACAGAAAA
CAGCCACC
5UTR-18 (70nt = 0305K)
70nt unnatural 5′ UTR with GGG, kozak sequence (GCCACC) and
minimal secondary structure
(SEQ ID NO: 18)
GGGAAGAGAUAAACAUAAACAUAAACGACAAGAAACACAUACAAAAGAAACA
GGACAGAAAACAGCCACC
5UTR-19 (100nt)
100nt unnatural 5′ UTR with GGG, kozak sequence (GCCACC) and
minimal secondary structure
(SEQ ID NO: 19)
GGGAACAACAGAGGAGAAGAGGGAACAGGACACAAGAGAUAAACAUAAACAU
AAACGACAAGAAACACAUACAAAAGAAACAGGACAGAAAACAGCCACC
5UTR-20 (50nt = 0301K-1)
Alternative 50nt unnatural 5′ UTR with GGG, kozak sequence (GCCACC)
and minimal secondary structure
(SEQ ID NO: 20)
GGGAAAGAAAAAGAUAAGGAGAAAAAUAAAGAGAGGAAGAAAAAGCCACC
5UTR-21 (50nt = 0301K-2)
Alternative 50nt unnatural 5′ UTR with GGG, kozak sequence (GCCACC)
and minimal secondary structure
(SEQ ID NO: 21)
GGGAAAAGUAGAAAGAAAGAAAGAAGAGAAAAUAAAGACAAAGAGCCACC
5UTR-22 (70nt = 1015K-A)
70nt unnatural 5′ UTR with G, kozak sequence (GCCACC), minimal
secondary structure and modified ACGU content (25% GC, 27% A, 37% U)
(SEQ ID NO: 22)
GCUUUCACUAUUUCAUUCAUUUCAUUCACACAUUACACUUACAUCACAUCCAC
AUUACAUUUCUGCCACC
5UTR-23 (70nt = 1015K-B)
70nt unnatural 5′ UTR with G, kozak sequence (GCCACC), minimal
secondary structure and modified ACGU content (25% GC, 17% A, 48% U)
(SEQ ID NO: 23)
GCUUUCACUAUUUCAUUCAUUUCAUUCUCUCAUUACUCUUACUUCUCUUCCUC
AUUACAUUUCUGCCACC
3UTR-1 (T44/45)
3′ UTR from transcript ENSMUST00000102844 and ENSMUST00000102845
of mouse ribosomal protein S27a gene (Gene symbol: RPS27A)
(SEQ ID NO: 24)
UUGUGUAUGCGUUAAUAAAAAGAAGGAACUCGUA
3UTR-2 (T35)
3′UTR from transcript ENST00000404735 of human ribosomal protein
S27a gene (Gene symbol: RPS27A)
(SEQ ID NO: 25)
CUGUAUGAGUUAAUAAAAGACAUGAACUAACAUUUAUUGUUGGGUUUUAUUG
CAGUAAAAAGAAUGGUUUUUAAGCACCAAAUUGAUGGUCACACCAUUUCCUU
UUAGUAGUGCUACUGCUAUCGCUGUGUGAAUGUUGCCUCUGGGGAUUAUGUG
ACCCAGUGGUUCUGUAUACCUG
3UTR-3 (T17)
3′UTR from transcript ENST00000272317 of human ribosomal
protein S27a gene (Gene symbol: RPS27A)
(SEQ ID NO: 26)
CUGUAUGAGUUAAUAAAAGACAUGAACUAACAUUUAUUGUUGGGUUUUAUUG
CAGUAAAAAGAAUGGUUUUUAAGCACCAAAUUGAUGGUCACACCAUUUCCUU
UUAGUAGUGCUACUGCUAUCGCUGUGUGAAUGUUGCCUCUGGGGAUUAUGUG
ACCCAGUGGUUCUGUAUACCUGCCAGGUGCCAACCACUUGUAAAGGUCUUGAU
AUUUUCAAUUCUUAGACUACCUAUACUUUGGCAGAAGUUAUAUUUAAUGUAA
GUUGUCUAAAUAUAA
T44-TOP-uAUG-Calnexin-EGFP (ER targeting eGFP mRNA)
(SEQ ID NO: 27)
GAUCCGCCAUCGUGGGUGAGUGUUAGCUCUGUGGCCGCGCUCUGGCUAGUGGC
GCUACGCGUCGCUCUCACGGGUGUCGUCGGAUCUAAUCCGUCUCUUUUCGAUA
GCAGGUGGAGCCGCCGCCACGAUGGAAGGGAAGUGGUUGCUGUGUAUGUUAC
UGGUGCUUGGAACUGCUAUUGUUGAGGCUCAUGAUGGACAUGAUGAUGAUGU
GAUUGAUAUUGAGGAUGACCUUGACGAUGUCAUUGAAGAGGUAGAAGACUCA
AAACCAGAUACCACUGCUCCUCCUUCAUCUCCCAAGGUUACUUACAAAGCUCC
AGUUCCAACAGGGGAAGUAUAUUUUGCUGAUUCUUUUGACAGAGGAACUCUG
UCAGGGUGGAUUUUAUCCAAAGCCAAGAAAGACGAUACCGAUGAUGAAAUUG
CCAAAUAUGAUGGAAAGUGGGAGGUAGAGGAAAUGAAGGAGUCAAAGCUUCC
AGGUGAUAAAGGACUUGUGUUGAUGUCUCGGGCCAAGCAUCAUGCCAUCUCU
GCUAAACUGAACAAGCCCUUCCUGUUUGACACCAAGCCUCUCAUUGUUCAGUA
UGAGGUUAAUUUCCAAAAUGGAAUAGAAUGUGGUGGUGCCUAUGUGAAACUG
CUUUCUAAAACACCAGAACUCAACCUGGAUCAGUUCCAUGACAAGACCCCUUA
UACGAUUAUGUUUGGUCCAGAUAAAUGUGGAGAGGACUAUAAACUGCACUUC
AUCUUCCGACACAAAAACCCCAAAACGGGUAUCUAUGAAGAAAAACAUGCUAA
GAGGCCAGAUGCAGAUCUGAAGACCUAUUUUACUGAUAAGAAAACACAUCUU
UACACACUAAUCUUGAAUCCAGAUAAUAGUUUUGAAAUACUGGUUGACCAAU
CUGUGGUGAAUAGUGGAAAUCUGCUCAAUGACAUGACUCCUCCUGUAAAUCC
UUCACGUGAAAUUGAGGACCCAGAAGACCGGAAGCCCGAGGAUUGGGAUGAA
AGACCAAAAAUCCCAGAUCCAGAAGCUGUCAAGCCAGAUGACUGGGAUGAAG
AUGCCCCUGCUAAGAUUCCAGAUGAAGAGGCCACAAAACCCGAAGGCUGGUUA
GAUGAUGAGCCUGAGUACGUACCUGAUCCAGACGCAGAGAAACCUGAGGAUU
GGGAUGAAGACAUGGAUGGAGAAUGGGAGGCUCCUCAGAUUGCCAACCCUAG
AUGUGAGUCAGCUCCUGGAUGUGGUGUCUGGCAGCGACCUGUGAUUGACAAC
CCCAAUUAUAAAGGCAAAUGGAAGCCUCCUAUGAUUGACAAUCCCAGUUACCA
GGGAAUCUGGAAACCCAGGAAAAUACCAAAUCCAGAUUUCUUUGAAGAUCUG
GAACCUUUCAGAAUGACUCCUUUUAGUGCUAUUGGUUUGGAGCUGUGGUCCA
UGACCUCUGACAUUUUUUUUGACAACUUUAUCAUUUGUGCUGAUCGAAGAAU
AGUUGAUGAUUGGGCCAAUGAUGGAUGGGGCCUGAAGAAAGCUGCUGAUGGG
GCUGCUGAGCCAGGCGUUGUGGGGCAGAUGAACGAGGCAGCUGAAGAGCGCCC
GUGGCUGUGGGUAGUCUAUAUUCUAACUGUAGCCCUUCCUGUGUUCCUGGUU
AUCCUCUUCUGCUGUUCUGGAAAGAAACAGACCAGUGGUAUGGAGUAUAAGA
AAACUGAUGCACCUCAACCGGAUGUGAAGGAAGAGGAAGAAGAGAAGGAAGA
GGAAAAGGACAAGGGAGAUGAGGAGGAGGAAGGAGAAGAGAAACUUGAAGAG
AAACAGAAAAGUGAUGCUGAAGAAGAUGGUGGCACUGUCAGUCAAGAGGAGG
AAGACAGAAAACCUAAAGCAGAGGAGGAUGAAAUUUUGAACAGAUCACCAAG
AAACAGAAAGCCACGAAGAGAGCUCGAGGUGAGCAAGGGCGAGGAGCUGUUC
ACCGGGGUGGUGCCCAUCCUGGUCGAGCUGGACGGCGACGUAAACGGCCACAA
GUUCAGCGUGUCCGGCGAGGGCGAGGGCGAUGCCACCUACGGCAAGCUGACCC
UGAAGUUCAUCUGCACCACCGGCAAGCUGCCCGUGCCCUGGCCCACCCUCGUG
ACCACCCUGACCUACGGCGUGCAGUGCUUCAGCCGCUACCCCGACCACAUGAA
GCAGCACGACUUCUUCAAGUCCGCCAUGCCCGAAGGCUACGUCCAGGAGCGCA
CCAUCUUCUUCAAGGACGACGGCAACUACAAGACCCGCGCCGAGGUGAAGUUC
GAGGGCGACACCCUGGUGAACCGCAUCGAGCUGAAGGGCAUCGACUUCAAGGA
GGACGGCAACAUCCUGGGGCACAAGCUGGAGUACAACUACAACAGCCACAACG
UCUAUAUCAUGGCCGACAAGCAGAAGAACGGCAUCAAGGUGAACUUCAAGAU
CCGCCACAACAUCGAGGACGGCAGCGUGCAGCUCGCCGACCACUACCAGCAGA
ACACCCCCAUCGGCGACGGCCCCGUGCUGCUGCCCGACAACCACUACCUGAGC
ACCCAGUCCGCCCUGAGCAAAGACCCCAACGAGAAGCGCGAUCACAUGGUCCU
GCUGGAGUUCGUGACCGCCGCCGGGAUCACUCUCGGCAUGGACGAGCUGUACA
AGUCUAGAUGAUUGUGUAUGCGUUAAUAAAAAGAAGGAACUCGUA
T44-TOP-uAUG-Calnexin-mCherry (ER targeting mCherry mRNA)
(SEQ ID NO: 28)
GAUCCGCCAUCGUGGGUGAGUGUUAGCUCUGUGGCCGCGCUCUGGCUAGUGGC
GCUACGCGUCGCUCUCACGGGUGUCGUCGGAUCUAAUCCGUCUCUUUUCGAUA
GCAGGUGGAGCCGCCGCCACGAUGGAAGGGAAGUGGUUGCUGUGUAUGUUAC
UGGUGCUUGGAACUGCUAUUGUUGAGGCUCAUGAUGGACAUGAUGAUGAUGU
GAUUGAUAUUGAGGAUGACCUUGACGAUGUCAUUGAAGAGGUAGAAGACUCA
AAACCAGAUACCACUGCUCCUCCUUCAUCUCCCAAGGUUACUUACAAAGCUCC
AGUUCCAACAGGGGAAGUAUAUUUUGCUGAUUCUUUUGACAGAGGAACUCUG
UCAGGGUGGAUUUUAUCCAAAGCCAAGAAAGACGAUACCGAUGAUGAAAUUG
CCAAAUAUGAUGGAAAGUGGGAGGUAGAGGAAAUGAAGGAGUCAAAGCUUCC
AGGUGAUAAAGGACUUGUGUUGAUGUCUCGGGCCAAGCAUCAUGCCAUCUCU
GCUAAACUGAACAAGCCCUUCCUGUUUGACACCAAGCCUCUCAUUGUUCAGUA
UGAGGUUAAUUUCCAAAAUGGAAUAGAAUGUGGUGGUGCCUAUGUGAAACUG
CUUUCUAAAACACCAGAACUCAACCUGGAUCAGUUCCAUGACAAGACCCCUUA
UACGAUUAUGUUUGGUCCAGAUAAAUGUGGAGAGGACUAUAAACUGCACUUC
AUCUUCCGACACAAAAACCCCAAAACGGGUAUCUAUGAAGAAAAACAUGCUAA
GAGGCCAGAUGCAGAUCUGAAGACCUAUUUUACUGAUAAGAAAACACAUCUU
UACACACUAAUCUUGAAUCCAGAUAAUAGUUUUGAAAUACUGGUUGACCAAU
CUGUGGUGAAUAGUGGAAAUCUGCUCAAUGACAUGACUCCUCCUGUAAAUCC
UUCACGUGAAAUUGAGGACCCAGAAGACCGGAAGCCCGAGGAUUGGGAUGAA
AGACCAAAAAUCCCAGAUCCAGAAGCUGUCAAGCCAGAUGACUGGGAUGAAG
AUGCCCCUGCUAAGAUUCCAGAUGAAGAGGCCACAAAACCCGAAGGCUGGUUA
GAUGAUGAGCCUGAGUACGUACCUGAUCCAGACGCAGAGAAACCUGAGGAUU
GGGAUGAAGACAUGGAUGGAGAAUGGGAGGCUCCUCAGAUUGCCAACCCUAG
AUGUGAGUCAGCUCCUGGAUGUGGUGUCUGGCAGCGACCUGUGAUUGACAAC
CCCAAUUAUAAAGGCAAAUGGAAGCCUCCUAUGAUUGACAAUCCCAGUUACCA
GGGAAUCUGGAAACCCAGGAAAAUACCAAAUCCAGAUUUCUUUGAAGAUCUG
GAACCUUUCAGAAUGACUCCUUUUAGUGCUAUUGGUUUGGAGCUGUGGUCCA
UGACCUCUGACAUUUUUUUUGACAACUUUAUCAUUUGUGCUGAUCGAAGAAU
AGUUGAUGAUUGGGCCAAUGAUGGAUGGGGCCUGAAGAAAGCUGCUGAUGGG
GCUGCUGAGCCAGGCGUUGUGGGGCAGAUGAACGAGGCAGCUGAAGAGCGCCC
GUGGCUGUGGGUAGUCUAUAUUCUAACUGUAGCCCUUCCUGUGUUCCUGGUU
AUCCUCUUCUGCUGUUCUGGAAAGAAACAGACCAGUGGUAUGGAGUAUAAGA
AAACUGAUGCACCUCAACCGGAUGUGAAGGAAGAGGAAGAAGAGAAGGAAGA
GGAAAAGGACAAGGGAGAUGAGGAGGAGGAAGGAGAAGAGAAACUUGAAGAG
AAACAGAAAAGUGAUGCUGAAGAAGAUGGUGGCACUGUCAGUCAAGAGGAGG
AAGACAGAAAACCUAAAGCAGAGGAGGAUGAAAUUUUGAACAGAUCACCAAG
AAACAGAAAGCCACGAAGAGAGCUCGAGGUGAGCAAGGGCGAGGAGGAUAAC
AUGGCCAUCAUCAAGGAGUUCAUGCGCUUCAAGGUGCACAUGGAGGGCUCCGU
GAACGGCCACGAGUUCGAGAUCGAGGGCGAGGGCGAGGGCCGCCCCUACGAGG
GCACCCAGACCGCCAAGCUGAAGGUGACCAAGGGUGGCCCCCUGCCCUUCGCC
UGGGACAUCCUGUCCCCUCAGUUCAUGUACGGCUCCAAGGCCUACGUGAAGCA
CCCCGCCGACAUCCCCGACUACUUGAAGCUGUCCUUCCCCGAGGGCUUCAAGU
GGGAGCGCGUGAUGAACUUCGAGGACGGCGGCGUGGUGACCGUGACCCAGGAC
UCCUCCCUGCAGGACGGCGAGUUCAUCUACAAGGUGAAGCUGCGCGGCACCAA
CUUCCCCUCCGACGGCCCCGUAAUGCAGAAGAAGACCAUGGGCUGGGAGGCCU
CCUCCGAGCGGAUGUACCCCGAGGACGGCGCCCUGAAGGGCGAGAUCAAGCAG
AGGCUGAAGCUGAAGGACGGCGGCCACUACGACGCUGAGGUCAAGACCACCUA
CAAGGCCAAGAAGCCCGUGCAGCUGCCCGGCGCCUACAACGUCAACAUCAAGU
UGGACAUCACCUCCCACAACGAGGACUACACCAUCGUGGAACAGUACGAACGC
GCCGAGGGCCGCCACUCCACCGGCGGCAUGGACGAGCUGUACAAGUCUAGAUG
AUUGUGUAUGCGUUAAUAAAAAGAAGGAACUCGUA
T44-TOP-uAUG-TOM20-EGFP (Mitochondria targeting eGFP mRNA)
(SEQ ID NO: 29)
GAUCCGCCAUCGUGGGUGAGUGUUAGCUCUGUGGCCGCGCUCUGGCUAGUGGC
GCUACGCGUCGCUCUCACGGGUGUCGUCGGAUCUAAUCCGUCUCUUUUCGAUA
GCAGGUGGAGCCGCCGCCACGAUGGUGGGACGGAACAGCGCCAUCGCUGCAGG
AGUGUGCGGUGCCCUCUUCAUAGGGUACUGCAUCUACUUUGACCGCAAAAGGA
GGAGUGACCCCAACCUCGAGGUGAGCAAGGGCGAGGAGCUGUUCACCGGGGUG
GUGCCCAUCCUGGUCGAGCUGGACGGCGACGUAAACGGCCACAAGUUCAGCGU
GUCCGGCGAGGGCGAGGGCGAUGCCACCUACGGCAAGCUGACCCUGAAGUUCA
UCUGCACCACCGGCAAGCUGCCCGUGCCCUGGCCCACCCUCGUGACCACCCUGA
CCUACGGCGUGCAGUGCUUCAGCCGCUACCCCGACCACAUGAAGCAGCACGAC
UUCUUCAAGUCCGCCAUGCCCGAAGGCUACGUCCAGGAGCGCACCAUCUUCUU
CAAGGACGACGGCAACUACAAGACCCGCGCCGAGGUGAAGUUCGAGGGCGACA
CCCUGGUGAACCGCAUCGAGCUGAAGGGCAUCGACUUCAAGGAGGACGGCAAC
AUCCUGGGGCACAAGCUGGAGUACAACUACAACAGCCACAACGUCUAUAUCAU
GGCCGACAAGCAGAAGAACGGCAUCAAGGUGAACUUCAAGAUCCGCCACAACA
UCGAGGACGGCAGCGUGCAGCUCGCCGACCACUACCAGCAGAACACCCCCAUC
GGCGACGGCCCCGUGCUGCUGCCCGACAACCACUACCUGAGCACCCAGUCCGC
CCUGAGCAAAGACCCCAACGAGAAGCGCGAUCACAUGGUCCUGCUGGAGUUCG
UGACCGCCGCCGGGAUCACUCUCGGCAUGGACGAGCUGUACAAGUCUAGAUGA
UUGUGUAUGCGUUAAUAAAAAGAAGGAACUCGUA
T44-TOP-uAUG-TOM20-mCherry (Mitochondria targeting
mCherry mRNA)
(SEQ ID NO: 30)
GAUCCGCCAUCGUGGGUGAGUGUUAGCUCUGUGGCCGCGCUCUGGCUAGUGGC
GCUACGCGUCGCUCUCACGGGUGUCGUCGGAUCUAAUCCGUCUCUUUUCGAUA
GCAGGUGGAGCCGCCGCCACGAUGGUGGGACGGAACAGCGCCAUCGCUGCAGG
AGUGUGCGGUGCCCUCUUCAUAGGGUACUGCAUCUACUUUGACCGCAAAAGGA
GGAGUGACCCCAACCUCGAGGUGAGCAAGGGCGAGGAGGAUAACAUGGCCAUC
AUCAAGGAGUUCAUGCGCUUCAAGGUGCACAUGGAGGGCUCCGUGAACGGCCA
CGAGUUCGAGAUCGAGGGCGAGGGCGAGGGCCGCCCCUACGAGGGCACCCAGA
CCGCCAAGCUGAAGGUGACCAAGGGUGGCCCCCUGCCCUUCGCCUGGGACAUC
CUGUCCCCUCAGUUCAUGUACGGCUCCAAGGCCUACGUGAAGCACCCCGCCGA
CAUCCCCGACUACUUGAAGCUGUCCUUCCCCGAGGGCUUCAAGUGGGAGCGCG
UGAUGAACUUCGAGGACGGCGGCGUGGUGACCGUGACCCAGGACUCCUCCCUG
CAGGACGGCGAGUUCAUCUACAAGGUGAAGCUGCGCGGCACCAACUUCCCCUC
CGACGGCCCCGUAAUGCAGAAGAAGACCAUGGGCUGGGAGGCCUCCUCCGAGC
GGAUGUACCCCGAGGACGGCGCCCUGAAGGGCGAGAUCAAGCAGAGGCUGAAG
CUGAAGGACGGCGGCCACUACGACGCUGAGGUCAAGACCACCUACAAGGCCAA
GAAGCCCGUGCAGCUGCCCGGCGCCUACAACGUCAACAUCAAGUUGGACAUCA
CCUCCCACAACGAGGACUACACCAUCGUGGAACAGUACGAACGCGCCGAGGGC
CGCCACUCCACCGGCGGCAUGGACGAGCUGUACAAGUCUAGAUGAUUGUGUAU
GCGUUAAUAAAAAGAAGGAACUCGUA
T44-TOP-uAUG-CatB-EGFP (Lysosome targeting eGFP mRNA)
(SEQ ID NO: 31)
GAUCCGCCAUCGUGGGUGAGUGUUAGCUCUGUGGCCGCGCUCUGGCUAGUGGC
GCUACGCGUCGCUCUCACGGGUGUCGUCGGAUCUAAUCCGUCUCUUUUCGAUA
GCAGGUGGAGCCGCCGCCACGAUGUGGUGGUCCUUGAUCCUUCUUUCUUGCCU
GCUGGCACUGACCAGUGCCCAUGACAAGCCUUCCUUCCACCCGCUGUCGGAUG
ACCUGAUUAACUAUAUCAACAAACAGAAUACAACAUGGCAGGCUGGACGCAAC
UUCUACAAUGUUGACAUAAGCUAUCUGAAGAAGCUGUGUGGCACUGUCCUGG
GUGGACCCAAACUGCCAGGAAGGGUUGCGUUCGGUGAGGACAUAGAUCUACC
UGAAACCUUUGAUGCACGGGAACAAUGGUCCAACUGCCCGACCAUUGGACAGA
UUAGAGACCAGGGCUCCUGCGGCUCUUGUUGGGCAUUUGGGGCAGUGGAAGC
CAUUUCUGACCGAACCUGCAUUCACACCAAUGGCCGAGUCAACGUGGAGGUGU
CUGCUGAAGACCUGCUUACUUGCUGUGGUAUCCAGUGUGGGGACGGCUGUAA
UGGUGGCUAUCCCUCUGGAGCAUGGAGCUUCUGGACAAAAAAAGGCCUGGUU
UCAGGUGGAGUCUACAAUUCUCAUGUAGGCUGCUUACCAUACACCAUCCCUCC
CUGCGAGCACCAUGUCAAUGGCUCCCGUCCCCCAUGCACUGGAGAAGGAGAUA
CUCCCAGGUGCAACAAGAGCUGUGAAGCUGGCUACUCCCCAUCCUACAAAGAG
GAUAAGCACUUUGGGUACACUUCCUACAGCGUGUCUAACAGUGUGAAGGAGA
UCAUGGCAGAAAUCUACAAAAAUGGCCCAGUGGAGGGUGCCUUCACUGUGUU
UUCUGACUUCUUGACUUACAAAUCAGGAGUAUACAAGCAUGAAGCCGGUGAU
AUGAUGGGUGGCCACGCCAUCCGCAUCCUGGGCUGGGGAGUAGAGAAUGGAG
UUCCCUACUGGCUGGCAGCCAACUCUUGGAACCUUGACUGGGGUGAUAAUGGC
UUCUUUAAAAUCCUCAGAGGAGAAAACCACUGUGGCAUUGAAUCAGAAAUUG
UGGCUGGAAUCCCACGCACUGACCAGUACUGGGGAAGAUUCGUGAGCAAGGGC
GAGGAGCUGUUCACCGGGGUGGUGCCCAUCCUGGUCGAGCUGGACGGCGACGU
AAACGGCCACAAGUUCAGCGUGUCCGGCGAGGGCGAGGGCGAUGCCACCUACG
GCAAGCUGACCCUGAAGUUCAUCUGCACCACCGGCAAGCUGCCCGUGCCCUGG
CCCACCCUCGUGACCACCCUGACCUACGGCGUGCAGUGCUUCAGCCGCUACCCC
GACCACAUGAAGCAGCACGACUUCUUCAAGUCCGCCAUGCCCGAAGGCUACGU
CCAGGAGCGCACCAUCUUCUUCAAGGACGACGGCAACUACAAGACCCGCGCCG
AGGUGAAGUUCGAGGGCGACACCCUGGUGAACCGCAUCGAGCUGAAGGGCAUC
GACUUCAAGGAGGACGGCAACAUCCUGGGGCACAAGCUGGAGUACAACUACAA
CAGCCACAACGUCUAUAUCAUGGCCGACAAGCAGAAGAACGGCAUCAAGGUGA
ACUUCAAGAUCCGCCACAACAUCGAGGACGGCAGCGUGCAGCUCGCCGACCAC
UACCAGCAGAACACCCCCAUCGGCGACGGCCCCGUGCUGCUGCCCGACAACCA
CUACCUGAGCACCCAGUCCGCCCUGAGCAAAGACCCCAACGAGAAGCGCGAUC
ACAUGGUCCUGCUGGAGUUCGUGACCGCCGCCGGGAUCACUCUCGGCAUGGAC
GAGCUGUACAAGUGAUUGUGUAUGCGUUAAUAAAAAGAAGGAACUCGUA
T44-TOP-uAUG-CatB-mCherry (Lysosome targeting mCherry mRNA)
(SEQ ID NO: 32)
GAUCCGCCAUCGUGGGUGAGUGUUAGCUCUGUGGCCGCGCUCUGGCUAGUGGC
GCUACGCGUCGCUCUCACGGGUGUCGUCGGAUCUAAUCCGUCUCUUUUCGAUA
GCAGGUGGAGCCGCCGCCACGAUGUGGUGGUCCUUGAUCCUUCUUUCUUGCCU
GCUGGCACUGACCAGUGCCCAUGACAAGCCUUCCUUCCACCCGCUGUCGGAUG
ACCUGAUUAACUAUAUCAACAAACAGAAUACAACAUGGCAGGCUGGACGCAAC
UUCUACAAUGUUGACAUAAGCUAUCUGAAGAAGCUGUGUGGCACUGUCCUGG
GUGGACCCAAACUGCCAGGAAGGGUUGCGUUCGGUGAGGACAUAGAUCUACC
UGAAACCUUUGAUGCACGGGAACAAUGGUCCAACUGCCCGACCAUUGGACAGA
UUAGAGACCAGGGCUCCUGCGGCUCUUGUUGGGCAUUUGGGGCAGUGGAAGC
CAUUUCUGACCGAACCUGCAUUCACACCAAUGGCCGAGUCAACGUGGAGGUGU
CUGCUGAAGACCUGCUUACUUGCUGUGGUAUCCAGUGUGGGGACGGCUGUAA
UGGUGGCUAUCCCUCUGGAGCAUGGAGCUUCUGGACAAAAAAAGGCCUGGUU
UCAGGUGGAGUCUACAAUUCUCAUGUAGGCUGCUUACCAUACACCAUCCCUCC
CUGCGAGCACCAUGUCAAUGGCUCCCGUCCCCCAUGCACUGGAGAAGGAGAUA
CUCCCAGGUGCAACAAGAGCUGUGAAGCUGGCUACUCCCCAUCCUACAAAGAG
GAUAAGCACUUUGGGUACACUUCCUACAGCGUGUCUAACAGUGUGAAGGAGA
UCAUGGCAGAAAUCUACAAAAAUGGCCCAGUGGAGGGUGCCUUCACUGUGUU
UUCUGACUUCUUGACUUACAAAUCAGGAGUAUACAAGCAUGAAGCCGGUGAU
AUGAUGGGUGGCCACGCCAUCCGCAUCCUGGGCUGGGGAGUAGAGAAUGGAG
UUCCCUACUGGCUGGCAGCCAACUCUUGGAACCUUGACUGGGGUGAUAAUGGC
UUCUUUAAAAUCCUCAGAGGAGAAAACCACUGUGGCAUUGAAUCAGAAAUUG
UGGCUGGAAUCCCACGCACUGACCAGUACUGGGGAAGAUUCGUGAGCAAGGGC
GAGGAGGAUAACAUGGCCAUCAUCAAGGAGUUCAUGCGCUUCAAGGUGCACA
UGGAGGGCUCCGUGAACGGCCACGAGUUCGAGAUCGAGGGCGAGGGCGAGGG
CCGCCCCUACGAGGGCACCCAGACCGCCAAGCUGAAGGUGACCAAGGGUGGCC
CCCUGCCCUUCGCCUGGGACAUCCUGUCCCCUCAGUUCAUGUACGGCUCCAAG
GCCUACGUGAAGCACCCCGCCGACAUCCCCGACUACUUGAAGCUGUCCUUCCC
CGAGGGCUUCAAGUGGGAGCGCGUGAUGAACUUCGAGGACGGCGGCGUGGUG
ACCGUGACCCAGGACUCCUCCCUGCAGGACGGCGAGUUCAUCUACAAGGUGAA
GCUGCGCGGCACCAACUUCCCCUCCGACGGCCCCGUAAUGCAGAAGAAGACCA
UGGGCUGGGAGGCCUCCUCCGAGCGGAUGUACCCCGAGGACGGCGCCCUGAAG
GGCGAGAUCAAGCAGAGGCUGAAGCUGAAGGACGGCGGCCACUACGACGCUGA
GGUCAAGACCACCUACAAGGCCAAGAAGCCCGUGCAGCUGCCCGGCGCCUACA
ACGUCAACAUCAAGUUGGACAUCACCUCCCACAACGAGGACUACACCAUCGUG
GAACAGUACGAACGCGCCGAGGGCCGCCACUCCACCGGCGGCAUGGACGAGCU
GUACAAGUGAUUGUGUAUGCGUUAAUAAAAAGAAGGAACUCGUA
T44-top-uAUG-NLS-eGFP-NLS (Nucleus targeting eGFP mRNA)
(SEQ ID NO: 33)
GAUCCGCCAUCGUGGGUGAGUGUUAGCUCUGUGGCCGCGCUCUGGCUAGUGGC
GCUACGCGUCGCUCUCACGGGUGUCGUCGGAUCUAAUCCGUCUCUUUUCGAUA
GCAGGUGGAGCCGCCGCCACGAUGGCCCCAAAGAAGAAGCGGAAGGUCGGUAU
CCACGGAGUCCCAGCAGCCGUGAGCAAGGGCGAGGAGCUGUUCACCGGGGUGG
UGCCCAUCCUGGUCGAGCUGGACGGCGACGUAAACGGCCACAAGUUCAGCGUG
UCCGGCGAGGGCGAGGGCGAUGCCACCUACGGCAAGCUGACCCUGAAGUUCAU
CUGCACCACCGGCAAGCUGCCCGUGCCCUGGCCCACCCUCGUGACCACCCUGAC
CUACGGCGUGCAGUGCUUCAGCCGCUACCCCGACCACAUGAAGCAGCACGACU
UCUUCAAGUCCGCCAUGCCCGAAGGCUACGUCCAGGAGCGCACCAUCUUCUUC
AAGGACGACGGCAACUACAAGACCCGCGCCGAGGUGAAGUUCGAGGGCGACAC
CCUGGUGAACCGCAUCGAGCUGAAGGGCAUCGACUUCAAGGAGGACGGCAACA
UCCUGGGGCACAAGCUGGAGUACAACUACAACAGCCACAACGUCUAUAUCAUG
GCCGACAAGCAGAAGAACGGCAUCAAGGUGAACUUCAAGAUCCGCCACAACAU
CGAGGACGGCAGCGUGCAGCUCGCCGACCACUACCAGCAGAACACCCCCAUCG
GCGACGGCCCCGUGCUGCUGCCCGACAACCACUACCUGAGCACCCAGUCCGCCC
UGAGCAAAGACCCCAACGAGAAGCGCGAUCACAUGGUCCUGCUGGAGUUCGUG
ACCGCCGCCGGGAUCACUCUCGGCAUGGACGAGCUGUACAAGAAGCGUCCUGC
UGCUACUAAGAAAGCUGGUCAAGCUAAGAAAAAGAAAUAAGCGGCCGCUUGU
GUAUGCGUUAAUAAAAAGAAGGAACUCGUA
T44-top-uAUG-NLS-mCherry-NLS (Nucleus targeting mCherry mRNA)
(SEQ ID NO: 34)
GGGGAUCCGCCAUCGUGGGUGAGUGUUAGCUCUGUGGCCGCGCUCUGGCUAGU
GGCGCUACGCGUCGCUCUCACGGGUGUCGUCGGAUCUAAUCCGUCUCUUUUCG
AUAGCAGGUGGAGCCGCCGCCACGAUGGCCCCAAAGAAGAAGCGGAAGGUCGG
UAUCCACGGAGUCCCAGCAGCCGUGAGCAAGGGCGAGGAGGAUAACAUGGCCA
UCAUCAAGGAGUUCAUGCGCUUCAAGGUGCACAUGGAGGGCUCCGUGAACGGC
CACGAGUUCGAGAUCGAGGGCGAGGGCGAGGGCCGCCCCUACGAGGGCACCCA
GACCGCCAAGCUGAAGGUGACCAAGGGUGGCCCCCUGCCCUUCGCCUGGGACA
UCCUGUCCCCUCAGUUCAUGUACGGCUCCAAGGCCUACGUGAAGCACCCCGCC
GACAUCCCCGACUACUUGAAGCUGUCCUUCCCCGAGGGCUUCAAGUGGGAGCG
CGUGAUGAACUUCGAGGACGGCGGCGUGGUGACCGUGACCCAGGACUCCUCCC
UGCAGGACGGCGAGUUCAUCUACAAGGUGAAGCUGCGCGGCACCAACUUCCCC
UCCGACGGCCCCGUAAUGCAGAAGAAGACCAUGGGCUGGGAGGCCUCCUCCGA
GCGGAUGUACCCCGAGGACGGCGCCCUGAAGGGCGAGAUCAAGCAGAGGCUGA
AGCUGAAGGACGGCGGCCACUACGACGCUGAGGUCAAGACCACCUACAAGGCC
AAGAAGCCCGUGCAGCUGCCCGGCGCCUACAACGUCAACAUCAAGUUGGACAU
CACCUCCCACAACGAGGACUACACCAUCGUGGAACAGUACGAACGCGCCGAGG
GCCGCCACUCCACCGGCGGCAUGGACGAGCUGUACAAGAAGCGUCCUGCUGCU
ACUAAGAAAGCUGGUCAAGCUAAGAAAAAGAAAUAAGCGGCCGCUUGUGUAU
GCGUUAAUAAAAAGAAGGAACUCGUA
T44-TOP-uAUG-TOM20-mCherry-P2A-Calnexin-eGFP
(SEQ ID NO: 35)
GAUCCGCCAUCGUGGGUGAGUGUUAGCUCUGUGGCCGCGCUCUGGCUAGUGGC
GCUACGCGUCGCUCUCACGGGUGUCGUCGGAUCUAAUCCGUCUCUUUUCGAUA
GCAGGUGGAGCCGCCGCCACGAUGGUGGGACGGAACAGCGCCAUCGCUGCAGG
AGUGUGCGGUGCCCUCUUCAUAGGGUACUGCAUCUACUUUGACCGCAAAAGGA
GGAGUGACCCCAACCUCGAGGUGAGCAAGGGCGAGGAGGAUAACAUGGCCAUC
AUCAAGGAGUUCAUGCGCUUCAAGGUGCACAUGGAGGGCUCCGUGAACGGCCA
CGAGUUCGAGAUCGAGGGCGAGGGCGAGGGCCGCCCCUACGAGGGCACCCAGA
CCGCCAAGCUGAAGGUGACCAAGGGUGGCCCCCUGCCCUUCGCCUGGGACAUC
CUGUCCCCUCAGUUCAUGUACGGCUCCAAGGCCUACGUGAAGCACCCCGCCGA
CAUCCCCGACUACUUGAAGCUGUCCUUCCCCGAGGGCUUCAAGUGGGAGCGCG
UGAUGAACUUCGAGGACGGCGGCGUGGUGACCGUGACCCAGGACUCCUCCCUG
CAGGACGGCGAGUUCAUCUACAAGGUGAAGCUGCGCGGCACCAACUUCCCCUC
CGACGGCCCCGUAAUGCAGAAGAAGACCAUGGGCUGGGAGGCCUCCUCCGAGC
GGAUGUACCCCGAGGACGGCGCCCUGAAGGGCGAGAUCAAGCAGAGGCUGAAG
CUGAAGGACGGCGGCCACUACGACGCUGAGGUCAAGACCACCUACAAGGCCAA
GAAGCCCGUGCAGCUGCCCGGCGCCUACAACGUCAACAUCAAGUUGGACAUCA
CCUCCCACAACGAGGACUACACCAUCGUGGAACAGUACGAACGCGCCGAGGGC
CGCCACUCCACCGGCGGCAUGGACGAGCUGUACAAGGGAUCCGGCGCAACAAA
CUUCUCUCUGCUGAAACAAGCCGGAGAUGUCGAAGAGAAUCCUGGACCGAUGG
AAGGGAAGUGGUUGCUGUGUAUGUUACUGGUGCUUGGAACUGCUAUUGUUGA
GGCUCAUGAUGGACAUGAUGAUGAUGUGAUUGAUAUUGAGGAUGACCUUGAC
GAUGUCAUUGAAGAGGUAGAAGACUCAAAACCAGAUACCACUGCUCCUCCUUC
AUCUCCCAAGGUUACUUACAAAGCUCCAGUUCCAACAGGGGAAGUAUAUUUU
GCUGAUUCUUUUGACAGAGGAACUCUGUCAGGGUGGAUUUUAUCCAAAGCCA
AGAAAGACGAUACCGAUGAUGAAAUUGCCAAAUAUGAUGGAAAGUGGGAGGU
AGAGGAAAUGAAGGAGUCAAAGCUUCCAGGUGAUAAAGGACUUGUGUUGAUG
UCUCGGGCCAAGCAUCAUGCCAUCUCUGCUAAACUGAACAAGCCCUUCCUGUU
UGACACCAAGCCUCUCAUUGUUCAGUAUGAGGUUAAUUUCCAAAAUGGAAUA
GAAUGUGGUGGUGCCUAUGUGAAACUGCUUUCUAAAACACCAGAACUCAACC
UGGAUCAGUUCCAUGACAAGACCCCUUAUACGAUUAUGUUUGGUCCAGAUAA
AUGUGGAGAGGACUAUAAACUGCACUUCAUCUUCCGACACAAAAACCCCAAAA
CGGGUAUCUAUGAAGAAAAACAUGCUAAGAGGCCAGAUGCAGAUCUGAAGAC
CUAUUUUACUGAUAAGAAAACACAUCUUUACACACUAAUCUUGAAUCCAGAU
AAUAGUUUUGAAAUACUGGUUGACCAAUCUGUGGUGAAUAGUGGAAAUCUGC
UCAAUGACAUGACUCCUCCUGUAAAUCCUUCACGUGAAAUUGAGGACCCAGAA
GACCGGAAGCCCGAGGAUUGGGAUGAAAGACCAAAAAUCCCAGAUCCAGAAGC
UGUCAAGCCAGAUGACUGGGAUGAAGAUGCCCCUGCUAAGAUUCCAGAUGAA
GAGGCCACAAAACCCGAAGGCUGGUUAGAUGAUGAGCCUGAGUACGUACCUG
AUCCAGACGCAGAGAAACCUGAGGAUUGGGAUGAAGACAUGGAUGGAGAAUG
GGAGGCUCCUCAGAUUGCCAACCCUAGAUGUGAGUCAGCUCCUGGAUGUGGUG
UCUGGCAGCGACCUGUGAUUGACAACCCCAAUUAUAAAGGCAAAUGGAAGCCU
CCUAUGAUUGACAAUCCCAGUUACCAGGGAAUCUGGAAACCCAGGAAAAUACC
AAAUCCAGAUUUCUUUGAAGAUCUGGAACCUUUCAGAAUGACUCCUUUUAGU
GCUAUUGGUUUGGAGCUGUGGUCCAUGACCUCUGACAUUUUUUUUGACAACU
UUAUCAUUUGUGCUGAUCGAAGAAUAGUUGAUGAUUGGGCCAAUGAUGGAUG
GGGCCUGAAGAAAGCUGCUGAUGGGGCUGCUGAGCCAGGCGUUGUGGGGCAG
AUGAACGAGGCAGCUGAAGAGCGCCCGUGGCUGUGGGUAGUCUAUAUUCUAA
CUGUAGCCCUUCCUGUGUUCCUGGUUAUCCUCUUCUGCUGUUCUGGAAAGAAA
CAGACCAGUGGUAUGGAGUAUAAGAAAACUGAUGCACCUCAACCGGAUGUGA
AGGAAGAGGAAGAAGAGAAGGAAGAGGAAAAGGACAAGGGAGAUGAGGAGGA
GGAAGGAGAAGAGAAACUUGAAGAGAAACAGAAAAGUGAUGCUGAAGAAGAU
GGUGGCACUGUCAGUCAAGAGGAGGAAGACAGAAAACCUAAAGCAGAGGAGG
AUGAAAUUUUGAACAGAUCACCAAGAAACAGAAAGCCACGAAGAGAGGUGAG
CAAGGGCGAGGAGCUGUUCACCGGGGUGGUGCCCAUCCUGGUCGAGCUGGACG
GCGACGUAAACGGCCACAAGUUCAGCGUGUCCGGCGAGGGCGAGGGCGAUGCC
ACCUACGGCAAGCUGACCCUGAAGUUCAUCUGCACCACCGGCAAGCUGCCCGU
GCCCUGGCCCACCCUCGUGACCACCCUGACCUACGGCGUGCAGUGCUUCAGCC
GCUACCCCGACCACAUGAAGCAGCACGACUUCUUCAAGUCCGCCAUGCCCGAA
GGCUACGUCCAGGAGCGCACCAUCUUCUUCAAGGACGACGGCAACUACAAGAC
CCGCGCCGAGGUGAAGUUCGAGGGCGACACCCUGGUGAACCGCAUCGAGCUGA
AGGGCAUCGACUUCAAGGAGGACGGCAACAUCCUGGGGCACAAGCUGGAGUAC
AACUACAACAGCCACAACGUCUAUAUCAUGGCCGACAAGCAGAAGAACGGCAU
CAAGGUGAACUUCAAGAUCCGCCACAACAUCGAGGACGGCAGCGUGCAGCUCG
CCGACCACUACCAGCAGAACACCCCCAUCGGCGACGGCCCCGUGCUGCUGCCCG
ACAACCACUACCUGAGCACCCAGUCCGCCCUGAGCAAAGACCCCAACGAGAAG
CGCGAUCACAUGGUCCUGCUGGAGUUCGUGACCGCCGCCGGGAUCACUCUCGG
CAUGGACGAGCUGUACAAGUGAUUGUGUAUGCGUUAAUAAAAAGAAGGAACU
CGUA
T44-TOP-uAUG-TOM20-mCherry-P2A-CatB-eGFP
(SEQ ID NO: 36)
GAUCCGCCAUCGUGGGUGAGUGUUAGCUCUGUGGCCGCGCUCUGGCUAGUGGC
GCUACGCGUCGCUCUCACGGGUGUCGUCGGAUCUAAUCCGUCUCUUUUCGAUA
GCAGGUGGAGCCGCCGCCACGAUGGUGGGACGGAACAGCGCCAUCGCUGCAGG
AGUGUGCGGUGCCCUCUUCAUAGGGUACUGCAUCUACUUUGACCGCAAAAGGA
GGAGUGACCCCAACCUCGAGGUGAGCAAGGGCGAGGAGGAUAACAUGGCCAUC
AUCAAGGAGUUCAUGCGCUUCAAGGUGCACAUGGAGGGCUCCGUGAACGGCCA
CGAGUUCGAGAUCGAGGGCGAGGGCGAGGGCCGCCCCUACGAGGGCACCCAGA
CCGCCAAGCUGAAGGUGACCAAGGGUGGCCCCCUGCCCUUCGCCUGGGACAUC
CUGUCCCCUCAGUUCAUGUACGGCUCCAAGGCCUACGUGAAGCACCCCGCCGA
CAUCCCCGACUACUUGAAGCUGUCCUUCCCCGAGGGCUUCAAGUGGGAGCGCG
UGAUGAACUUCGAGGACGGCGGCGUGGUGACCGUGACCCAGGACUCCUCCCUG
CAGGACGGCGAGUUCAUCUACAAGGUGAAGCUGCGCGGCACCAACUUCCCCUC
CGACGGCCCCGUAAUGCAGAAGAAGACCAUGGGCUGGGAGGCCUCCUCCGAGC
GGAUGUACCCCGAGGACGGCGCCCUGAAGGGCGAGAUCAAGCAGAGGCUGAAG
CUGAAGGACGGCGGCCACUACGACGCUGAGGUCAAGACCACCUACAAGGCCAA
GAAGCCCGUGCAGCUGCCCGGCGCCUACAACGUCAACAUCAAGUUGGACAUCA
CCUCCCACAACGAGGACUACACCAUCGUGGAACAGUACGAACGCGCCGAGGGC
CGCCACUCCACCGGCGGCAUGGACGAGCUGUACAAGGGAUCCGGCGCAACAAA
CUUCUCUCUGCUGAAACAAGCCGGAGAUGUCGAAGAGAAUCCUGGACCGAUGU
GGUGGUCCUUGAUCCUUCUUUCUUGCCUGCUGGCACUGACCAGUGCCCAUGAC
AAGCCUUCCUUCCACCCGCUGUCGGAUGACCUGAUUAACUAUAUCAACAAACA
GAAUACAACAUGGCAGGCUGGACGCAACUUCUACAAUGUUGACAUAAGCUAU
CUGAAGAAGCUGUGUGGCACUGUCCUGGGUGGACCCAAACUGCCAGGAAGGG
UUGCGUUCGGUGAGGACAUAGAUCUACCUGAAACCUUUGAUGCACGGGAACA
AUGGUCCAACUGCCCGACCAUUGGACAGAUUAGAGACCAGGGCUCCUGCGGCU
CUUGUUGGGCAUUUGGGGCAGUGGAAGCCAUUUCUGACCGAACCUGCAUUCAC
ACCAAUGGCCGAGUCAACGUGGAGGUGUCUGCUGAAGACCUGCUUACUUGCUG
UGGUAUCCAGUGUGGGGACGGCUGUAAUGGUGGCUAUCCCUCUGGAGCAUGG
AGCUUCUGGACAAAAAAAGGCCUGGUUUCAGGUGGAGUCUACAAUUCUCAUG
UAGGCUGCUUACCAUACACCAUCCCUCCCUGCGAGCACCAUGUCAAUGGCUCC
CGUCCCCCAUGCACUGGAGAAGGAGAUACUCCCAGGUGCAACAAGAGCUGUGA
AGCUGGCUACUCCCCAUCCUACAAAGAGGAUAAGCACUUUGGGUACACUUCCU
ACAGCGUGUCUAACAGUGUGAAGGAGAUCAUGGCAGAAAUCUACAAAAAUGG
CCCAGUGGAGGGUGCCUUCACUGUGUUUUCUGACUUCUUGACUUACAAAUCAG
GAGUAUACAAGCAUGAAGCCGGUGAUAUGAUGGGUGGCCACGCCAUCCGCAUC
CUGGGCUGGGGAGUAGAGAAUGGAGUUCCCUACUGGCUGGCAGCCAACUCUU
GGAACCUUGACUGGGGUGAUAAUGGCUUCUUUAAAAUCCUCAGAGGAGAAAA
CCACUGUGGCAUUGAAUCAGAAAUUGUGGCUGGAAUCCCACGCACUGACCAGU
ACUGGGGAAGAUUCGUGAGCAAGGGCGAGGAGCUGUUCACCGGGGUGGUGCC
CAUCCUGGUCGAGCUGGACGGCGACGUAAACGGCCACAAGUUCAGCGUGUCCG
GCGAGGGCGAGGGCGAUGCCACCUACGGCAAGCUGACCCUGAAGUUCAUCUGC
ACCACCGGCAAGCUGCCCGUGCCCUGGCCCACCCUCGUGACCACCCUGACCUAC
GGCGUGCAGUGCUUCAGCCGCUACCCCGACCACAUGAAGCAGCACGACUUCUU
CAAGUCCGCCAUGCCCGAAGGCUACGUCCAGGAGCGCACCAUCUUCUUCAAGG
ACGACGGCAACUACAAGACCCGCGCCGAGGUGAAGUUCGAGGGCGACACCCUG
GUGAACCGCAUCGAGCUGAAGGGCAUCGACUUCAAGGAGGACGGCAACAUCCU
GGGGCACAAGCUGGAGUACAACUACAACAGCCACAACGUCUAUAUCAUGGCCG
ACAAGCAGAAGAACGGCAUCAAGGUGAACUUCAAGAUCCGCCACAACAUCGAG
GACGGCAGCGUGCAGCUCGCCGACCACUACCAGCAGAACACCCCCAUCGGCGA
CGGCCCCGUGCUGCUGCCCGACAACCACUACCUGAGCACCCAGUCCGCCCUGA
GCAAAGACCCCAACGAGAAGCGCGAUCACAUGGUCCUGCUGGAGUUCGUGACC
GCCGCCGGGAUCACUCUCGGCAUGGACGAGCUGUACAAGUGAUUGUGUAUGCG
UUAAUAAAAAGAAGGAACUCGUA
T44-TOP-uAUG-TOM20-mCherry-P2A-NLS-eGFP-NLS
(SEQ ID NO: 37)
GAUCCGCCAUCGUGGGUGAGUGUUAGCUCUGUGGCCGCGCUCUGGCUAGUGGC
GCUACGCGUCGCUCUCACGGGUGUCGUCGGAUCUAAUCCGUCUCUUUUCGAUA
GCAGGUGGAGCCGCCGCCACGAUGGUGGGACGGAACAGCGCCAUCGCUGCAGG
AGUGUGCGGUGCCCUCUUCAUAGGGUACUGCAUCUACUUUGACCGCAAAAGGA
GGAGUGACCCCAACCUCGAGGUGAGCAAGGGCGAGGAGGAUAACAUGGCCAUC
AUCAAGGAGUUCAUGCGCUUCAAGGUGCACAUGGAGGGCUCCGUGAACGGCCA
CGAGUUCGAGAUCGAGGGCGAGGGCGAGGGCCGCCCCUACGAGGGCACCCAGA
CCGCCAAGCUGAAGGUGACCAAGGGUGGCCCCCUGCCCUUCGCCUGGGACAUC
CUGUCCCCUCAGUUCAUGUACGGCUCCAAGGCCUACGUGAAGCACCCCGCCGA
CAUCCCCGACUACUUGAAGCUGUCCUUCCCCGAGGGCUUCAAGUGGGAGCGCG
UGAUGAACUUCGAGGACGGCGGCGUGGUGACCGUGACCCAGGACUCCUCCCUG
CAGGACGGCGAGUUCAUCUACAAGGUGAAGCUGCGCGGCACCAACUUCCCCUC
CGACGGCCCCGUAAUGCAGAAGAAGACCAUGGGCUGGGAGGCCUCCUCCGAGC
GGAUGUACCCCGAGGACGGCGCCCUGAAGGGCGAGAUCAAGCAGAGGCUGAAG
CUGAAGGACGGCGGCCACUACGACGCUGAGGUCAAGACCACCUACAAGGCCAA
GAAGCCCGUGCAGCUGCCCGGCGCCUACAACGUCAACAUCAAGUUGGACAUCA
CCUCCCACAACGAGGACUACACCAUCGUGGAACAGUACGAACGCGCCGAGGGC
CGCCACUCCACCGGCGGCAUGGACGAGCUGUACAAGGGAUCCGGCGCAACAAA
CUUCUCUCUGCUGAAACAAGCCGGAGAUGUCGAAGAGAAUCCUGGACCGAUGG
CCCCAAAGAAGAAGCGGAAGGUCGGUAUCCACGGAGUCCCAGCAGCCGUGAGC
AAGGGCGAGGAGCUGUUCACCGGGGUGGUGCCCAUCCUGGUCGAGCUGGACGG
CGACGUAAACGGCCACAAGUUCAGCGUGUCCGGCGAGGGCGAGGGCGAUGCCA
CCUACGGCAAGCUGACCCUGAAGUUCAUCUGCACCACCGGCAAGCUGCCCGUG
CCCUGGCCCACCCUCGUGACCACCCUGACCUACGGCGUGCAGUGCUUCAGCCG
CUACCCCGACCACAUGAAGCAGCACGACUUCUUCAAGUCCGCCAUGCCCGAAG
GCUACGUCCAGGAGCGCACCAUCUUCUUCAAGGACGACGGCAACUACAAGACC
CGCGCCGAGGUGAAGUUCGAGGGCGACACCCUGGUGAACCGCAUCGAGCUGAA
GGGCAUCGACUUCAAGGAGGACGGCAACAUCCUGGGGCACAAGCUGGAGUACA
ACUACAACAGCCACAACGUCUAUAUCAUGGCCGACAAGCAGAAGAACGGCAUC
AAGGUGAACUUCAAGAUCCGCCACAACAUCGAGGACGGCAGCGUGCAGCUCGC
CGACCACUACCAGCAGAACACCCCCAUCGGCGACGGCCCCGUGCUGCUGCCCG
ACAACCACUACCUGAGCACCCAGUCCGCCCUGAGCAAAGACCCCAACGAGAAG
CGCGAUCACAUGGUCCUGCUGGAGUUCGUGACCGCCGCCGGGAUCACUCUCGG
CAUGGACGAGCUGUACAAGAAGCGUCCUGCUGCUACUAAGAAAGCUGGUCAA
GCUAAGAAAAAGAAAUAAGCGGCCGCUUGUGUAUGCGUUAAUAAAAAGAAGG
AACUCGUA
T44-TOP-uAUG-TOM20-mCherry-GGGGS4-Calexin-eGFP
(SEQ ID NO: 38)
GAUCCGCCAUCGUGGGUGAGUGUUAGCUCUGUGGCCGCGCUCUGGCUAGUGGC
GCUACGCGUCGCUCUCACGGGUGUCGUCGGAUCUAAUCCGUCUCUUUUCGAUA
GCAGGUGGAGCCGCCGCCACGAUGGUGGGACGGAACAGCGCCAUCGCUGCAGG
AGUGUGCGGUGCCCUCUUCAUAGGGUACUGCAUCUACUUUGACCGCAAAAGGA
GGAGUGACCCCAACCUCGAGGUGAGCAAGGGCGAGGAGGAUAACAUGGCCAUC
AUCAAGGAGUUCAUGCGCUUCAAGGUGCACAUGGAGGGCUCCGUGAACGGCCA
CGAGUUCGAGAUCGAGGGCGAGGGCGAGGGCCGCCCCUACGAGGGCACCCAGA
CCGCCAAGCUGAAGGUGACCAAGGGUGGCCCCCUGCCCUUCGCCUGGGACAUC
CUGUCCCCUCAGUUCAUGUACGGCUCCAAGGCCUACGUGAAGCACCCCGCCGA
CAUCCCCGACUACUUGAAGCUGUCCUUCCCCGAGGGCUUCAAGUGGGAGCGCG
UGAUGAACUUCGAGGACGGCGGCGUGGUGACCGUGACCCAGGACUCCUCCCUG
CAGGACGGCGAGUUCAUCUACAAGGUGAAGCUGCGCGGCACCAACUUCCCCUC
CGACGGCCCCGUAAUGCAGAAGAAGACCAUGGGCUGGGAGGCCUCCUCCGAGC
GGAUGUACCCCGAGGACGGCGCCCUGAAGGGCGAGAUCAAGCAGAGGCUGAAG
CUGAAGGACGGCGGCCACUACGACGCUGAGGUCAAGACCACCUACAAGGCCAA
GAAGCCCGUGCAGCUGCCCGGCGCCUACAACGUCAACAUCAAGUUGGACAUCA
CCUCCCACAACGAGGACUACACCAUCGUGGAACAGUACGAACGCGCCGAGGGC
CGCCACUCCACCGGCGGCAUGGACGAGCUGUACAAGGGAGGUGGAGGCAGCGG
AGGCGGGGGCAGUGGAGGAGGGGGUUCCGGUGGUGGUGGUAGUAUGGAAGGG
AAGUGGUUGCUGUGUAUGUUACUGGUGCUUGGAACUGCUAUUGUUGAGGCUC
AUGAUGGACAUGAUGAUGAUGUGAUUGAUAUUGAGGAUGACCUUGACGAUGU
CAUUGAAGAGGUAGAAGACUCAAAACCAGAUACCACUGCUCCUCCUUCAUCUC
CCAAGGUUACUUACAAAGCUCCAGUUCCAACAGGGGAAGUAUAUUUUGCUGA
UUCUUUUGACAGAGGAACUCUGUCAGGGUGGAUUUUAUCCAAAGCCAAGAAA
GACGAUACCGAUGAUGAAAUUGCCAAAUAUGAUGGAAAGUGGGAGGUAGAGG
AAAUGAAGGAGUCAAAGCUUCCAGGUGAUAAAGGACUUGUGUUGAUGUCUCG
GGCCAAGCAUCAUGCCAUCUCUGCUAAACUGAACAAGCCCUUCCUGUUUGACA
CCAAGCCUCUCAUUGUUCAGUAUGAGGUUAAUUUCCAAAAUGGAAUAGAAUG
UGGUGGUGCCUAUGUGAAACUGCUUUCUAAAACACCAGAACUCAACCUGGAUC
AGUUCCAUGACAAGACCCCUUAUACGAUUAUGUUUGGUCCAGAUAAAUGUGG
AGAGGACUAUAAACUGCACUUCAUCUUCCGACACAAAAACCCCAAAACGGGUA
UCUAUGAAGAAAAACAUGCUAAGAGGCCAGAUGCAGAUCUGAAGACCUAUUU
UACUGAUAAGAAAACACAUCUUUACACACUAAUCUUGAAUCCAGAUAAUAGU
UUUGAAAUACUGGUUGACCAAUCUGUGGUGAAUAGUGGAAAUCUGCUCAAUG
ACAUGACUCCUCCUGUAAAUCCUUCACGUGAAAUUGAGGACCCAGAAGACCGG
AAGCCCGAGGAUUGGGAUGAAAGACCAAAAAUCCCAGAUCCAGAAGCUGUCA
AGCCAGAUGACUGGGAUGAAGAUGCCCCUGCUAAGAUUCCAGAUGAAGAGGC
CACAAAACCCGAAGGCUGGUUAGAUGAUGAGCCUGAGUACGUACCUGAUCCAG
ACGCAGAGAAACCUGAGGAUUGGGAUGAAGACAUGGAUGGAGAAUGGGAGGC
UCCUCAGAUUGCCAACCCUAGAUGUGAGUCAGCUCCUGGAUGUGGUGUCUGGC
AGCGACCUGUGAUUGACAACCCCAAUUAUAAAGGCAAAUGGAAGCCUCCUAUG
AUUGACAAUCCCAGUUACCAGGGAAUCUGGAAACCCAGGAAAAUACCAAAUCC
AGAUUUCUUUGAAGAUCUGGAACCUUUCAGAAUGACUCCUUUUAGUGCUAUU
GGUUUGGAGCUGUGGUCCAUGACCUCUGACAUUUUUUUUGACAACUUUAUCA
UUUGUGCUGAUCGAAGAAUAGUUGAUGAUUGGGCCAAUGAUGGAUGGGGCCU
GAAGAAAGCUGCUGAUGGGGCUGCUGAGCCAGGCGUUGUGGGGCAGAUGAAC
GAGGCAGCUGAAGAGCGCCCGUGGCUGUGGGUAGUCUAUAUUCUAACUGUAG
CCCUUCCUGUGUUCCUGGUUAUCCUCUUCUGCUGUUCUGGAAAGAAACAGACC
AGUGGUAUGGAGUAUAAGAAAACUGAUGCACCUCAACCGGAUGUGAAGGAAG
AGGAAGAAGAGAAGGAAGAGGAAAAGGACAAGGGAGAUGAGGAGGAGGAAGG
AGAAGAGAAACUUGAAGAGAAACAGAAAAGUGAUGCUGAAGAAGAUGGUGGC
ACUGUCAGUCAAGAGGAGGAAGACAGAAAACCUAAAGCAGAGGAGGAUGAAA
UUUUGAACAGAUCACCAAGAAACAGAAAGCCACGAAGAGAGGUGAGCAAGGG
CGAGGAGCUGUUCACCGGGGUGGUGCCCAUCCUGGUCGAGCUGGACGGCGACG
UAAACGGCCACAAGUUCAGCGUGUCCGGCGAGGGCGAGGGCGAUGCCACCUAC
GGCAAGCUGACCCUGAAGUUCAUCUGCACCACCGGCAAGCUGCCCGUGCCCUG
GCCCACCCUCGUGACCACCCUGACCUACGGCGUGCAGUGCUUCAGCCGCUACC
CCGACCACAUGAAGCAGCACGACUUCUUCAAGUCCGCCAUGCCCGAAGGCUAC
GUCCAGGAGCGCACCAUCUUCUUCAAGGACGACGGCAACUACAAGACCCGCGC
CGAGGUGAAGUUCGAGGGCGACACCCUGGUGAACCGCAUCGAGCUGAAGGGCA
UCGACUUCAAGGAGGACGGCAACAUCCUGGGGCACAAGCUGGAGUACAACUAC
AACAGCCACAACGUCUAUAUCAUGGCCGACAAGCAGAAGAACGGCAUCAAGGU
GAACUUCAAGAUCCGCCACAACAUCGAGGACGGCAGCGUGCAGCUCGCCGACC
ACUACCAGCAGAACACCCCCAUCGGCGACGGCCCCGUGCUGCUGCCCGACAAC
CACUACCUGAGCACCCAGUCCGCCCUGAGCAAAGACCCCAACGAGAAGCGCGA
UCACAUGGUCCUGCUGGAGUUCGUGACCGCCGCCGGGAUCACUCUCGGCAUGG
ACGAGCUGUACAAGUGAUUGUGUAUGCGUUAAUAAAAAGAAGGAACUCGUA
T44-TOP-uAUG-TOM20-mCherry-GGGGS4-CatB-eGFP
(SEQ ID NO: 39)
GAUCCGCCAUCGUGGGUGAGUGUUAGCUCUGUGGCCGCGCUCUGGCUAGUGGC
GCUACGCGUCGCUCUCACGGGUGUCGUCGGAUCUAAUCCGUCUCUUUUCGAUA
GCAGGUGGAGCCGCCGCCACGAUGGUGGGACGGAACAGCGCCAUCGCUGCAGG
AGUGUGCGGUGCCCUCUUCAUAGGGUACUGCAUCUACUUUGACCGCAAAAGGA
GGAGUGACCCCAACCUCGAGGUGAGCAAGGGCGAGGAGGAUAACAUGGCCAUC
AUCAAGGAGUUCAUGCGCUUCAAGGUGCACAUGGAGGGCUCCGUGAACGGCCA
CGAGUUCGAGAUCGAGGGCGAGGGCGAGGGCCGCCCCUACGAGGGCACCCAGA
CCGCCAAGCUGAAGGUGACCAAGGGUGGCCCCCUGCCCUUCGCCUGGGACAUC
CUGUCCCCUCAGUUCAUGUACGGCUCCAAGGCCUACGUGAAGCACCCCGCCGA
CAUCCCCGACUACUUGAAGCUGUCCUUCCCCGAGGGCUUCAAGUGGGAGCGCG
UGAUGAACUUCGAGGACGGCGGCGUGGUGACCGUGACCCAGGACUCCUCCCUG
CAGGACGGCGAGUUCAUCUACAAGGUGAAGCUGCGCGGCACCAACUUCCCCUC
CGACGGCCCCGUAAUGCAGAAGAAGACCAUGGGCUGGGAGGCCUCCUCCGAGC
GGAUGUACCCCGAGGACGGCGCCCUGAAGGGCGAGAUCAAGCAGAGGCUGAAG
CUGAAGGACGGCGGCCACUACGACGCUGAGGUCAAGACCACCUACAAGGCCAA
GAAGCCCGUGCAGCUGCCCGGCGCCUACAACGUCAACAUCAAGUUGGACAUCA
CCUCCCACAACGAGGACUACACCAUCGUGGAACAGUACGAACGCGCCGAGGGC
CGCCACUCCACCGGCGGCAUGGACGAGCUGUACAAGGGAGGUGGAGGCAGCGG
AGGCGGGGGCAGUGGAGGAGGGGGUUCCGGUGGUGGUGGUAGUAUGUGGUGG
UCCUUGAUCCUUCUUUCUUGCCUGCUGGCACUGACCAGUGCCCAUGACAAGCC
UUCCUUCCACCCGCUGUCGGAUGACCUGAUUAACUAUAUCAACAAACAGAAUA
CAACAUGGCAGGCUGGACGCAACUUCUACAAUGUUGACAUAAGCUAUCUGAA
GAAGCUGUGUGGCACUGUCCUGGGUGGACCCAAACUGCCAGGAAGGGUUGCG
UUCGGUGAGGACAUAGAUCUACCUGAAACCUUUGAUGCACGGGAACAAUGGU
CCAACUGCCCGACCAUUGGACAGAUUAGAGACCAGGGCUCCUGCGGCUCUUGU
UGGGCAUUUGGGGCAGUGGAAGCCAUUUCUGACCGAACCUGCAUUCACACCAA
UGGCCGAGUCAACGUGGAGGUGUCUGCUGAAGACCUGCUUACUUGCUGUGGU
AUCCAGUGUGGGGACGGCUGUAAUGGUGGCUAUCCCUCUGGAGCAUGGAGCU
UCUGGACAAAAAAAGGCCUGGUUUCAGGUGGAGUCUACAAUUCUCAUGUAGG
CUGCUUACCAUACACCAUCCCUCCCUGCGAGCACCAUGUCAAUGGCUCCCGUC
CCCCAUGCACUGGAGAAGGAGAUACUCCCAGGUGCAACAAGAGCUGUGAAGCU
GGCUACUCCCCAUCCUACAAAGAGGAUAAGCACUUUGGGUACACUUCCUACAG
CGUGUCUAACAGUGUGAAGGAGAUCAUGGCAGAAAUCUACAAAAAUGGCCCA
GUGGAGGGUGCCUUCACUGUGUUUUCUGACUUCUUGACUUACAAAUCAGGAG
UAUACAAGCAUGAAGCCGGUGAUAUGAUGGGUGGCCACGCCAUCCGCAUCCUG
GGCUGGGGAGUAGAGAAUGGAGUUCCCUACUGGCUGGCAGCCAACUCUUGGA
ACCUUGACUGGGGUGAUAAUGGCUUCUUUAAAAUCCUCAGAGGAGAAAACCA
CUGUGGCAUUGAAUCAGAAAUUGUGGCUGGAAUCCCACGCACUGACCAGUACU
GGGGAAGAUUCGUGAGCAAGGGCGAGGAGCUGUUCACCGGGGUGGUGCCCAU
CCUGGUCGAGCUGGACGGCGACGUAAACGGCCACAAGUUCAGCGUGUCCGGCG
AGGGCGAGGGCGAUGCCACCUACGGCAAGCUGACCCUGAAGUUCAUCUGCACC
ACCGGCAAGCUGCCCGUGCCCUGGCCCACCCUCGUGACCACCCUGACCUACGGC
GUGCAGUGCUUCAGCCGCUACCCCGACCACAUGAAGCAGCACGACUUCUUCAA
GUCCGCCAUGCCCGAAGGCUACGUCCAGGAGCGCACCAUCUUCUUCAAGGACG
ACGGCAACUACAAGACCCGCGCCGAGGUGAAGUUCGAGGGCGACACCCUGGUG
AACCGCAUCGAGCUGAAGGGCAUCGACUUCAAGGAGGACGGCAACAUCCUGGG
GCACAAGCUGGAGUACAACUACAACAGCCACAACGUCUAUAUCAUGGCCGACA
AGCAGAAGAACGGCAUCAAGGUGAACUUCAAGAUCCGCCACAACAUCGAGGAC
GGCAGCGUGCAGCUCGCCGACCACUACCAGCAGAACACCCCCAUCGGCGACGG
CCCCGUGCUGCUGCCCGACAACCACUACCUGAGCACCCAGUCCGCCCUGAGCA
AAGACCCCAACGAGAAGCGCGAUCACAUGGUCCUGCUGGAGUUCGUGACCGCC
GCCGGGAUCACUCUCGGCAUGGACGAGCUGUACAAGUGAUUGUGUAUGCGUU
AAUAAAAAGAAGGAACUCGUA
T44-TOP-uAUG-TOM20-mCherry-GGGGS4-NLS-eGFP-NLS
(SEQ ID NO: 40)
GAUCCGCCAUCGUGGGUGAGUGUUAGCUCUGUGGCCGCGCUCUGGCUAGUGGC
GCUACGCGUCGCUCUCACGGGUGUCGUCGGAUCUAAUCCGUCUCUUUUCGAUA
GCAGGUGGAGCCGCCGCCACGAUGGUGGGACGGAACAGCGCCAUCGCUGCAGG
AGUGUGCGGUGCCCUCUUCAUAGGGUACUGCAUCUACUUUGACCGCAAAAGGA
GGAGUGACCCCAACCUCGAGGUGAGCAAGGGCGAGGAGGAUAACAUGGCCAUC
AUCAAGGAGUUCAUGCGCUUCAAGGUGCACAUGGAGGGCUCCGUGAACGGCCA
CGAGUUCGAGAUCGAGGGCGAGGGCGAGGGCCGCCCCUACGAGGGCACCCAGA
CCGCCAAGCUGAAGGUGACCAAGGGUGGCCCCCUGCCCUUCGCCUGGGACAUC
CUGUCCCCUCAGUUCAUGUACGGCUCCAAGGCCUACGUGAAGCACCCCGCCGA
CAUCCCCGACUACUUGAAGCUGUCCUUCCCCGAGGGCUUCAAGUGGGAGCGCG
UGAUGAACUUCGAGGACGGCGGCGUGGUGACCGUGACCCAGGACUCCUCCCUG
CAGGACGGCGAGUUCAUCUACAAGGUGAAGCUGCGCGGCACCAACUUCCCCUC
CGACGGCCCCGUAAUGCAGAAGAAGACCAUGGGCUGGGAGGCCUCCUCCGAGC
GGAUGUACCCCGAGGACGGCGCCCUGAAGGGCGAGAUCAAGCAGAGGCUGAAG
CUGAAGGACGGCGGCCACUACGACGCUGAGGUCAAGACCACCUACAAGGCCAA
GAAGCCCGUGCAGCUGCCCGGCGCCUACAACGUCAACAUCAAGUUGGACAUCA
CCUCCCACAACGAGGACUACACCAUCGUGGAACAGUACGAACGCGCCGAGGGC
CGCCACUCCACCGGCGGCAUGGACGAGCUGUACAAGGGAGGUGGAGGCAGCGG
AGGCGGGGGCAGUGGAGGAGGGGGUUCCGGUGGUGGUGGUAGUAUGGCCCCA
AAGAAGAAGCGGAAGGUCGGUAUCCACGGAGUCCCAGCAGCCGUGAGCAAGG
GCGAGGAGCUGUUCACCGGGGUGGUGCCCAUCCUGGUCGAGCUGGACGGCGAC
GUAAACGGCCACAAGUUCAGCGUGUCCGGCGAGGGCGAGGGCGAUGCCACCUA
CGGCAAGCUGACCCUGAAGUUCAUCUGCACCACCGGCAAGCUGCCCGUGCCCU
GGCCCACCCUCGUGACCACCCUGACCUACGGCGUGCAGUGCUUCAGCCGCUAC
CCCGACCACAUGAAGCAGCACGACUUCUUCAAGUCCGCCAUGCCCGAAGGCUA
CGUCCAGGAGCGCACCAUCUUCUUCAAGGACGACGGCAACUACAAGACCCGCG
CCGAGGUGAAGUUCGAGGGCGACACCCUGGUGAACCGCAUCGAGCUGAAGGGC
AUCGACUUCAAGGAGGACGGCAACAUCCUGGGGCACAAGCUGGAGUACAACUA
CAACAGCCACAACGUCUAUAUCAUGGCCGACAAGCAGAAGAACGGCAUCAAGG
UGAACUUCAAGAUCCGCCACAACAUCGAGGACGGCAGCGUGCAGCUCGCCGAC
CACUACCAGCAGAACACCCCCAUCGGCGACGGCCCCGUGCUGCUGCCCGACAA
CCACUACCUGAGCACCCAGUCCGCCCUGAGCAAAGACCCCAACGAGAAGCGCG
AUCACAUGGUCCUGCUGGAGUUCGUGACCGCCGCCGGGAUCACUCUCGGCAUG
GACGAGCUGUACAAGAAGCGUCCUGCUGCUACUAAGAAAGCUGGUCAAGCUA
AGAAAAAGAAAUAAGCGGCCGCUUGUGUAUGCGUUAAUAAAAAGAAGGAACU
CGUA

### The DNA Sequences for the Above RNA Sequences are Also Disclosed Herein:

5UTR-1 (T44)
5′ UTR from transcript ENSMUST00000102844 of mouse ribosomal protein S27a gene
(Gene symbol: RPS27A)
(SEQ ID NO: 41)
GGGTTTCCGATCCGCCATCGTGGGTGAGTGTATGCTCTGTGGCCGCGCTCTGGCTAGTGGCGCTACGCGTCGCTCTCACGGGTG
TCGTCGGATCTAATCCGTCTCTTTTCGAATGCAGGTGGAGCCGCCGCCACG
5UTR-2 (T44-top)
Modification of 5UTR-1 with 5′ terminal oligopyrimidine tract (5′ TOP) removed
(SEQ ID NO: 42)
GGGGATCCGCCATCGTGGGTGAGTGTATGCTCTGTGGCCGCGCTCTGGCTAGTGGCGCTACGCGTCGCTCTCACGGGTGTCGTC
GGATCTAATCCGTCTCTTTTCGAATGCAGGTGGAGCCGCCGCCACG
5UTR-3 (T44-top-uATG)
Modification of 5UTR-2: two upstream translation start codons ATG modified to TAG
(SEQ ID NO: 43)
GGGGATCCGCCATCGTGGGTGAGTGTtagCTCTGTGGCCGCGCTCTGGCTAGTGGCGCTACGCGTCGCTCTCACGGGTGTCGT
CGGATCTAATCCGTCTCTTTTCGAtagCAGGTGGAGCCGCCGCCACG
5UTR-4 (Truncated-T44-top-uATG)
Modification of 5UTR-3 with the first 83 nucleotides after GGG truncated
(SEQ ID NO: 44)
GGGATCTAATCCGTCTCTTTTCGATAGCAGGTGGAGCCGCCGCCACG
5UTR-5 (Truncated-T44-top-uATG-2ATG)
Modification of 5UTR-4 with one additional ATG added before the ATG in coding
region, resulting two tandem ATG translation start codons
(SEQ ID NO: 45)
GGGATCTAATCCGTCTCTTTTCGATAGCAGGTGGAGCCGCCGCCACGATG
5UTR-6 (T45)
5′UTR from transcript ENSMUST00000102845 of mouse ribosomal protein 527a gene
(Gene symbol: RPS27A)
(SEQ ID NO: 46)
GGGAGGAAAGCCTCTCTTAATCGCATCGGCTGTATAAGAAAGCCTTTTGAGGCATTTTTTTTAGTTGAGCACATCATTTCGAGG
CCATTCTGAGGTAAACCGAGAAAAGAGCGTAAAGAAACCGAGCGAACGAGCAAATCTGGCACTGCGTTAGACAGCCGCGATTCC
GCTGCAGCGCGCAGGCACGTGTGTGGCCGCCTAAGGGGCGGGTCCTTCGGCCAGGAGACCCCGTCGGCCACGCTCGGATCTTCC
TTTCCGATCCGCCATCGTGGGTGGAGCCGCCGCCACG
5UTR-7 (T45-top)
Modification of 5UTR-6 with 5′ terminal oligopyrimidine tract (5′ TOP) removed
(SEQ ID NO: 47)
GGGAGGAAAGAATCGCATCGGCTGTATAAGAAAGCCTTTTGAGGCATTTTTTTTAGTTGAGCACATCATTTCGAGGCCATTCTG
AGGTAAACCGAGAAAAGAGCGTAAAGAAACCGAGCGAACGAGCAAATCTGGCACTGCGTTAGACAGCCGCGATTCCGCTGCAGC
GCGCAGGCACGTGTGTGGCCGCCTAAGGGGCGGGTCCTTCGGCCAGGAGACCCCGTCGGCCACGCTCGGATCTTCCTTTCCGAT
CCGCCATCGTGGGTGGAGCCGCCGCCACG
5UTR-8 (T17)
5′UTR from transcript EN5T00000272317 of human ribosomal protein 527a gene
(Gene symbol: RPS27A)
(SEQ ID NO: 48)
GGGCCCCTCGACCTCCTTTTAAAAATTCTCTTAGCCACGTTGATTGTACGGGAAAAGCCTTTTTAAAACATCTTTTACGTTGCT
TAAACCTACAGTTTCGAAAGCATTCCGAAGGCTAAAGTGAGAAATAAGCCCAGGCTAGGGAGAGGAGAAACGAAGTTCACGTCC
TAGTCTGGCACCGGGTTGGATTGTCGCTGGGACGGCAGTCAGGCATTTGGTGTGGTCGCCTAAGGGGTGGGTCCTTCGGCGGGA
GCTCCGGGAAACCCCGTGGGCCTGCGCGGCGTTCTTCCTTTTCGATCCGCCATCTGCGGTGGAGCCGCCACCAAA
5UTR-9 (T17-TOP)
Modification of 5UTR-8 with 5′ terminal oligopyrimidine tract (5′ TOP) removed
(SEQ ID NO: 49)
GGGAGCCACGTTGATTGTACGGGAAAAGCCTTTTTAAAACATCTTTTACGTTGCTTAAACCTACAGTTTCGAAAGCATTCCGAA
GGCTAAAGTGAGAAATAAGCCCAGGCTAGGGAGAGGAGAAACGAAGTTCACGTCCTAGTCTGGCACCGGGTTGGATTGTCGCTG
GGACGGCAGTCAGGCATTTGGTGTGGTCGCCTAAGGGGTGGGTCCTTCGGCGGGAGCTCCGGGAAACCCCGTGGGCCTGCGCGG
CGTTCTTCCTTTTCGATCCGCCATCTGCGGTGGAGCCGCCACCAAA
5UTR-10 (T35)
5′UTR from transcript EN5T00000404735 of human ribosomal protein 527a gene
(Gene symbol: RPS27A)
(SEQ ID NO: 50)
GGGCGTTCTTCCTTTTCGATCCGCCATCTGCGGTGGGTGTCTGCACTTCGGCTGCTCTCGGGTTAGCACCCTATGGTGCCTTCT
CTTGTGATCCCTGACCTAACCTGTCTCTTCCTTTTCCTCAACCTCAGGTGGAGCCGCCACCAAA
5UTR-11 (T35-TOP)
Modification of 5UTR-10 with 5′ terminal oligopyrimidine tract (5′ TOP) removed
(SEQ ID NO: 51)
GGGCGCGATCCGCCATCTGCGGTGGGTGTCTGCACTTCGGCTGCTCTCGGGTTAGCACCCTATGGTGCCTTCTCTTGTGATCCC
TGACCTAACCTGTCTCTTCCTTTTCCTCAACCTCAGGTGGAGCCGCCACCAAA
5UTR-12 (10nt)
10nt unnatural 5′ UTR with GGG, kozak sequence (GCCACC) and minimal secondary
structure
(SEQ ID NO: 52)
GGGAGCCACC
5UTR-13 (20nt)
20nt unnatural 5′ UTR with GGG, kozak sequence (GCCACC) and minimal secondary
structure
(SEQ ID NO: 53)
GGGGACAGAAAACAGCCACC
5UTR-14 (30nt)
30nt unnatural 5′ UTR with GGG, kozak sequence (GCCACC) and minimal secondary
structure
(SEQ ID NO: 54)
GGGAAAGAAACAGGACAGAAAACAGCCACC
5UTR-15 (40nt)
40nt unnatural 5′ UTR with GGG, kozak sequence (GCCACC) and minimal secondary
structure
(SEQ ID NO: 55)
GGGAACACATACAAAAGAAACAGGACAGAAAACAGCCACC
5UTR-16 (50nt)
50nt unnatural 5′ UTR with GGG, kozak sequence (GCCACC) and minimal secondary
structure
(SEQ ID NO: 56)
GGGAACGACAAGAAACACATACAAAAGAAACAGGACAGAAAACAGCCACC
5UTR-17 (60nt)
60nt unnatural 5′ UTR with GGG, kozak sequence (GCCACC) and minimal secondary
structure
(SEQ ID NO: 57)
GGGCATAAACATAAACGACAAGAAACACATACAAAAGAAACAGGACAGAAAACAGCCACC
5UTR-18 (70nt = 0305K)
70nt unnatural 5′ UTR with GGG, kozak sequence (GCCACC) and minimal secondary
structure
(SEQ ID NO: 58)
GGGAAGAGATAAACATAAACATAAACGACAAGAAACACATACAAAAGAAACAGGACAGAAAACAGCCACC
5UTR-19 (100nt)
100nt unnatural 5′ UTR with GGG, kozak sequence (GCCACC) and minimal secondary
structure
(SEQ ID NO: 59)
GGGAACAACAGAGGAGAAGAGGGAACAGGACACAAGAGATAAACATAAACATAAACGACAAGAAACACATACAAAAGAAACAGG
ACAGAAAACAGCCACC
5UTR-20 (50nt = 0301K-1)
Alternative 50nt unnatural 5′ UTR with GGG, kozak sequence (GCCACC) and minimal
secondary structure
(SEQ ID NO: 60)
GGGAAAGAAAAAGATAAGGAGAAAAATAAAGAGAGGAAGAAAAAGCCACC
5UTR-21 (50nt = 0301K-2)
Alternative 50nt unnatural 5′ UTR with GGG, kozak sequence (GCCACC) and minimal
secondary structure
(SEQ ID NO: 61)
GGGAAAAGTAGAAAGAAAGAAAGAAGAGAAAATAAAGACAAAGAGCCACC
5UTR-22 (70nt = 1015K-A)
70nt unnatural 5′ UTR with G, kozak sequence (GCCACC), minimal secondary structure
and modified ACGU content (25% GC, 27% A, 37% U)
(SEQ ID NO: 62)
GCTTTCACTATTTCATTCATTTCATTCACACATTACACTTACATCACATCCACATTACATTTCTGCCACC
5UTR-23 (70nt = 1015K-B)
70nt unnatural 5′ UTR with G, kozak sequence (GCCACC), minimal secondary structure
and modified ACGU content (25% GC, 17% A, 48% U)
(SEQ ID NO: 63)
GCTTTCACTATTTCATTCATTTCATTCTCTCATTACTCTTACTTCTCTTCCTCATTACATTTCTGCCACC
3UTR-1 (T44/45)
3′ UTR from transcript ENSMUST00000102844 and ENSMUST00000102845 of mouse
ribosomal protein S27a gene (Gene symbol: RPS27A)
(SEQ ID NO: 64)
TTGTGTATGCGTTAATAAAAAGAAGGAACTCGTA
3UTR-2 (T35)
3′UTR from transcript EN5T00000404735 of human ribosomal protein 527a gene
(Gene symbol: RPS27A)
(SEQ ID NO: 65)
CTGTATGAGTTAATAAAAGACATGAACTAACATTTATTGTTGGGTTTTATTGCAGTAAAAAGAATGGTTTTTAAGCACCAAATT
GATGGTCACACCATTTCCTTTTAGTAGTGCTACTGCTATCGCTGTGTGAATGTTGCCTCTGGGGATTATGTGACCCAGTGGTTC
TGTATACCTG
3UTR-3 (T17)
3′UTR from transcript EN5T00000272317 of human ribosomal protein 527a gene
(Gene symbol: RPS27A)
(SEQ ID NO: 66)
CTGTATGAGTTAATAAAAGACATGAACTAACATTTATTGTTGGGTTTTATTGCAGTAAAAAGAATGGTTTTTAAGCACCAAATT
GATGGTCACACCATTTCCTTTTAGTAGTGCTACTGCTATCGCTGTGTGAATGTTGCCTCTGGGGATTATGTGACCCAGTGGTTC
TGTATACCTGCCAGGTGCCAACCACTTGTAAAGGTCTTGATATTTTCAATTCTTAGACTACCTATACTTTGGCAGAAGTTATAT
TTAATGTAAGTTGTCTAAATATAA
T44-TOP-uATG-Calnexin-EGFP (ER targeting eGFP mRNA)
(SEQ ID NO: 67)
GATCCGCCATCGTGGGTGAGTGTtagCTCTGTGGCCGCGCTCTGGCTAGTGGCGCTACGCGTCGCTCTCACGGGTGTCGTCGGA
TCTAATCCGTCTCTTTTCGAtagCAGGTGGAGCCGCCGCCACGatggaagggaagtggttgctgtgtatgttactggtgcttgg
aactgctattgttgaggctcatgatggacatgatgatgatgtgattgatattgaggatgaccttgacgatgtcattgaagaggt
agaagactcaaaaccagataccactgctcctccttcatctcccaaggttacttacaaagctccagttccaacaggggaagtata
ttttgctgattcttttgacagaggaactctgtcagggtggattttatccaaagccaagaaagacgataccgatgatgaaattgc
caaatatgatggaaagtgggaggtagaggaaatgaaggagtcaaagcttccaggtgataaaggacttgtgttgatgtctcgggc
caagcatcatgccatctctgctaaactgaacaagcccttcctgtttgacaccaagcctctcattgttcagtatgaggttaattt
ccaaaatggaatagaatgtggtggtgcctatgtgaaactgctttctaaaacaccagaactcaacctggatcagttccatgacaa
gaccccttatacgattatgtttggtccagataaatgtggagaggactataaactgcacttcatcttccgacacaaaaaccccaa
aacgggtatctatgaagaaaaacatgctaagaggccagatgcagatctgaagacctattttactgataagaaaacacatcttta
cacactaatcttgaatccagataatagttttgaaatactggttgaccaatctgtggtgaatagtggaaatctgctcaatgacat
gactcctcctgtaaatccttcacgtgaaattgaggacccagaagaccggaagcccgaggattgggatgaaagaccaaaaatccc
agatccagaagctgtcaagccagatgactgggatgaagatgcccctgctaagattccagatgaagaggccacaaaacccgaagg
ctggttagatgatgagcctgagtacgtacctgatccagacgcagagaaacctgaggattgggatgaagacatggatggagaatg
ggaggctcctcagattgccaaccctagatgtgagtcagctcctggatgtggtgtctggcagcgacctgtgattgacaaccccaa
ttataaaggcaaatggaagcctectatgattgacaatcccagttaccagggaatctggaaacccaggaaaataccaaatccaga
tttctttgaagatctggaacattcagaatgactcatttagtgctattggtttggagctgtggtccatgacctctgacatttttt
ttgacaactttatcatttgtgctgatcgaagaatagttgatgattgggccaatgatggatggggcctgaagaaagctgctgatg
gggctgctgagccaggcgttgtggggcagatgaacgaggcagctgaagagcgcccgtggctgtgggtagtctatattctaactg
tagcccttcctgtgttcctggttatcctcttctgctgttctggaaagaaacagaccagtggtatggagtataagaaaactgatg
cacctcaaccggatgtgaaggaagaggaagaagagaaggaagaggaaaaggacaagggagatgaggaggaggaaggagaagaga
aacttgaagagaaacagaaaagtgatgctgaagaagatggtggcactgtcagtcaagaggaggaagacagaaaacctaaagcag
aggaggatgaaattttgaacagatcaccaagaaacagaaagccacgaagagagCTCGAGGTGAGCAAGGGCGAGGAGCTGTTCA
CCGGGGTGGTGCCCATCCTGGTCGAGCTGGACGGCGACGTaAACGGCCACAAGTTCAGCGTGTCCGGCGAGGGCGAGGGCGATG
CCACCTACGGCAAGCTGACCCTGAAGTTCATCTGCACCACCGGCAAGCTGCCCGTGCCCTGGCCCACCCTCGTGACCACCCTGA
CCTACGGCGTGCAGTGCTTCAGCCGCTACCCCGACCACATGAAGCAGCACGACTTCTTCAAGTCCGCCATGCCCGAAGGCTACG
TCCAGGAGCGCACCATCTTCTTCAAGGACGACGGCAACTACAAGACCCGCGCCGAGGTGAAGTTCGAGGGCGACACCCTGGTGA
ACCGCATCGAGCTGAAGGGCATCGACTTCAAGGAGGACGGCAACATCCTGGGGCACAAGCTGGAGTACAACTACAACAGCCACA
ACGTCTATATCATGGCCGACAAGCAGAAGAACGGCATCAAGGTGAACTTCAAGATCCGCCACAACATCGAGGACGGCAGCGTGC
AGCTCGCCGACCACTACCAGCAGAACACCCCCATCGGCGACGGCCCCGTGCTGCTGCCCGACAACCACTACCTGAGCACCCAGT
CCGCCCTGAGCAAAGACCCCAACGAGAAGCGCGATCACATGGTCCTGCTGGAGTTCGTGACCGCCGCCGGGATCACTCtCGGCA
TGGACGAGCTGTACAAGTCTAGAtgaTTGTGTATGCGTTAATAAAAAGAAGGAACTCGTA
T44-TOP-uATG-Calnexin-mCherry (ER targeting mCherry mRNA)
(SEQ ID NO: 68)
GATCCGCCATCGTGGGTGAGTGTtagCTCTGTGGCCGCGCTCTGGCTAGTGGCGCTACGCGTCGCTCTCACGGGTGTCGTCGGA
TCTAATCCGTCTCTTTTCGAtagCAGGTGGAGCCGCCGCCACGatggaagggaagtggttgctgtgtatgttactggtgcttgg
aactgctattgttgaggctcatgatggacatgatgatgatgtgattgatattgaggatgaccttgacgatgtcattgaagaggt
agaagactcaaaaccagataccactgctcctccttcatctcccaaggttacttacaaagctccagttccaacaggggaagtata
ttttgctgattcttttgacagaggaactctgtcagggtggattttatccaaagccaagaaagacgataccgatgatgaaattgc
caaatatgatggaaagtgggaggtagaggaaatgaaggagtcaaagcttccaggtgataaaggacttgtgttgatgtctcgggc
caagcatcatgccatctctgctaaactgaacaagcccttcctgtttgacaccaagcctctcattgttcagtatgaggttaattt
ccaaaatggaatagaatgtggtggtgcctatgtgaaactgctttctaaaacaccagaactcaacctggatcagttccatgacaa
gaccccttatacgattatgtttggtccagataaatgtggagaggactataaactgcacttcatcttccgacacaaaaaccccaa
aacgggtatctatgaagaaaaacatgctaagaggccagatgcagatctgaagacctattttactgataagaaaacacatcttta
cacactaatcttgaatccagataatagttttgaaatactggttgaccaatctgtggtgaatagtggaaatctgctcaatgacat
gactcctcctgtaaatccttcacgtgaaattgaggacccagaagaccggaagcccgaggattgggatgaaagaccaaaaatccc
agatccagaagctgtcaagccagatgactgggatgaagatgcccctgctaagattccagatgaagaggccacaaaacccgaagg
ctggttagatgatgagcctgagtacgtacctgatccagacgcagagaaacctgaggattgggatgaagacatggatggagaatg
ggaggctcctcagattgccaaccctagatgtgagtcagctcctggatgtggtgtctggcagcgacctgtgattgacaaccccaa
ttataaaggcaaatggaagcctcctatgattgacaatcccagttaccagggaatctggaaacccaggaaaataccaaatccaga
tttctttgaagatctggaacattcagaatgactccttttagtgctattggtttggagctgtggtccatgacctctgacattttt
tttgacaactttatcatttgtgctgatcgaagaatagttgatgattgggccaatgatggatggggcctgaagaaagctgctgat
ggggctgctgagccaggcgttgtggggcagatgaacgaggcagctgaagagcgcccgtggctgtgggtagtctatattctaact
gtagcccttcctgtgttcctggttatcctcttctgctgttctggaaagaaacagaccagtggtatggagtataagaaaactgat
gcacctcaaccggatgtgaaggaagaggaagaagagaaggaagaggaaaaggacaagggagatgaggaggaggaaggagaagag
aaacttgaagagaaacagaaaagtgatgctgaagaagatggtggcactgtcagtcaagaggaggaagacagaaaacctaaagca
gaggaggatgaaattttgaacagatcaccaagaaacagaaagccacgaagagagCTCGAGGTGAGCAAGGGCGAGGAGGATAAC
ATGGCCATCATCAAGGAGTTCATGCGCTTCAAGGTGCACATGGAGGGCTCCGTGAACGGCCACGAGTTCGAGATCGAGGGCGAG
GGCGAGGGCCGCCCCTACGAGGGCACCCAGACCGCCAAGCTGAAGGTGACCAAGGGTGGCCCCCTGCCCTTCGCCTGGGACATC
CTGTCCCCTCAGTTCATGTACGGCTCCAAGGCCTACGTGAAGCACCCCGCCGACATCCCCGACTACTTGAAGCTGTCCTTCCCC
GAGGGCTTCAAGTGGGAGCGCGTGATGAACTTCGAGGACGGCGGCGTGGTGACCGTGACCCAGGACTCCTCCCTGCAGGACGGC
GAGTTCATCTACAAGGTGAAGCTGCGCGGCACCAACTTCCCCTCCGACGGCCCCGTAATGCAGAAGAAGACCATGGGCTGGGAG
GCCTCCTCCGAGCGGATGTACCCCGAGGACGGCGCCCTGAAGGGCGAGATCAAGCAGAGGCTGAAGCTGAAGGACGGCGGCCAC
TACGACGCTGAGGTCAAGACCACCTACAAGGCCAAGAAGCCCGTGCAGCTGCCCGGCGCCTACAACGTCAACATCAAGTTGGAC
ATCACCTCCCACAACGAGGACTACACCATCGTGGAACAGTACGAACGCGCCGAGGGCCGCCACTCCACCGGCGGCATGGACGAG
CTGTACAAGTCTAGAtgaTTGTGTATGCGTTAATAAAAAGAAGGAACTCGTA
T44-TOP-uATG-TOM20-EGFP (Mitochondria targeting eGFP mRNA)
(SEQ ID NO: 69)
GATCCGCCATCGTGGGTGAGTGTtagCTCTGTGGCCGCGCTCTGGCTAGTGGCGCTACGCGTCGCTCTCACGGGTGTCGTCGGA
TCTAATCCGTCTCTTTTCGAtagCAGGTGGAGCCGCCGCCACGAtggtgggacggaacagcgccatcgctgcaggagtgtgcgg
tgccctatcatagggtactgcatctactttgaccgcaaaaggaggagtgaccccaacCTCGAGGTGAGCAAGGGCGAGGAGCTG
TTCACCGGGGTGGTGCCCATCCTGGTCGAGCTGGACGGCGACGTaAACGGCCACAAGTTCAGCGTGTCCGGCGAGGGCGAGGGC
GATGCCACCTACGGCAAGCTGACCCTGAAGTTCATCTGCACCACCGGCAAGCTGCCCGTGCCCTGGCCCACCCTCGTGACCACC
CTGACCTACGGCGTGCAGTGCTTCAGCCGCTACCCCGACCACATGAAGCAGCACGACTTCTTCAAGTCCGCCATGCCCGAAGGC
TACGTCCAGGAGCGCACCATCTTCTTCAAGGACGACGGCAACTACAAGACCCGCGCCGAGGTGAAGTTCGAGGGCGACACCCTG
GTGAACCGCATCGAGCTGAAGGGCATCGACTTCAAGGAGGACGGCAACATCCTGGGGCACAAGCTGGAGTACAACTACAACAGC
CACAACGTCTATATCATGGCCGACAAGCAGAAGAACGGCATCAAGGTGAACTTCAAGATCCGCCACAACATCGAGGACGGCAGC
GTGCAGCTCGCCGACCACTACCAGCAGAACACCCCCATCGGCGACGGCCCCGTGCTGCTGCCCGACAACCACTACCTGAGCACC
CAGTCCGCCCTGAGCAAAGACCCCAACGAGAAGCGCGATCACATGGTCCTGCTGGAGTTCGTGACCGCCGCCGGGATCACTCtC
GGCATGGACGAGCTGTACAAGTCTAGAtgaTTGTGTATGCGTTAATAAAAAGAAGGAACTCGTA
T44-TOP-uATG-TOM20-mCherry (Mitochondria targeting mCherry mRNA)
(SEQ ID NO: 70)
GATCCGCCATCGTGGGTGAGTGTtagCTCTGTGGCCGCGCTCTGGCTAGTGGCGCTACGCGTCGCTCTCACGGGTGTCGTCGGA
TCTAATCCGTCTCTTTTCGAtagCAGGTGGAGCCGCCGCCACGAtggtgggacggaacagcgccatcgctgcaggagtgtgcgg
tgccctatcatagggtactgcatctactttgaccgcaaaaggaggagtgaccccaacCTCGAGGTGAGCAAGGGCGAGGAGGAT
AACATGGCCATCATCAAGGAGTTCATGCGCTTCAAGGTGCACATGGAGGGCTCCGTGAACGGCCACGAGTTCGAGATCGAGGGC
GAGGGCGAGGGCCGCCCCTACGAGGGCACCCAGACCGCCAAGCTGAAGGTGACCAAGGGTGGCCCCCTGCCCTTCGCCTGGGAC
ATCCTGTCCCCTCAGTTCATGTACGGCTCCAAGGCCTACGTGAAGCACCCCGCCGACATCCCCGACTACTTGAAGCTGTCCTTC
CCCGAGGGCTTCAAGTGGGAGCGCGTGATGAACTTCGAGGACGGCGGCGTGGTGACCGTGACCCAGGACTCCTCCCTGCAGGAC
GGCGAGTTCATCTACAAGGTGAAGCTGCGCGGCACCAACTTCCCCTCCGACGGCCCCGTAATGCAGAAGAAGACCATGGGCTGG
GAGGCCTCCTCCGAGCGGATGTACCCCGAGGACGGCGCCCTGAAGGGCGAGATCAAGCAGAGGCTGAAGCTGAAGGACGGCGGC
CACTACGACGCTGAGGTCAAGACCACCTACAAGGCCAAGAAGCCCGTGCAGCTGCCCGGCGCCTACAACGTCAACATCAAGTTG
GACATCACCTCCCACAACGAGGACTACACCATCGTGGAACAGTACGAACGCGCCGAGGGCCGCCACTCCACCGGCGGCATGGAC
GAGCTGTACAAGTCTAGAtgaTTGTGTATGCGTTAATAAAAAGAAGGAACTCGTA
T44-TOP-uATG-CatB-EGFP (Lysosome targeting eGFP mRNA)
(SEQ ID NO: 71)
GATCCGCCATCGTGGGTGAGTGTtagCTCTGTGGCCGCGCTCTGGCTAGTGGCGCTACGCGTCGCTCTCACGGGTGTCGTCGGA
TCTAATCCGTCTCTTTTCGAtagCAGGTGGAGCCGCCGCCACGATGTGGTGGTCCTTGATCCTTCTTTCTTGCCTGCTGGCACT
GACCAGTGCCCATGACAAGCCTTCCTTCCACCCGCTGTCGGATGACCTGATTAACTATATCAACAAACAGAATACAACATGGCA
GGCTGGACGCAACTTCTACAATGTTGACATAAGCTATCTGAAGAAGCTGTGTGGCACTGTCCTGGGTGGACCCAAACTGCCAGG
AAGGGTTGCGTTCGGTGAGGACATAGATCTACCTGAAACCTTTGATGCACGGGAACAATGGTCCAACTGCCCGACCATTGGACA
GATTAGAGACCAGGGCTCCTGCGGCTCTTGTTGGGCATTTGGGGCAGTGGAAGCCATTTCTGACCGAACCTGCATTCACACCAA
TGGCCGAGTCAACGTGGAGGTGTCTGCTGAAGACCTGCTTACTTGCTGTGGTATCCAGTGTGGGGACGGCTGTAATGGTGGCTA
TCCCTCTGGAGCATGGAGCTTCTGGACAAAAAAAGGCCTGGTTTCAGGTGGAGTCTACAATTCTCATGTAGGCTGCTTACCATA
CACCATCCCTCCCTGCGAGCACCATGTCAATGGCTCCCGTCCCCCATGCACTGGAGAAGGAGATACTCCCAGGTGCAACAAGAG
CTGTGAAGCTGGCTACTCCCCATCCTACAAAGAGGATAAGCACTTTGGGTACACTTCCTACAGCGTGTCTAACAGTGTGAAGGA
GATCATGGCAGAAATCTACAAAAATGGCCCAGTGGAGGGTGCCTTCACTGTGTTTTCTGACTTCTTGACTTACAAATCAGGAGT
ATACAAGCATGAAGCCGGTGATATGATGGGTGGCCACGCCATCCGCATCCTGGGCTGGGGAGTAGAGAATGGAGTTCCCTACTG
GCTGGCAGCCAACTCTTGGAACCTTGACTGGGGTGATAATGGCTTCTTTAAAATCCTCAGAGGAGAAAACCACTGTGGCATTGA
ATCAGAAATTGTGGCTGGAATCCCACGCACTGACCAGTACTGGGGAAGATTCGTGAGCAAGGGCGAGGAGCTGTTCACCGGGGT
GGTGCCCATCCTGGTCGAGCTGGACGGCGACGTaAACGGCCACAAGTTCAGCGTGTCCGGCGAGGGCGAGGGCGATGCCACCTA
CGGCAAGCTGACCCTGAAGTTCATCTGCACCACCGGCAAGCTGCCCGTGCCCTGGCCCACCCTCGTGACCACCCTGACCTACGG
CGTGCAGTGCTTCAGCCGCTACCCCGACCACATGAAGCAGCACGACTTCTTCAAGTCCGCCATGCCCGAAGGCTACGTCCAGGA
GCGCACCATCTTCTTCAAGGACGACGGCAACTACAAGACCCGCGCCGAGGTGAAGTTCGAGGGCGACACCCTGGTGAACCGCAT
CGAGCTGAAGGGCATCGACTTCAAGGAGGACGGCAACATCCTGGGGCACAAGCTGGAGTACAACTACAACAGCCACAACGTCTA
TATCATGGCCGACAAGCAGAAGAACGGCATCAAGGTGAACTTCAAGATCCGCCACAACATCGAGGACGGCAGCGTGCAGCTCGC
CGACCACTACCAGCAGAACACCCCCATCGGCGACGGCCCCGTGCTGCTGCCCGACAACCACTACCTGAGCACCCAGTCCGCCCT
GAGCAAAGACCCCAACGAGAAGCGCGATCACATGGTCCTGCTGGAGTTCGTGACCGCCGCCGGGATCACTCtCGGCATGGACGA
GCTGTACAAGtgaTTGTGTATGCGTTAATAAAAAGAAGGAACTCGTA
T44-TOP-uATG-CatB-mCherry (Lysosome targeting mCherry mRNA)
(SEQ ID NO: 72)
GATCCGCCATCGTGGGTGAGTGTtagCTCTGTGGCCGCGCTCTGGCTAGTGGCGCTACGCGTCGCTCTCACGGGTGTCGTCGGA
TCTAATCCGTCTCTTTTCGAtagCAGGTGGAGCCGCCGCCACGATGTGGTGGTCCTTGATCCTTCTTTCTTGCCTGCTGGCACT
GACCAGTGCCCATGACAAGCCTTCCTTCCACCCGCTGTCGGATGACCTGATTAACTATATCAACAAACAGAATACAACATGGCA
GGCTGGACGCAACTTCTACAATGTTGACATAAGCTATCTGAAGAAGCTGTGTGGCACTGTCCTGGGTGGACCCAAACTGCCAGG
AAGGGTTGCGTTCGGTGAGGACATAGATCTACCTGAAACCTTTGATGCACGGGAACAATGGTCCAACTGCCCGACCATTGGACA
GATTAGAGACCAGGGCTCCTGCGGCTCTTGTTGGGCATTTGGGGCAGTGGAAGCCATTTCTGACCGAACCTGCATTCACACCAA
TGGCCGAGTCAACGTGGAGGTGTCTGCTGAAGACCTGCTTACTTGCTGTGGTATCCAGTGTGGGGACGGCTGTAATGGTGGCTA
TCCCTCTGGAGCATGGAGCTTCTGGACAAAAAAAGGCCTGGTTTCAGGTGGAGTCTACAATTCTCATGTAGGCTGCTTACCATA
CACCATCCCTCCCTGCGAGCACCATGTCAATGGCTCCCGTCCCCCATGCACTGGAGAAGGAGATACTCCCAGGTGCAACAAGAG
CTGTGAAGCTGGCTACTCCCCATCCTACAAAGAGGATAAGCACTTTGGGTACACTTCCTACAGCGTGTCTAACAGTGTGAAGGA
GATCATGGCAGAAATCTACAAAAATGGCCCAGTGGAGGGTGCCTTCACTGTGTTTTCTGACTTCTTGACTTACAAATCAGGAGT
ATACAAGCATGAAGCCGGTGATATGATGGGTGGCCACGCCATCCGCATCCTGGGCTGGGGAGTAGAGAATGGAGTTCCCTACTG
GCTGGCAGCCAACTCTTGGAACCTTGACTGGGGTGATAATGGCTTCTTTAAAATCCTCAGAGGAGAAAACCACTGTGGCATTGA
ATCAGAAATTGTGGCTGGAATCCCACGCACTGACCAGTACTGGGGAAGATTCGTGAGCAAGGGCGAGGAGGATAACATGGCCAT
CATCAAGGAGTTCATGCGCTTCAAGGTGCACATGGAGGGCTCCGTGAACGGCCACGAGTTCGAGATCGAGGGCGAGGGCGAGGG
CCGCCCCTACGAGGGCACCCAGACCGCCAAGCTGAAGGTGACCAAGGGTGGCCCCCTGCCCTTCGCCTGGGACATCCTGTCCCC
TCAGTTCATGTACGGCTCCAAGGCCTACGTGAAGCACCCCGCCGACATCCCCGACTACTTGAAGCTGTCCTTCCCCGAGGGCTT
CAAGTGGGAGCGCGTGATGAACTTCGAGGACGGCGGCGTGGTGACCGTGACCCAGGACTCCTCCCTGCAGGACGGCGAGTTCAT
CTACAAGGTGAAGCTGCGCGGCACCAACTTCCCCTCCGACGGCCCCGTAATGCAGAAGAAGACCATGGGCTGGGAGGCCTCCTC
CGAGCGGATGTACCCCGAGGACGGCGCCCTGAAGGGCGAGATCAAGCAGAGGCTGAAGCTGAAGGACGGCGGCCACTACGACGC
TGAGGTCAAGACCACCTACAAGGCCAAGAAGCCCGTGCAGCTGCCCGGCGCCTACAACGTCAACATCAAGTTGGACATCACCTC
CCACAACGAGGACTACACCATCGTGGAACAGTACGAACGCGCCGAGGGCCGCCACTCCACCGGCGGCATGGACGAGCTGTACAA
GtgaTTGTGTATGCGTTAATAAAAAGAAGGAACTCGTA
T44-top-uATG-NLS-eGFP-NLS (Nucleus targeting eGFP mRNA)
(SEQ ID NO: 73)
GATCCGCCATCGTGGGTGAGTGTtagCTCTGTGGCCGCGCTCTGGCTAGTGGCGCTACGCGTCGCTCTCACGGGTGTCGTCGGA
TCTAATCCGTCTCTTTTCGAtagCAGGTGGAGCCGCCGCCACGATGGCCCCAAAGAAGAAGCGGAAGGTCGGTATCCACGGAGT
CCCAGCAGCCGTGAGCAAGGGCGAGGAGCTGTTCACCGGGGTGGTGCCCATCCTGGTCGAGCTGGACGGCGACGTaAACGGCCA
CAAGTTCAGCGTGTCCGGCGAGGGCGAGGGCGATGCCACCTACGGCAAGCTGACCCTGAAGTTCATCTGCACCACCGGCAAGCT
GCCCGTGCCCTGGCCCACCCTCGTGACCACCCTGACCTACGGCGTGCAGTGCTTCAGCCGCTACCCCGACCACATGAAGCAGCA
CGACTTCTTCAAGTCCGCCATGCCCGAAGGCTACGTCCAGGAGCGCACCATCTTCTTCAAGGACGACGGCAACTACAAGACCCG
CGCCGAGGTGAAGTTCGAGGGCGACACCCTGGTGAACCGCATCGAGCTGAAGGGCATCGACTTCAAGGAGGACGGCAACATCCT
GGGGCACAAGCTGGAGTACAACTACAACAGCCACAACGTCTATATCATGGCCGACAAGCAGAAGAACGGCATCAAGGTGAACTT
CAAGATCCGCCACAACATCGAGGACGGCAGCGTGCAGCTCGCCGACCACTACCAGCAGAACACCCCCATCGGCGACGGCCCCGT
GCTGCTGCCCGACAACCACTACCTGAGCACCCAGTCCGCCCTGAGCAAAGACCCCAACGAGAAGCGCGATCACATGGTCCTGCT
GGAGTTCGTGACCGCCGCCGGGATCACTCtCGGCATGGACGAGCTGTACAAGAAGCGTCCTGCTGCTACTAAGAAAGCTGGTCA
AGCTAAGAAAAAGAAATAAGCGGCCGCTTGTGTATGCGTTAATAAAAAGAAGGAACTCGTA
T44-top-uATG-NLS-mCherry-NLS (Nucleus targeting mCherry mRNA)
(SEQ ID NO: 74)
GGGGATCCGCCATCGTGGGTGAGTGTtagCTCTGTGGCCGCGCTCTGGCTAGTGGCGCTACGCGTCGCTCTCACGGGTGTCGTC
GGATCTAATCCGTCTCTTTTCGAtagCAGGTGGAGCCGCCGCCACGATGGCCCCAAAGAAGAAGCGGAAGGTCGGTATCCACGG
AGTCCCAGCAGCCGTGAGCAAGGGCGAGGAGGATAACATGGCCATCATCAAGGAGTTCATGCGCTTCAAGGTGCACATGGAGGG
CTCCGTGAACGGCCACGAGTTCGAGATCGAGGGCGAGGGCGAGGGCCGCCCCTACGAGGGCACCCAGACCGCCAAGCTGAAGGT
GACCAAGGGTGGCCCCCTGCCCTTCGCCTGGGACATCCTGTCCCCTCAGTTCATGTACGGCTCCAAGGCCTACGTGAAGCACCC
CGCCGACATCCCCGACTACTTGAAGCTGTCCTTCCCCGAGGGCTTCAAGTGGGAGCGCGTGATGAACTTCGAGGACGGCGGCGT
GGTGACCGTGACCCAGGACTCCTCCCTGCAGGACGGCGAGTTCATCTACAAGGTGAAGCTGCGCGGCACCAACTTCCCCTCCGA
CGGCCCCGTAATGCAGAAGAAGACCATGGGCTGGGAGGCCTCCTCCGAGCGGATGTACCCCGAGGACGGCGCCCTGAAGGGCGA
GATCAAGCAGAGGCTGAAGCTGAAGGACGGCGGCCACTACGACGCTGAGGTCAAGACCACCTACAAGGCCAAGAAGCCCGTGCA
GCTGCCCGGCGCCTACAACGTCAACATCAAGTTGGACATCACCTCCCACAACGAGGACTACACCATCGTGGAACAGTACGAACG
CGCCGAGGGCCGCCACTCCACCGGCGGCATGGACGAGCTGTACAAGAAGCGTCCTGCTGCTACTAAGAAAGCTGGTCAAGCTAA
GAAAAAGAAATAAGCGGCCGCTTGTGTATGCGTTAATAAAAAGAAGGAACTCGTA
T44-TOP-uATG-TOM20-mCherry-P2A-Calnexin-eGFP
(SEQ ID NO: 75)
GATCCGCCATCGTGGGTGAGTGTtagCTCTGTGGCCGCGCTCTGGCTAGTGGCGCTACGCGTCGCTCTCACGGGTGTCGTCGGA
TCTAATCCGTCTCTTTTCGAtagCAGGTGGAGCCGCCGCCACGAtggtgggacggaacagcgccatcgctgcaggagtgtgcgg
tgccctatcatagggtactgcatctactttgaccgcaaaaggaggagtgaccccaacCTCGAGGTGAGCAAGGGCGAGGAGGAT
AACATGGCCATCATCAAGGAGTTCATGCGCTTCAAGGTGCACATGGAGGGCTCCGTGAACGGCCACGAGTTCGAGATCGAGGGC
GAGGGCGAGGGCCGCCCCTACGAGGGCACCCAGACCGCCAAGCTGAAGGTGACCAAGGGTGGCCCCCTGCCCTTCGCCTGGGAC
ATCCTGTCCCCTCAGTTCATGTACGGCTCCAAGGCCTACGTGAAGCACCCCGCCGACATCCCCGACTACTTGAAGCTGTCCTTC
CCCGAGGGCTTCAAGTGGGAGCGCGTGATGAACTTCGAGGACGGCGGCGTGGTGACCGTGACCCAGGACTCCTCCCTGCAGGAC
GGCGAGTTCATCTACAAGGTGAAGCTGCGCGGCACCAACTTCCCCTCCGACGGCCCCGTAATGCAGAAGAAGACCATGGGCTGG
GAGGCCTCCTCCGAGCGGATGTACCCCGAGGACGGCGCCCTGAAGGGCGAGATCAAGCAGAGGCTGAAGCTGAAGGACGGCGGC
CACTACGACGCTGAGGTCAAGACCACCTACAAGGCCAAGAAGCCCGTGCAGCTGCCCGGCGCCTACAACGTCAACATCAAGTTG
GACATCACCTCCCACAACGAGGACTACACCATCGTGGAACAGTACGAACGCGCCGAGGGCCGCCACTCCACCGGCGGCATGGAC
GAGCTGTACAAGggatccggcgcaacaaacttactctgctgaaacaagccggagatgtegaagagaatectggaccgATGGAAG
GGAAGTGGTTGCTGTGTATGTTACTGGTGCTTGGAACTGCTATTGTTGAGGCTCATGATGGACATGATGATGATGTGATTGATA
TTGAGGATGACCTTGACGATGTCATTGAAGAGGTAGAAGACTCAAAACCAGATACCACTGCTCCTCCTTCATCTCCCAAGGTTA
CTTACAAAGCTCCAGTTCCAACAGGGGAAGTATATTTTGCTGATTCTTTTGACAGAGGAACTCTGTCAGGGTGGATTTTATCCA
AAGCCAAGAAAGACGATACCGATGATGAAATTGCCAAATATGATGGAAAGTGGGAGGTAGAGGAAATGAAGGAGTCAAAGCTTC
CAGGTGATAAAGGACTTGTGTTGATGTCTCGGGCCAAGCATCATGCCATCTCTGCTAAACTGAACAAGCCCTTCCTGTTTGACA
CCAAGCCTCTCATTGTTCAGTATGAGGTTAATTTCCAAAATGGAATAGAATGTGGTGGTGCCTATGTGAAACTGCTTTCTAAAA
CACCAGAACTCAACCTGGATCAGTTCCATGACAAGACCCCTTATACGATTATGTTTGGTCCAGATAAATGTGGAGAGGACTATA
AACTGCACTTCATCTTCCGACACAAAAACCCCAAAACGGGTATCTATGAAGAAAAACATGCTAAGAGGCCAGATGCAGATCTGA
AGACCTATTTTACTGATAAGAAAACACATCTTTACACACTAATCTTGAATCCAGATAATAGTTTTGAAATACTGGTTGACCAAT
CTGTGGTGAATAGTGGAAATCTGCTCAATGACATGACTCCTCCTGTAAATCCTTCACGTGAAATTGAGGACCCAGAAGACCGGA
AGCCCGAGGATTGGGATGAAAGACCAAAAATCCCAGATCCAGAAGCTGTCAAGCCAGATGACTGGGATGAAGATGCCCCTGCTA
AGATTCCAGATGAAGAGGCCACAAAACCCGAAGGCTGGTTAGATGATGAGCCTGAGTACGTACCTGATCCAGACGCAGAGAAAC
CTGAGGATTGGGATGAAGACATGGATGGAGAATGGGAGGCTCCTCAGATTGCCAACCCTAGATGTGAGTCAGCTCCTGGATGTG
GTGTCTGGCAGCGACCTGTGATTGACAACCCCAATTATAAAGGCAAATGGAAGCCTCCTATGATTGACAATCCCAGTTACCAGG
GAATCTGGAAACCCAGGAAAATACCAAATCCAGATTTCTTTGAAGATCTGGAACCTTTCAGAATGACTCCTTTTAGTGCTATTG
GTTTGGAGCTGTGGTCCATGACCTCTGACATTTTTTTTGACAACTTTATCATTTGTGCTGATCGAAGAATAGTTGATGATTGGG
CCAATGATGGATGGGGCCTGAAGAAAGCTGCTGATGGGGCTGCTGAGCCAGGCGTTGTGGGGCAGATGAACGAGGCAGCTGAAG
AGCGCCCGTGGCTGTGGGTAGTCTATATTCTAACTGTAGCCCTTCCTGTGTTCCTGGTTATCCTCTTCTGCTGTTCTGGAAAGA
AACAGACCAGTGGTATGGAGTATAAGAAAACTGATGCACCTCAACCGGATGTGAAGGAAGAGGAAGAAGAGAAGGAAGAGGAAA
AGGACAAGGGAGATGAGGAGGAGGAAGGAGAAGAGAAACTTGAAGAGAAACAGAAAAGTGATGCTGAAGAAGATGGTGGCACTG
TCAGTCAAGAGGAGGAAGACAGAAAACCTAAAGCAGAGGAGGATGAAATTTTGAACAGATCACCAAGAAACAGAAAGCCACGAA
GAGAGGTGAGCAAGGGCGAGGAGCTGTTCACCGGGGTGGTGCCCATCCTGGTCGAGCTGGACGGCGACGTaAACGGCCACAAGT
TCAGCGTGTCCGGCGAGGGCGAGGGCGATGCCACCTACGGCAAGCTGACCCTGAAGTTCATCTGCACCACCGGCAAGCTGCCCG
TGCCCTGGCCCACCCTCGTGACCACCCTGACCTACGGCGTGCAGTGCTTCAGCCGCTACCCCGACCACATGAAGCAGCACGACT
TCTTCAAGTCCGCCATGCCCGAAGGCTACGTCCAGGAGCGCACCATCTTCTTCAAGGACGACGGCAACTACAAGACCCGCGCCG
AGGTGAAGTTCGAGGGCGACACCCTGGTGAACCGCATCGAGCTGAAGGGCATCGACTTCAAGGAGGACGGCAACATCCTGGGGC
ACAAGCTGGAGTACAACTACAACAGCCACAACGTCTATATCATGGCCGACAAGCAGAAGAACGGCATCAAGGTGAACTTCAAGA
TCCGCCACAACATCGAGGACGGCAGCGTGCAGCTCGCCGACCACTACCAGCAGAACACCCCCATCGGCGACGGCCCCGTGCTGC
TGCCCGACAACCACTACCTGAGCACCCAGTCCGCCCTGAGCAAAGACCCCAACGAGAAGCGCGATCACATGGTCCTGCTGGAGT
TCGTGACCGCCGCCGGGATCACTCtCGGCATGGACGAGCTGTACAAGtgaTTGTGTATGCGTTAATAAAAAGAAGGAACTCGTA
T44-TOP-uATG-TOM20-mCherry-P2A-CatB-eGFP
(SEQ ID NO: 76)
GATCCGCCATCGTGGGTGAGTGTtagCTCTGTGGCCGCGCTCTGGCTAGTGGCGCTACGCGTCGCTCTCACGGGTGTCGTCGGA
TCTAATCCGTCTCTTTTCGAtagCAGGTGGAGCCGCCGCCACGAtggtgggacggaacagcgccatcgctgcaggagtgtgcgg
tgccacttcatagggtactgcatctactttgaccgcaaaaggaggagtgaccccaacCTCGAGGTGAGCAAGGGCGAGGAGGAT
AACATGGCCATCATCAAGGAGTTCATGCGCTTCAAGGTGCACATGGAGGGCTCCGTGAACGGCCACGAGTTCGAGATCGAGGGC
GAGGGCGAGGGCCGCCCCTACGAGGGCACCCAGACCGCCAAGCTGAAGGTGACCAAGGGTGGCCCCCTGCCCTTCGCCTGGGAC
ATCCTGTCCCCTCAGTTCATGTACGGCTCCAAGGCCTACGTGAAGCACCCCGCCGACATCCCCGACTACTTGAAGCTGTCCTTC
CCCGAGGGCTTCAAGTGGGAGCGCGTGATGAACTTCGAGGACGGCGGCGTGGTGACCGTGACCCAGGACTCCTCCCTGCAGGAC
GGCGAGTTCATCTACAAGGTGAAGCTGCGCGGCACCAACTTCCCCTCCGACGGCCCCGTAATGCAGAAGAAGACCATGGGCTGG
GAGGCCTCCTCCGAGCGGATGTACCCCGAGGACGGCGCCCTGAAGGGCGAGATCAAGCAGAGGCTGAAGCTGAAGGACGGCGGC
CACTACGACGCTGAGGTCAAGACCACCTACAAGGCCAAGAAGCCCGTGCAGCTGCCCGGCGCCTACAACGTCAACATCAAGTTG
GACATCACCTCCCACAACGAGGACTACACCATCGTGGAACAGTACGAACGCGCCGAGGGCCGCCACTCCACCGGCGGCATGGAC
GAGCTGTACAAGggatccggcgcaacaaacttactctgctgaaacaagccggagatgtegaagagaatectggaccgATGTGGT
GGTCCTTGATCCTTCTTTCTTGCCTGCTGGCACTGACCAGTGCCCATGACAAGCCTTCCTTCCACCCGCTGTCGGATGACCTGA
TTAACTATATCAACAAACAGAATACAACATGGCAGGCTGGACGCAACTTCTACAATGTTGACATAAGCTATCTGAAGAAGCTGT
GTGGCACTGTCCTGGGTGGACCCAAACTGCCAGGAAGGGTTGCGTTCGGTGAGGACATAGATCTACCTGAAACCTTTGATGCAC
GGGAACAATGGTCCAACTGCCCGACCATTGGACAGATTAGAGACCAGGGCTCCTGCGGCTCTTGTTGGGCATTTGGGGCAGTGG
AAGCCATTTCTGACCGAACCTGCATTCACACCAATGGCCGAGTCAACGTGGAGGTGTCTGCTGAAGACCTGCTTACTTGCTGTG
GTATCCAGTGTGGGGACGGCTGTAATGGTGGCTATCCCTCTGGAGCATGGAGCTTCTGGACAAAAAAAGGCCTGGTTTCAGGTG
GAGTCTACAATTCTCATGTAGGCTGCTTACCATACACCATCCCTCCCTGCGAGCACCATGTCAATGGCTCCCGTCCCCCATGCA
CTGGAGAAGGAGATACTCCCAGGTGCAACAAGAGCTGTGAAGCTGGCTACTCCCCATCCTACAAAGAGGATAAGCACTTTGGGT
ACACTTCCTACAGCGTGTCTAACAGTGTGAAGGAGATCATGGCAGAAATCTACAAAAATGGCCCAGTGGAGGGTGCCTTCACTG
TGTTTTCTGACTTCTTGACTTACAAATCAGGAGTATACAAGCATGAAGCCGGTGATATGATGGGTGGCCACGCCATCCGCATCC
TGGGCTGGGGAGTAGAGAATGGAGTTCCCTACTGGCTGGCAGCCAACTCTTGGAACCTTGACTGGGGTGATAATGGCTTCTTTA
AAATCCTCAGAGGAGAAAACCACTGTGGCATTGAATCAGAAATTGTGGCTGGAATCCCACGCACTGACCAGTACTGGGGAAGAT
TCGTGAGCAAGGGCGAGGAGCTGTTCACCGGGGTGGTGCCCATCCTGGTCGAGCTGGACGGCGACGTaAACGGCCACAAGTTCA
GCGTGTCCGGCGAGGGCGAGGGCGATGCCACCTACGGCAAGCTGACCCTGAAGTTCATCTGCACCACCGGCAAGCTGCCCGTGC
CCTGGCCCACCCTCGTGACCACCCTGACCTACGGCGTGCAGTGCTTCAGCCGCTACCCCGACCACATGAAGCAGCACGACTTCT
TCAAGTCCGCCATGCCCGAAGGCTACGTCCAGGAGCGCACCATCTTCTTCAAGGACGACGGCAACTACAAGACCCGCGCCGAGG
TGAAGTTCGAGGGCGACACCCTGGTGAACCGCATCGAGCTGAAGGGCATCGACTTCAAGGAGGACGGCAACATCCTGGGGCACA
AGCTGGAGTACAACTACAACAGCCACAACGTCTATATCATGGCCGACAAGCAGAAGAACGGCATCAAGGTGAACTTCAAGATCC
GCCACAACATCGAGGACGGCAGCGTGCAGCTCGCCGACCACTACCAGCAGAACACCCCCATCGGCGACGGCCCCGTGCTGCTGC
CCGACAACCACTACCTGAGCACCCAGTCCGCCCTGAGCAAAGACCCCAACGAGAAGCGCGATCACATGGTCCTGCTGGAGTTCG
TGACCGCCGCCGGGATCACTCtCGGCATGGACGAGCTGTACAAGtgaTTGTGTATGCGTTAATAAAAAGAAGGAACTCGTA
T44-TOP-uATG-TOM20-mCherry-P2A-NLS-eGFP-NLS
(SEQ ID NO: 77)
GATCCGCCATCGTGGGTGAGTGTtagCTCTGTGGCCGCGCTCTGGCTAGTGGCGCTACGCGTCGCTCTCACGGGTGTCGTCGGA
TCTAATCCGTCTCTTTTCGAtagCAGGTGGAGCCGCCGCCACGAtggtgggacggaacagcgccatcgctgcaggagtgtgcgg
tgccacttcatagggtactgcatctactttgaccgcaaaaggaggagtgaccccaacCTCGAGGTGAGCAAGGGCGAGGAGGAT
AACATGGCCATCATCAAGGAGTTCATGCGCTTCAAGGTGCACATGGAGGGCTCCGTGAACGGCCACGAGTTCGAGATCGAGGGC
GAGGGCGAGGGCCGCCCCTACGAGGGCACCCAGACCGCCAAGCTGAAGGTGACCAAGGGTGGCCCCCTGCCCTTCGCCTGGGAC
ATCCTGTCCCCTCAGTTCATGTACGGCTCCAAGGCCTACGTGAAGCACCCCGCCGACATCCCCGACTACTTGAAGCTGTCCTTC
CCCGAGGGCTTCAAGTGGGAGCGCGTGATGAACTTCGAGGACGGCGGCGTGGTGACCGTGACCCAGGACTCCTCCCTGCAGGAC
GGCGAGTTCATCTACAAGGTGAAGCTGCGCGGCACCAACTTCCCCTCCGACGGCCCCGTAATGCAGAAGAAGACCATGGGCTGG
GAGGCCTCCTCCGAGCGGATGTACCCCGAGGACGGCGCCCTGAAGGGCGAGATCAAGCAGAGGCTGAAGCTGAAGGACGGCGGC
CACTACGACGCTGAGGTCAAGACCACCTACAAGGCCAAGAAGCCCGTGCAGCTGCCCGGCGCCTACAACGTCAACATCAAGTTG
GACATCACCTCCCACAACGAGGACTACACCATCGTGGAACAGTACGAACGCGCCGAGGGCCGCCACTCCACCGGCGGCATGGAC
GAGCTGTACAAGggatccggcgcaacaaacttctactgctgaaacaagccggagatgtegaagagaatectggaccgATGGCCC
CAAAGAAGAAGCGGAAGGTCGGTATCCACGGAGTCCCAGCAGCCGTGAGCAAGGGCGAGGAGCTGTTCACCGGGGTGGTGCCCA
TCCTGGTCGAGCTGGACGGCGACGTaAACGGCCACAAGTTCAGCGTGTCCGGCGAGGGCGAGGGCGATGCCACCTACGGCAAGC
TGACCCTGAAGTTCATCTGCACCACCGGCAAGCTGCCCGTGCCCTGGCCCACCCTCGTGACCACCCTGACCTACGGCGTGCAGT
GCTTCAGCCGCTACCCCGACCACATGAAGCAGCACGACTTCTTCAAGTCCGCCATGCCCGAAGGCTACGTCCAGGAGCGCACCA
TCTTCTTCAAGGACGACGGCAACTACAAGACCCGCGCCGAGGTGAAGTTCGAGGGCGACACCCTGGTGAACCGCATCGAGCTGA
AGGGCATCGACTTCAAGGAGGACGGCAACATCCTGGGGCACAAGCTGGAGTACAACTACAACAGCCACAACGTCTATATCATGG
CCGACAAGCAGAAGAACGGCATCAAGGTGAACTTCAAGATCCGCCACAACATCGAGGACGGCAGCGTGCAGCTCGCCGACCACT
ACCAGCAGAACACCCCCATCGGCGACGGCCCCGTGCTGCTGCCCGACAACCACTACCTGAGCACCCAGTCCGCCCTGAGCAAAG
ACCCCAACGAGAAGCGCGATCACATGGTCCTGCTGGAGTTCGTGACCGCCGCCGGGATCACTCtCGGCATGGACGAGCTGTACA
AGAAGCGTCCTGCTGCTACTAAGAAAGCTGGTCAAGCTAAGAAAAAGAAATAAGCGGCCGCTTGTGTATGCGTTAATAAAAAGA
AGGAACTCGTA
T44-TOP-uATG-TOM20-mCherry-GGGGS4-Calexin-eGFP
(SEQ ID NO: 78)
GATCCGCCATCGTGGGTGAGTGTtagCTCTGTGGCCGCGCTCTGGCTAGTGGCGCTACGCGTCGCTCTCACGGGTGTCGTCGGA
TCTAATCCGTCTCTTTTCGAtagCAGGTGGAGCCGCCGCCACGAtggtgggacggaacagcgccatcgctgcaggagtgtgcgg
tgccacttcatagggtactgcatctactttgaccgcaaaaggaggagtgaccccaacCTCGAGGTGAGCAAGGGCGAGGAGGAT
AACATGGCCATCATCAAGGAGTTCATGCGCTTCAAGGTGCACATGGAGGGCTCCGTGAACGGCCACGAGTTCGAGATCGAGGGC
GAGGGCGAGGGCCGCCCCTACGAGGGCACCCAGACCGCCAAGCTGAAGGTGACCAAGGGTGGCCCCCTGCCCTTCGCCTGGGAC
ATCCTGTCCCCTCAGTTCATGTACGGCTCCAAGGCCTACGTGAAGCACCCCGCCGACATCCCCGACTACTTGAAGCTGTCCTTC
CCCGAGGGCTTCAAGTGGGAGCGCGTGATGAACTTCGAGGACGGCGGCGTGGTGACCGTGACCCAGGACTCCTCCCTGCAGGAC
GGCGAGTTCATCTACAAGGTGAAGCTGCGCGGCACCAACTTCCCCTCCGACGGCCCCGTAATGCAGAAGAAGACCATGGGCTGG
GAGGCCTCCTCCGAGCGGATGTACCCCGAGGACGGCGCCCTGAAGGGCGAGATCAAGCAGAGGCTGAAGCTGAAGGACGGCGGC
CACTACGACGCTGAGGTCAAGACCACCTACAAGGCCAAGAAGCCCGTGCAGCTGCCCGGCGCCTACAACGTCAACATCAAGTTG
GACATCACCTCCCACAACGAGGACTACACCATCGTGGAACAGTACGAACGCGCCGAGGGCCGCCACTCCACCGGCGGCATGGAC
GAGCTGTACAAGGGAGGTGGAGGCAGCGGAGGCGGGGGCAGTGGAGGAGGGGGTTCCGGTGGTGGTGGTAGTATGGAAGGGAAG
TGGTTGCTGTGTATGTTACTGGTGCTTGGAACTGCTATTGTTGAGGCTCATGATGGACATGATGATGATGTGATTGATATTGAG
GATGACCTTGACGATGTCATTGAAGAGGTAGAAGACTCAAAACCAGATACCACTGCTCCTCCTTCATCTCCCAAGGTTACTTAC
AAAGCTCCAGTTCCAACAGGGGAAGTATATTTTGCTGATTCTTTTGACAGAGGAACTCTGTCAGGGTGGATTTTATCCAAAGCC
AAGAAAGACGATACCGATGATGAAATTGCCAAATATGATGGAAAGTGGGAGGTAGAGGAAATGAAGGAGTCAAAGCTTCCAGGT
GATAAAGGACTTGTGTTGATGTCTCGGGCCAAGCATCATGCCATCTCTGCTAAACTGAACAAGCCCTTCCTGTTTGACACCAAG
CCTCTCATTGTTCAGTATGAGGTTAATTTCCAAAATGGAATAGAATGTGGTGGTGCCTATGTGAAACTGCTTTCTAAAACACCA
GAACTCAACCTGGATCAGTTCCATGACAAGACCCCTTATACGATTATGTTTGGTCCAGATAAATGTGGAGAGGACTATAAACTG
CACTTCATCTTCCGACACAAAAACCCCAAAACGGGTATCTATGAAGAAAAACATGCTAAGAGGCCAGATGCAGATCTGAAGACC
TATTTTACTGATAAGAAAACACATCTTTACACACTAATCTTGAATCCAGATAATAGTTTTGAAATACTGGTTGACCAATCTGTG
GTGAATAGTGGAAATCTGCTCAATGACATGACTCCTCCTGTAAATCCTTCACGTGAAATTGAGGACCCAGAAGACCGGAAGCCC
GAGGATTGGGATGAAAGACCAAAAATCCCAGATCCAGAAGCTGTCAAGCCAGATGACTGGGATGAAGATGCCCCTGCTAAGATT
CCAGATGAAGAGGCCACAAAACCCGAAGGCTGGTTAGATGATGAGCCTGAGTACGTACCTGATCCAGACGCAGAGAAACCTGAG
GATTGGGATGAAGACATGGATGGAGAATGGGAGGCTCCTCAGATTGCCAACCCTAGATGTGAGTCAGCTCCTGGATGTGGTGTC
TGGCAGCGACCTGTGATTGACAACCCCAATTATAAAGGCAAATGGAAGCCTCCTATGATTGACAATCCCAGTTACCAGGGAATC
TGGAAACCCAGGAAAATACCAAATCCAGATTTCTTTGAAGATCTGGAACCTTTCAGAATGACTCCTTTTAGTGCTATTGGTTTG
GAGCTGTGGTCCATGACCTCTGACATTTTTTTTGACAACTTTATCATTTGTGCTGATCGAAGAATAGTTGATGATTGGGCCAAT
GATGGATGGGGCCTGAAGAAAGCTGCTGATGGGGCTGCTGAGCCAGGCGTTGTGGGGCAGATGAACGAGGCAGCTGAAGAGCGC
CCGTGGCTGTGGGTAGTCTATATTCTAACTGTAGCCCTTCCTGTGTTCCTGGTTATCCTCTTCTGCTGTTCTGGAAAGAAACAG
ACCAGTGGTATGGAGTATAAGAAAACTGATGCACCTCAACCGGATGTGAAGGAAGAGGAAGAAGAGAAGGAAGAGGAAAAGGAC
AAGGGAGATGAGGAGGAGGAAGGAGAAGAGAAACTTGAAGAGAAACAGAAAAGTGATGCTGAAGAAGATGGTGGCACTGTCAGT
CAAGAGGAGGAAGACAGAAAACCTAAAGCAGAGGAGGATGAAATTTTGAACAGATCACCAAGAAACAGAAAGCCACGAAGAGAG
GTGAGCAAGGGCGAGGAGCTGTTCACCGGGGTGGTGCCCATCCTGGTCGAGCTGGACGGCGACGTaAACGGCCACAAGTTCAGC
GTGTCCGGCGAGGGCGAGGGCGATGCCACCTACGGCAAGCTGACCCTGAAGTTCATCTGCACCACCGGCAAGCTGCCCGTGCCC
TGGCCCACCCTCGTGACCACCCTGACCTACGGCGTGCAGTGCTTCAGCCGCTACCCCGACCACATGAAGCAGCACGACTTCTTC
AAGTCCGCCATGCCCGAAGGCTACGTCCAGGAGCGCACCATCTTCTTCAAGGACGACGGCAACTACAAGACCCGCGCCGAGGTG
AAGTTCGAGGGCGACACCCTGGTGAACCGCATCGAGCTGAAGGGCATCGACTTCAAGGAGGACGGCAACATCCTGGGGCACAAG
CTGGAGTACAACTACAACAGCCACAACGTCTATATCATGGCCGACAAGCAGAAGAACGGCATCAAGGTGAACTTCAAGATCCGC
CACAACATCGAGGACGGCAGCGTGCAGCTCGCCGACCACTACCAGCAGAACACCCCCATCGGCGACGGCCCCGTGCTGCTGCCC
GACAACCACTACCTGAGCACCCAGTCCGCCCTGAGCAAAGACCCCAACGAGAAGCGCGATCACATGGTCCTGCTGGAGTTCGTG
ACCGCCGCCGGGATCACTCtCGGCATGGACGAGCTGTACAAGtgaTTGTGTATGCGTTAATAAAAAGAAGGAACTCGTA
T44-TOP-uATG-TOM20-mCherry-GGGGS4-CatB-eGFP
(SEQ ID NO: 79)
GATCCGCCATCGTGGGTGAGTGTtagCTCTGTGGCCGCGCTCTGGCTAGTGGCGCTACGCGTCGCTCTCACGGGTGTCGTCGGA
TCTAATCCGTCTCTTTTCGAtagCAGGTGGAGCCGCCGCCACGAtggtgggacggaacagcgccatcgctgcaggagtgtgcgg
tgccacttcatagggtactgcatctactttgaccgcaaaaggaggagtgaccccaacCTCGAGGTGAGCAAGGGCGAGGAGGAT
AACATGGCCATCATCAAGGAGTTCATGCGCTTCAAGGTGCACATGGAGGGCTCCGTGAACGGCCACGAGTTCGAGATCGAGGGC
GAGGGCGAGGGCCGCCCCTACGAGGGCACCCAGACCGCCAAGCTGAAGGTGACCAAGGGTGGCCCCCTGCCCTTCGCCTGGGAC
ATCCTGTCCCCTCAGTTCATGTACGGCTCCAAGGCCTACGTGAAGCACCCCGCCGACATCCCCGACTACTTGAAGCTGTCCTTC
CCCGAGGGCTTCAAGTGGGAGCGCGTGATGAACTTCGAGGACGGCGGCGTGGTGACCGTGACCCAGGACTCCTCCCTGCAGGAC
GGCGAGTTCATCTACAAGGTGAAGCTGCGCGGCACCAACTTCCCCTCCGACGGCCCCGTAATGCAGAAGAAGACCATGGGCTGG
GAGGCCTCCTCCGAGCGGATGTACCCCGAGGACGGCGCCCTGAAGGGCGAGATCAAGCAGAGGCTGAAGCTGAAGGACGGCGGC
CACTACGACGCTGAGGTCAAGACCACCTACAAGGCCAAGAAGCCCGTGCAGCTGCCCGGCGCCTACAACGTCAACATCAAGTTG
GACATCACCTCCCACAACGAGGACTACACCATCGTGGAACAGTACGAACGCGCCGAGGGCCGCCACTCCACCGGCGGCATGGAC
GAGCTGTACAAGGGAGGTGGAGGCAGCGGAGGCGGGGGCAGTGGAGGAGGGGGTTCCGGTGGTGGTGGTAGTATGTGGTGGTCC
TTGATCCTTCTTTCTTGCCTGCTGGCACTGACCAGTGCCCATGACAAGCCTTCCTTCCACCCGCTGTCGGATGACCTGATTAAC
TATATCAACAAACAGAATACAACATGGCAGGCTGGACGCAACTTCTACAATGTTGACATAAGCTATCTGAAGAAGCTGTGTGGC
ACTGTCCTGGGTGGACCCAAACTGCCAGGAAGGGTTGCGTTCGGTGAGGACATAGATCTACCTGAAACCTTTGATGCACGGGAA
CAATGGTCCAACTGCCCGACCATTGGACAGATTAGAGACCAGGGCTCCTGCGGCTCTTGTTGGGCATTTGGGGCAGTGGAAGCC
ATTTCTGACCGAACCTGCATTCACACCAATGGCCGAGTCAACGTGGAGGTGTCTGCTGAAGACCTGCTTACTTGCTGTGGTATC
CAGTGTGGGGACGGCTGTAATGGTGGCTATCCCTCTGGAGCATGGAGCTTCTGGACAAAAAAAGGCCTGGTTTCAGGTGGAGTC
TACAATTCTCATGTAGGCTGCTTACCATACACCATCCCTCCCTGCGAGCACCATGTCAATGGCTCCCGTCCCCCATGCACTGGA
GAAGGAGATACTCCCAGGTGCAACAAGAGCTGTGAAGCTGGCTACTCCCCATCCTACAAAGAGGATAAGCACTTTGGGTACACT
TCCTACAGCGTGTCTAACAGTGTGAAGGAGATCATGGCAGAAATCTACAAAAATGGCCCAGTGGAGGGTGCCTTCACTGTGTTT
TCTGACTTCTTGACTTACAAATCAGGAGTATACAAGCATGAAGCCGGTGATATGATGGGTGGCCACGCCATCCGCATCCTGGGC
TGGGGAGTAGAGAATGGAGTTCCCTACTGGCTGGCAGCCAACTCTTGGAACCTTGACTGGGGTGATAATGGCTTCTTTAAAATC
CTCAGAGGAGAAAACCACTGTGGCATTGAATCAGAAATTGTGGCTGGAATCCCACGCACTGACCAGTACTGGGGAAGATTCGTG
AGCAAGGGCGAGGAGCTGTTCACCGGGGTGGTGCCCATCCTGGTCGAGCTGGACGGCGACGTaAACGGCCACAAGTTCAGCGTG
TCCGGCGAGGGCGAGGGCGATGCCACCTACGGCAAGCTGACCCTGAAGTTCATCTGCACCACCGGCAAGCTGCCCGTGCCCTGG
CCCACCCTCGTGACCACCCTGACCTACGGCGTGCAGTGCTTCAGCCGCTACCCCGACCACATGAAGCAGCACGACTTCTTCAAG
TCCGCCATGCCCGAAGGCTACGTCCAGGAGCGCACCATCTTCTTCAAGGACGACGGCAACTACAAGACCCGCGCCGAGGTGAAG
TTCGAGGGCGACACCCTGGTGAACCGCATCGAGCTGAAGGGCATCGACTTCAAGGAGGACGGCAACATCCTGGGGCACAAGCTG
GAGTACAACTACAACAGCCACAACGTCTATATCATGGCCGACAAGCAGAAGAACGGCATCAAGGTGAACTTCAAGATCCGCCAC
AACATCGAGGACGGCAGCGTGCAGCTCGCCGACCACTACCAGCAGAACACCCCCATCGGCGACGGCCCCGTGCTGCTGCCCGAC
AACCACTACCTGAGCACCCAGTCCGCCCTGAGCAAAGACCCCAACGAGAAGCGCGATCACATGGTCCTGCTGGAGTTCGTGACC
GCCGCCGGGATCACTCtCGGCATGGACGAGCTGTACAAGtgaTTGTGTATGCGTTAATAAAAAGAAGGAACTCGTA
T44-TOP-uATG-TOM20-mCherry-GGGGS4-NLS-eGFP-NLS
(SEQ ID NO: 80)
GATCCGCCATCGTGGGTGAGTGTtagCTCTGTGGCCGCGCTCTGGCTAGTGGCGCTACGCGTCGCTCTCACGGGTGTCGTCGGA
TCTAATCCGTCTCTTTTCGAtagCAGGTGGAGCCGCCGCCACGAtggtgggacggaacagcgccatcgctgcaggagtgtgcgg
tgccacttcatagggtactgcatctactttgaccgcaaaaggaggagtgaccccaacCTCGAGGTGAGCAAGGGCGAGGAGGAT
AACATGGCCATCATCAAGGAGTTCATGCGCTTCAAGGTGCACATGGAGGGCTCCGTGAACGGCCACGAGTTCGAGATCGAGGGC
GAGGGCGAGGGCCGCCCCTACGAGGGCACCCAGACCGCCAAGCTGAAGGTGACCAAGGGTGGCCCCCTGCCCTTCGCCTGGGAC
ATCCTGTCCCCTCAGTTCATGTACGGCTCCAAGGCCTACGTGAAGCACCCCGCCGACATCCCCGACTACTTGAAGCTGTCCTTC
CCCGAGGGCTTCAAGTGGGAGCGCGTGATGAACTTCGAGGACGGCGGCGTGGTGACCGTGACCCAGGACTCCTCCCTGCAGGAC
GGCGAGTTCATCTACAAGGTGAAGCTGCGCGGCACCAACTTCCCCTCCGACGGCCCCGTAATGCAGAAGAAGACCATGGGCTGG
GAGGCCTCCTCCGAGCGGATGTACCCCGAGGACGGCGCCCTGAAGGGCGAGATCAAGCAGAGGCTGAAGCTGAAGGACGGCGGC
CACTACGACGCTGAGGTCAAGACCACCTACAAGGCCAAGAAGCCCGTGCAGCTGCCCGGCGCCTACAACGTCAACATCAAGTTG
GACATCACCTCCCACAACGAGGACTACACCATCGTGGAACAGTACGAACGCGCCGAGGGCCGCCACTCCACCGGCGGCATGGAC
GAGCTGTACAAGGGAGGTGGAGGCAGCGGAGGCGGGGGCAGTGGAGGAGGGGGTTCCGGTGGTGGTGGTAGTATGGCCCCAAAG
AAGAAGCGGAAGGTCGGTATCCACGGAGTCCCAGCAGCCGTGAGCAAGGGCGAGGAGCTGTTCACCGGGGTGGTGCCCATCCTG
GTCGAGCTGGACGGCGACGTaAACGGCCACAAGTTCAGCGTGTCCGGCGAGGGCGAGGGCGATGCCACCTACGGCAAGCTGACC
CTGAAGTTCATCTGCACCACCGGCAAGCTGCCCGTGCCCTGGCCCACCCTCGTGACCACCCTGACCTACGGCGTGCAGTGCTTC
AGCCGCTACCCCGACCACATGAAGCAGCACGACTTCTTCAAGTCCGCCATGCCCGAAGGCTACGTCCAGGAGCGCACCATCTTC
TTCAAGGACGACGGCAACTACAAGACCCGCGCCGAGGTGAAGTTCGAGGGCGACACCCTGGTGAACCGCATCGAGCTGAAGGGC
ATCGACTTCAAGGAGGACGGCAACATCCTGGGGCACAAGCTGGAGTACAACTACAACAGCCACAACGTCTATATCATGGCCGAC
AAGCAGAAGAACGGCATCAAGGTGAACTTCAAGATCCGCCACAACATCGAGGACGGCAGCGTGCAGCTCGCCGACCACTACCAG
CAGAACACCCCCATCGGCGACGGCCCCGTGCTGCTGCCCGACAACCACTACCTGAGCACCCAGTCCGCCCTGAGCAAAGACCCC
AACGAGAAGCGCGATCACATGGTCCTGCTGGAGTTCGTGACCGCCGCCGGGATCACTCtCGGCATGGACGAGCTGTACAAGAAG
CGTCCTGCTGCTACTAAGAAAGCTGGTCAAGCTAAGAAAAAGAAATAAGCGGCCGCTTGTGTATGCGTTAATAAAAAGAAGGAA
CTCGTA
5UTR-24
90nt unnatural 5′ UTR with GGG, kozak sequence (GCCACC) and minimal secondary
structure
(SEQ ID NO: 81)
GGGGAGAAGAGGGAACAGGACACAAGAGAUAAACAUAAACAUAAACGACAAGAAACACAUACAAAAGAAACAGGACAGAAAACA
GCCACC
5UTR-25
70nt unnatural 5′ UTR with GG, kozak sequence (GCCACC), minimal secondary structure
and modified nucleotide composition.
(SEQ ID NO: 82)
GGAAACACAAUAACAUAAUCAUACUACACAACUAACACAUACAUCACAUACACAUCACAUAACAGCCACC
5UTR-26
70nt unnatural 5′ UTR with GG, kozak sequence (GCCACC), minimal secondary structure
and modified nucleotide composition.
(SEQ ID NO: 83)
GGCUACACACUCUCACUCUCAUCACUCACUACUCACUCUCUCAUCACUCUCACAUCACAUCACUGCCACC
5UTR-27
Unnatural 5′ UTR with the same length and nucleotide composition as 5UTR-25 without
the microRNA target sites in 5UTR-25.
(SEQ ID NO: 84)
GGCAAAAAUCAAAAUCAAUCAUCAUCACAACAUCAACAAUCAAUCAUCAACACAUCAUCAAGACACCACC
5UTR-28
Unnatural 5′ UTR with the same length and nucleotide composition as 5UTR-18 without
the microRNA target sites in 5UTR-18.
(SEQ ID NO: 85)
GGAAGAGAUCAAAAGCAACAAAUCAAACAGAGAAACAAUUAGAACAAGAAACAGAAGACAACAAGCCACC
5UTR-29
Unnatural 5′ UTR with the same length and nucleotide composition as 5UTR-26 without
the microRNA target sites in 5UTR-26.
(SEQ ID NO: 86)
GGCAUCACACUCUCACUCUCAUCUCAACACUCCUCCUCAUUCCAAUCUCUCACACAUCCCAUUAGCCACC
3UTR-4
Modified 3UTR-1 with a functional motif A (underlined) appended to 3′ end.
(SEQ ID NO: 87)
UUGUGUAUGCGUUAAUAAAAAGAAGGAACUCGUAAAAACUCAAUGUAUUUCUGAGGAAGCGUGGUGCAUAAUGCCACGCAGCGU
CUGCAUAACUUUUAUUAUUUCUUUUAUUAAUCAACAAA
motif A
(SEQ ID NO: 88)
AAAACUCAAUGUAUUUCUGAGGAAGCGUGGUGCAUAAUGCCACGCAGCGUCUGCAUAACUUUUAUUAUUUCUUUUAUUAAUCAA
CAAA
3UTR-5
Modified 3UTR-1 with a functional motif B (underlined) appended to 3′ end.
(SEQ ID NO: 89)
UUGUGUAUGCGUUAAUAAAAAGAAGGAACUCGUAUAUGUCUGUUUUUGUAUCUUUAUGCUGUAUUUUAACACUUUGUAUUACUU
AGGUUAUU
Motif B
(SEQ ID NO: 90)
UAUGUCUGUUUUUGUAUCUUUAUGCUGUAUUUUAACACUUUGUAUUACUUAGGUUAUU
3UTR-6
Modified 3UTR-1 with a functional motif C (underlined) appended to 3′ end.
(SEQ ID NO: 91)
UUGUGUAUGCGUUAAUAAAAAGAAGGAACUCGUAAACUCCAGGACUGUAUUUGUGACUAAUUGUAUAACAGGUU
Motif C
(SEQ ID NO: 92)
AACUCCAGGACUGUAUUUGUGACUAAUUGUAUAACAGGUU
COVID-19 mRNA vaccine 1
Full sequence of the mRNA utilizing 5UTR-27, 3UTR-4, and 120A tail to express the
coronavirus (COVID-19) spike protein as an antigen
(SEQ ID NO: 93)
GGCAAAAAUCAAAAUCAAUCAUCAUCACAACAUCAACAAUCAAUCAUCAACACAUCAUCAAGACACCACCAUGGGGGUUAAGGU
GCUCUUCGCGCUCAUCUGUAUUGCUGUGGCGGAAGCAGUUAAUCUUACAACCAGAACUCAAUUACCCCCUGCAUACACUAAUUC
UUUCACACGUGGUGUUUAUUACCCUGACAAAGUUUUCAGAUCCUCAGUUUUACAUUCAACUCAGGACUUGUUCUUACCUUUCUU
UUCCAAUGUUACUUGGUUCCAUGCUAUACAUGUCUCUGGGACCAAUGGUACUAAGAGGUUUGAUAACCCUGUCCUACCAUUUAA
UGAUGGUGUUUAUUUUGCUUCCACUGAGAAGUCUAACAUAAUAAGAGGCUGGAUUUUUGGUACUACUUUAGAUUCGAAGACCCA
GUCCCUACUUAUUGUUAAUAACGCUACUAAUGUUGUUAUUAAAGUCUGUGAAUUUCAAUUUUGUAAUGAUCCAUUUUUGGGUGU
UUAUUACCACAAAAACAACAAAAGUUGGAUGGAAAGUGAGUUCAGAGUUUAUUCUAGUGCGAAUAAUUGCACUUUUGAAUAUGU
CUCUCAGCCUUUUCUUAUGGACCUUGAAGGAAAACAGGGUAAUUUCAAAAAUCUUAGGGAAUUUGUGUUUAAGAAUAUUGAUGG
UUAUUUUAAAAUAUAUUCUAAGCACACGCCUAUUAAUUUAGUGCGUGAUCUCCCUCAGGGUUUUUCGGCUUUAGAACCAUUGGU
AGAUUUGCCAAUAGGUAUUAACAUCACUAGGUUUCAAACUUUACUUGCUUUACAUAGAAGUUAUUUGACUCCUGGUGAUUCUUC
UUCAGGUUGGACAGCUGGUGCUGCAGCUUAUUAUGUGGGUUAUCUUCAACCUAGGACUUUUCUAUUAAAAUAUAAUGAAAAUGG
AACCAUUACAGAUGCUGUAGACUGUGCACUUGACCCUCUCUCAGAAACAAAGUGUACGUUGAAAUCCUUCACUGUAGAAAAAGG
AAUCUAUCAAACUUCUAACUUUAGAGUCCAACCAACAGAAUCUAUUGUUAGAUUUCCUAAUAUUACAAACUUGUGCCCUUUUGG
UGAAGUUUUUAACGCCACCAGAUUUGCAUCUGUUUAUGCUUGGAACAGGAAGAGAAUCAGCAACUGUGUUGCUGAUUAUUCUGU
CCUAUAUAAUUCCGCAUCAUUUUCCACUUUUAAGUGUUAUGGAGUGUCUCCUACUAAAUUAAAUGAUCUCUGCUUUACUAAUGU
CUAUGCAGAUUCAUUUGUAAUUAGAGGUGAUGAAGUCAGACAAAUCGCUCCAGGGCAAACUGGAAAGAUUGCUGAUUAUAAUUA
UAAAUUACCAGAUGAUUUUACAGGCUGCGUUAUAGCUUGGAAUUCUAACAAUCUUGAUUCUAAGGUUGGUGGUAAUUAUAAUUA
CCUGUAUAGAUUGUUUAGGAAGUCUAAUCUCAAACCUUUUGAGAGAGAUAUUUCAACUGAAAUCUAUCAGGCCGGUAGCACACC
UUGUAAUGGUGUUGAAGGUUUUAAUUGUUACUUUCCUUUACAAUCAUAUGGUUUCCAACCCACUAAUGGUGUUGGUUACCAACC
AUACAGAGUAGUAGUACUUUCUUUUGAACUUCUACAUGCACCAGCAACUGUUUGUGGACCUAAAAAGUCUACUAAUUUGGUUAA
AAACAAAUGUGUCAAUUUCAACUUCAAUGGUUUAACAGGCACAGGUGUUCUUACUGAGUCUAACAAAAAGUUUCUGCCUUUCCA
ACAAUUUGGCAGAGACAUUGCUGACACUACUGAUGCUGUCCGUGAUCCACAGACACUUGAGAUUCUUGACAUUACACCAUGUUC
UUUUGGUGGUGUCAGUGUUAUAACACCAGGAACAAAUACUUCUAACCAGGUUGCUGUUCUUUAUCAGGAUGUUAACUGCACAGA
AGUCCCUGUUGCUAUUCAUGCAGAUCAACUUACUCCUACUUGGCGUGUUUAUUCUACAGGUUCUAAUGUUUUUCAAACACGUGC
AGGCUGUUUAAUAGGGGCUGAACAUGUCAACAACUCAUAUGAGUGUGACAUACCCAUUGGUGCAGGUAUAUGCGCUAGUUAUCA
GACUCAGACUAAUUCUCCUCGGCGGGCACGUAGUGUAGCUAGUCAAUCCAUCAUUGCCUACACUAUGUCACUUGGUGCAGAAAA
UUCAGUUGCUUACUCUAAUAACUCUAUUGCCAUACCCACAAAUUUUACUAUUAGUGUUACCACAGAAAUUCUACCAGUGUCUAU
GACCAAGACAUCAGUAGAUUGUACAAUGUACAUUUGUGGUGAUUCAACUGAAUGCAGCAAUCUUUUGUUGCAAUAUGGCAGUUU
UUGUACACAAUUAAACCGUGCUUUAACUGGAAUAGCUGUUGAACAAGACAAAAACACCCAAGAAGUUUUUGCACAAGUCAAACA
AAUUUACAAAACACCACCAAUUAAAGAUUUUGGUGGUUUUAAUUUUUCACAAAUAUUACCAGAUCCAUCAAAACCAAGCAAGAG
GUCAUUUAUUGAAGAUCUACUUUUCAACAAAGUGACACUUGCAGAUGCUGGCUUCAUCAAACAAUAUGGUGAUUGCCUUGGUGA
UAUUGCUGCUAGAGACCUCAUUUGUGCACAAAAGUUUAACGGCCUUACUGUUUUGCCACCUUUGCUCACAGAUGAAAUGAUUGC
UCAAUACACUUCUGCACUGUUAGCGGGUACAAUCACUUCUGGUUGGACCUUUGGUGCAGGUGCUGCAUUACAAAUACCAUUUGC
UAUGCAAAUGGCUUAUAGGUUUAAUGGUAUUGGAGUUACACAGAAUGUUCUCUAUGAGAACCAAAAAUUGAUUGCCAACCAAUU
UAAUAGUGCUAUUGGCAAAAUUCAAGACUCACUUUCUUCCACAGCAAGUGCACUUGGAAAACUUCAAGAUGUGGUCAACCAAAA
UGCACAAGCUUUAAACACGCUUGUUAAACAACUUAGCUCCAAUUUUGGUGCAAUUUCAAGUGUUUUAAAUGAUAUCCUUUCACG
UCUUGACAAAGUUGAGGCUGAAGUGCAAAUUGAUAGGUUGAUCACAGGCAGACUUCAAAGUUUGCAGACAUAUGUGACUCAACA
AUUAAUUAGAGCUGCAGAAAUCAGAGCUUCUGCUAAUCUUGCUGCUACUAAAAUGUCAGAGUGUGUACUUGGACAAUCAAAAAG
AGUUGAUUUUUGUGGAAAGGGCUAUCAUCUUAUGUCCUUCCCUCAGUCAGCACCUCAUGGUGUAGUCUUCUUGCAUGUGACUUA
UGUCCCUGCACAAGAAAAGAACUUCACAACUGCUCCUGCCAUUUGUCAUGAUGGAAAAGCACACUUUCCUCGUGAAGGUGUCUU
UGUUUCAAAUGGCACACACUGGUUUGUAACACAAAGGAAUUUUUAUGAACCACAAAUCAUUACUACAGACAACACAUUUGUGUC
UGGUAACUGUGAUGUUGUAAUAGGAAUUGUCAACAACACAGUUUAUGAUCCUUUGCAACCUGAAUUAGACUCAUUCAAGGAGGA
GUUAGAUAAAUAUUUUAAGAAUCAUACAUCACCAGAUGUUGAUUUAGGUGACAUCUCUGGCAUUAAUGCUUCAGUUGUAAACAU
UCAAAAAGAAAUUGACCGCCUCAAUGAGGUUGCCAAGAAUUUAAAUGAAUCUCUCAUCGAUCUCCAAGAACUUGGAAAGUAUGA
GCAGUAUAUAAAAUGGCCAUGGUACAUUUGGCUAGGUUUUAUAGCUGGCUUGAUUGCCAUAGUAAUGGUGACAAUUAUGCUUUG
CUGUAUGACCAGUUGCUGUAGUUGUCUCAAGGGCUGUUGUUCUUGUGGAUCCUGCUGCAAAUUUGAUGAAGACGACUCUGAGCC
AGUGCUCAAAGGAGUCAAAUUACAUUACACAGGCGGCGGAGGUUCUGAUUACAAGGACGAUGAUGAUAAAUAAUUGUGUAUGCG
UUAAUAAAAAGAAGGAACUCGUAAAAACUCAAUGUAUUUCUGAGGAAGCGUGGUGCAUAAUGCCACGCAGCGUCUGCAUAACUU
UUAUUAUUUCUUUUAUUAAUCAACAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAA
COVID-19 mRNA vaccine 2
Full sequence of the mRNA utilizing 5UTR-27, 3UTR-4, and 120A tail to express the
coronavirus (COVID-19) receptor binding domain (RBD) of the spike protein as an
antigen
(SEQ ID NO: 94)
GGCAAAAAUCAAAAUCAAUCAUCAUCACAACAUCAACAAUCAAUCAUCAACACAUCAUCAAGACACCACCAUGGGGGUUAAGGU
GCUCUUCGCGCUCAUCUGUAUUGCUGUGGCGGAAGCAAAUAUUACAAACUUGUGCCCUUUUGGUGAAGUUUUUAACGCCACCAG
AUUUGCAUCUGUUUAUGCUUGGAACAGGAAGAGAAUCAGCAACUGUGUUGCUGAUUAUUCUGUCCUAUAUAAUUCCGCAUCAUU
UUCCACUUUUAAGUGUUAUGGAGUGUCUCCUACUAAAUUAAAUGAUCUCUGCUUUACUAAUGUCUAUGCAGAUUCAUUUGUAAU
UAGAGGUGAUGAAGUCAGACAAAUCGCUCCAGGGCAAACUGGAAAGAUUGCUGAUUAUAAUUAUAAAUUACCAGAUGAUUUUAC
AGGCUGCGUUAUAGCUUGGAAUUCUAACAAUCUUGAUUCUAAGGUUGGUGGUAAUUAUAAUUACCUGUAUAGAUUGUUUAGGAA
GUCUAAUCUCAAACCUUUUGAGAGAGAUAUUUCAACUGAAAUCUAUCAGGCCGGUAGCACACCUUGUAAUGGUGUUGAAGGUUU
UAAUUGUUACUUUCCUUUACAAUCAUAUGGUUUCCAACCCACUAAUGGUGUUGGUUACCAACCAUACAGAGUAGUAGUACUUUC
UUUUGAACUUCUACAUGCACCAGCAACUGUUGGCGGCGGAGGUUCUGAUUACAAGGACGAUGAUGAUAAAUAAUUGUGUAUGCG
UUAAUAAAAAGAAGGAACUCGUAAAAACUCAAUGUAUUUCUGAGGAAGCGUGGUGCAUAAUGCCACGCAGCGUCUGCAUAACUU
UUAUUAUUUCUUUUAUUAAUCAACAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAA
COVID-19 mRNA vaccine 3
Full sequence of the mRNA utilizing 5UTR-27, 3UTR-4, and 120A tail to express the
coronavirus (COVID-19) envelope protein as an antigen
(SEQ ID NO: 95)
GGCAAAAAUCAAAAUCAAUCAUCAUCACAACAUCAACAAUCAAUCAUCAACACAUCAUCAAGACACCACCAUGGGGGUUAAGGU
GCUCUUCGCGCUCAUCUGUAUUGCUGUGGCGGAAGCAUACUCAUUCGUUUCGGAAGAGACAGGUACGUUAAUAGUUAAUAGCGU
ACUUCUUUUUCUUGCUUUCGUGGUAUUCUUGCUAGUUACACUAGCCAUCCUUACUGCGCUUCGAUUGUGUGCGUACUGCUGCAA
UAUUGUUAACGUGAGUCUUGUAAAACCUUCUUUUUACGUUUACUCUCGUGUUAAAAAUCUGAAUUCUUCUAGAGUUCCUGAUCU
UCUGGUCGGCGGAGGAGGGUCAUACACCGACAUAGAGAUGAAUCGGCUUGGCAAAUAAUUGUGUAUGCGUUAAUAAAAAGAAGG
AACUCGUAAAAACUCAAUGUAUUUCUGAGGAAGCGUGGUGCAUAAUGCCACGCAGCGUCUGCAUAACUUUUAUUAUUUCUUUUA
UUAAUCAACAAAAAAAAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAA
COVID-19 mRNA vaccine 4
Full sequence of the mRNA utilizing 5UTR-27, 3UTR-4, and 120A tail to express the
coronavirus (COVID-19) membrane protein as an antigen
(SEQ ID NO: 96)
GGCAAAAAUCAAAAUCAAUCAUCAUCACAACAUCAACAAUCAAUCAUCAACACAUCAUCAAGACACCACCAUGGGGGUUAAGGU
GCUCUUCGCGCUCAUCUGUAUUGCUGUGGCGGAAGCAGCAGAUUCCAACGGUACUAUUACCGUUGAAGAGCUUAAAAAGCUCCU
UGAACAAUGGAACCUAGUAAUAGGUUUCCUAUUCCUUACAUGGAUUUGUCUUCUACAAUUUGCCUAUGCCAACAGGAAUAGGUU
UUUGUAUAUAAUUAAGUUAAUUUUCCUCUGGCUGUUAUGGCCAGUAACUUUAGCUUGUUUUGUGCUUGCUGCUGUUUACAGAAU
AAAUUGGAUCACCGGUGGAAUUGCUAUCGCAAUGGCUUGUCUUGUAGGCUUGAUGUGGCUCAGCUACUUCAUUGCUUCUUUCAG
ACUGUUUGCGCGUACGCGUUCCAUGUGGUCAUUCAAUCCAGAAACUAACAUUCUUCUCAACGUGCCACUCCAUGGCACUAUUCU
GACCAGACCGCUUCUAGAAAGUGAACUCGUAAUCGGAGCUGUGAUCCUUCGUGGACAUCUUCGUAUUGCUGGACACCAUCUAGG
ACGCUGUGACAUCAAGGACCUGCCUAAAGAAAUCACUGUUGCUACAUCACGAACGCUUUCUUAUUACAAAUUGGGAGCUUCGCA
GCGUGUAGCAGGUGACUCAGGUUUUGCUGCAUACAGUCGCUACAGGAUUGGCAACUAUAAAUUAAACACAGACCAUUCCAGUAG
CAGUGACAAUAUUGCUUUGCUUGUACAGGGCGGAGGAGGGUCAUACACCGACAUAGAGAUGAAUCGGCUUGGCAAAUAAUUGUG
UAUGCGUUAAUAAAAAGAAGGAACUCGUAAAAACUCAAUGUAUUUCUGAGGAAGCGUGGUGCAUAAUGCCACGCAGCGUCUGCA
UAACUUUUAUUAUUUCUUUUAUUAAUCAACAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAAAAAAA
COVID-19 mRNA vaccine 5
Full sequence of the mRNA utilizing 5UTR-27, 3UTR-4, and 120A tail to express the
coronavirus (COVID-19) nucleocapsid protein as an antigen
(SEQ ID NO: 97)
GGCAAAAAUCAAAAUCAAUCAUCAUCACAACAUCAACAAUCAAUCAUCAACACAUCAUCAAGACACCACCAUGGGGGUUAAGGU
GCUCUUCGCGCUCAUCUGUAUUGCUGUGGCGGAAGCAUCUGAUAAUGGACCCCAAAAUCAGCGAAAUGCACCCCGCAUUACGUU
UGGUGGACCCUCAGAUUCAACUGGCAGUAACCAGAAUGGAGAACGCAGUGGGGCGCGAUCAAAACAACGUCGGCCCCAAGGUUU
ACCCAAUAAUACUGCGUCUUGGUUCACCGCUCUCACUCAACAUGGCAAGGAAGACCUUAAAUUCCCUCGAGGACAAGGCGUUCC
AAUUAACACCAAUAGCAGUCCAGAUGACCAAAUUGGCUACUACCGAAGAGCUACCAGACGAAUUCGUGGUGGUGACGGUAAAAU
GAAAGAUCUCAGUCCAAGAUGGUAUUUCUACUACCUAGGAACUGGGCCAGAAGCUGGACUUCCCUAUGGUGCUAACAAAGACGG
CAUCAUAUGGGUUGCAACUGAGGGAGCCUUGAAUACACCAAAAGAUCACAUUGGCACCCGCAAUCCUGCUAACAAUGCUGCAAU
CGUGCUACAACUUCCUCAAGGAACAACAUUGCCAAAAGGCUUCUACGCAGAAGGGAGCAGAGGCGGCAGUCAAGCCUCUUCUCG
UUCCUCAUCACGUAGUCGCAACAGUUCAAGAAAUUCAACUCCAGGCAGCAGUAGGGGAACUUCUCCUGCUAGAAUGGCUGGCAA
UGGCGGUGAUGCUGCUCUUGCUUUGCUGCUGCUUGACAGAUUGAACCAGCUUGAGAGCAAAAUGUCUGGUAAAGGCCAACAACA
ACAAGGCCAAACUGUCACUAAGAAAUCUGCUGCUGAGGCUUCUAAGAAGCCUCGGCAAAAACGUACUGCCACUAAAGCAUACAA
UGUAACACAAGCUUUCGGCAGACGUGGUCCAGAACAAACCCAAGGAAAUUUUGGGGACCAGGAACUAAUCAGACAAGGAACUGA
UUACAAACAUUGGCCGCAAAUUGCACAAUUUGCCCCCAGCGCUUCAGCGUUCUUCGGAAUGUCGCGCAUUGGCAUGGAAGUCAC
ACCUUCGGGAACGUGGUUGACCUACACAGGUGCCAUCAAAUUGGAUGACAAAGAUCCAAAUUUCAAAGAUCAAGUCAUUUUGCU
GAAUAAGCAUAUUGACGCAUACAAAACAUUCCCACCAACAGAGCCUAAAAAGGACAAAAAGAAGAAGGCUGAUGAAACUCAAGC
CUUACCGCAGAGACAGAAGAAACAGCAAACUGUGACUCUUCUUCCUGCUGCAGAUUUGGAUGAUUUCUCCAAACAAUUGCAACA
AUCCAUGAGCAGUGCUGACUCAACUCAGGCCGGCGGAGGAGGGUCAUACACCGACAUAGAGAUGAAUCGGCUUGGCAAAUAAUU
GUGUAUGCGUUAAUAAAAAGAAGGAACUCGUAAAAACUCAAUGUAUUUCUGAGGAAGCGUGGUGCAUAAUGCCACGCAGCGUCU
GCAUAACUUUUAUUAUUUCUUUUAUUAAUCAACAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAAAAAAAAAA

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. An engineered mRNA comprising:
a first nucleic acid sequence comprising an RPS27A 5′ untranslated region (5′UTR) sequence or an engineered 5′ untranslated region (5′UTR) sequence;
a second nucleic acid sequence comprising a heterologous nucleic acid sequence; and
a third nucleic acid sequence comprising an RPS27A 3′ untranslated region (3′UTR) sequence.

2. The engineered mRNA of claim 1, wherein the RPS27A 5′UTR sequence or the engineered 5′UTR sequence is selected from the group comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 81, SEQ ID NO: 82, SEQ ID NO: 83, SEQ ID NO: 84, SEQ ID NO: 85, or SEQ ID NO: 86.

3. The engineered mRNA of claim 1 or 2, wherein the heterologous nucleic acid sequence encodes a target protein.

4. The engineered mRNA of claim 3, wherein the target protein comprises a fluorescent protein.

5. The engineered mRNA of claim 4, wherein the fluorescent protein comprises GFP or mCherry.

6. The engineered mRNA of claim 3, wherein the target protein comprises a viral protein.

7. The engineered mRNA of claim 6, wherein the viral protein is a COVID-19 protein.

8. The engineered mRNA of claim 3, wherein the target protein comprises a co-stimulatory molecule.

9. The engineered mRNA of claim 8, wherein the co-stimulatory molecule is selected from ICOS, CD28, CD27, HVEM, LIGHT, CD40L, 4-1BB, OX40, DR3, GITR, CD30, SLAM, CD2, CD226, Galectin9, TIM1, LFA1, B7-H2, B7-1, B7-2, CD70, LIGHT, HVEM, CD40, 4-1BBL, OX40L, TL1A, GITRL, CD30L, SLAM, CD48, CD58, CD155, CD112, CD80, CD86, ICOSL, TIM3, TIM4, ICAM1, or LFA3.

10. The engineered mRNA of any one of claims 1 to 9, wherein the RPS27A 3′UTR sequence is selected from the group comprising SEQ ID NO: 24, SEQ ID NO: 25, SEQ ID NO: 26, SEQ ID NO: 87, SEQ ID NO: 89, or SEQ ID NO: 91.

11. The engineered mRNA of any one of claims 1 to 10, comprising an RNA sequence selected from the group comprising SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, or SEQ ID NO: 40.

12. The engineered mRNA of any one of claims 1 to 10, comprising an RNA sequence selected from the group comprising SEQ ID NO: 93, SEQ ID NO: 94, SEQ ID NO: 95, SEQ ID NO: 96, or SEQ ID NO: 97.

13. The engineered mRNA of any one of claims 1 to 12, wherein the engineered mRNA comprises at least one chemically modified nucleotide.

14. The engineered mRNA of claim 13, wherein the at least one chemically modified nucleotide is a chemically modified nucleobase.

15. The engineered mRNA of claim 14, wherein the chemically modified nucleobase is pseudouridine.

16. A vector comprising a nucleic acid encoding the engineered mRNA of any one of claims 1 to 15.

17. A cell comprising the vector of claim 16.

18. A method of increasing protein expression, comprising the steps:
introducing into a cell an engineered mRNA, comprising:
a first nucleic acid sequence comprising an RPS27A 5′UTR sequence or an engineered 5′ untranslated region (5′UTR) sequence;
a second nucleic acid sequence comprising a heterologous nucleic acid sequence; and
a third nucleic acid sequence comprising an RPS27A 3′UTR sequence.

19. The method of claim 18, wherein the RPS27A 5′UTR sequence or the engineered 5′UTR sequence is selected from the group comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 81, SEQ ID NO: 82, SEQ ID NO: 83, SEQ ID NO: 84, SEQ ID NO: 85, or SEQ ID NO: 86.

20. The method of claim 18 or 19, wherein the heterologous nucleic acid sequence encodes a target protein.

21. The method of claim 20, wherein the target protein comprises a fluorescent protein.

22. The method of claim 21, wherein the fluorescent protein comprises GFP or mCherry.

23. The method of claim 20, wherein the target protein comprises a viral protein.

24. The method of claim 23, wherein the viral protein is a COVID-19 protein.

25. The method of claim 20, wherein the target protein comprises a co-stimulatory molecule.

26. The method of claim 25, wherein the co-stimulatory molecule is selected from ICOS, CD28, CD27, HVEM, LIGHT, CD40L, 4-1BB, OX40, DR3, GITR, CD30, SLAM, CD2, CD226, Galectin9, TIM1, LFA1, B7-H2, B7-1, B7-2, CD70, LIGHT, HVEM, CD40, 4-1BBL, OX40L, TL1A, GITRL, CD30L, SLAM, CD48, CD58, CD155, CD112, CD80, CD86, ICOSL, TIM3, TIM4, ICAM1, or LFA3.

27. The method of any one of claims 18 to 26, wherein the RPS27A 3′UTR sequence is selected from the group comprising SEQ ID NO: 24, SEQ ID NO: 25, SEQ ID NO: 26, SEQ ID NO: 87, SEQ ID NO: 89, or SEQ ID NO: 91.

28. The method of any one of claims 18 to 27, wherein the engineered mRNA comprises an RNA sequence selected from the group comprising SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, or SEQ ID NO: 40.

29. The method of any one of claims 18 to 27, wherein the engineered mRNA comprises an RNA sequence selected from the group comprising SEQ ID NO: 93, SEQ ID NO: 94, SEQ ID NO: 95, SEQ ID NO: 96, or SEQ ID NO: 97.

30. The method of any one of claims 18 to 29, wherein the engineered mRNA comprises at least one chemically modified nucleotide.

31. The method of claim 30, wherein the at least one chemically modified nucleotide is a chemically modified nucleobase.

32. The method of claim 31, wherein the chemically modified nucleobase is pseudouridine.
